(19) United States
(12) Patent Application Publication
(10) Pub. No.: US 2025/0295796 A1
(43) Pub. Date: Sep. 25, 2025

(54) CELL PENETRATING POLYPEPTIDES (CPPS) AND THEIR USE IN HUMAN THERAPY

(71) Applicants: RDP PHARMA AG, Romanshorn (CH); PAGS CO., LTD., Seoul (KR)

(72) Inventor: Christian KÜHNE, Tulin (AT)

(21) Appl. No.: 18/720,267

(22) PCT Filed: Dec. 15, 2022
(86) PCT No.: PCT/EP2022/086215

§ 371 Date: Jun. 14, 2024

(30) Foreign Application Priority Data
Dec. 16, 2021 (EP) 21215322.5
Jan. 11, 2022 (EP) 22150880.7

(51) Int. Cl. A61K 47/64; A61K 35/04; C07K 7/08

(52) U.S. Cl. CPC A61K 47/645; A61K 35/04; C07K 7/08

(57) ABSTRACT

The invention discloses a compound comprising or consisting of a polypeptide with the general formula (I): X₀GX₁X₂GX₃X₄X₅GX₆X₇X₈GX₉X₁₀X₁₁X₁₂X₁₃X₁₄, wherein G is glycine; X₀ is present or not and, if present, is an amino acid linker; X₁ is N or S, wherein N is asparagine and S is serine; X₂ is S or T, wherein S is serine and T is threonine; X₃ is present or not and, if present, is S, wherein S is serine; X₄ is present or not and, if present, is G, wherein G is glycine; X₅ is a basic polypeptide stretch consisting of 5 basic amino acid residues selected from R and K, wherein R is arginine and K is lysine, preferably wherein X₅ comprises at least 3 K amino acid residues, especially wherein X₅ is KKKKK or KRKKK; X₆ is a basic amino acid residue selected from R and K, wherein R is arginine and K is lysine, preferably wherein X₆ is K; X₇ is S or L, wherein S is serine and L is leucine; X₈ is present or not and, if present, is a polypeptide stretch consisting of the amino acid sequence GLGS, wherein G is glycine, L is leucine and S is serine; X₉ is a basic polypeptide stretch consisting of 3 basic amino acid residues selected from R and K, wherein R is arginine and K is lysine, preferably wherein X₉ comprises at least 2 K amino acid residues, especially wherein X₉ is KKK or KKR; X₁₀ is present or not and, if present, is L or a polypeptide stretch consisting of the amino acid sequence DPL or DPC, wherein L is leucine, D is aspartic acid, P is proline, and C is cysteine; X₁₁ is present or not and, if present, is a polypeptide stretch consisting of the amino acid sequence LR or GSGL, wherein L is leucine, R is arginine, G is glycine, and S is serine; X₁₂ is present or not and, if present, is a basic polypeptide stretch with at least 20% basic amino acid residues selected from K and R and at least a P residue, preferably wherein X₁₂ is selected from KYKPKL, KYKPKLGT, or GX₃X₄X₅GX₆X₇X₈GX₉X₁₀, wherein K, R, P, L, G, T, X₃, X₄, X₅, GX₆, X₇, X₈, GX₉, and X₁₀, are CA defined as above and wherein Y is tyrosine; X₁₃ is present or not and, if present, is a polypeptide stretch consisting of the amino acid sequences GS, GST, GST, GSG, GSTG, or GSGL, wherein G, S, T and L are defined as above; X₁₄ is present or not and, if present, is an amino acid linker; wherein the polypeptide has a length from 20 to 75 amino acid residues, preferably from 24 to 65 amino acid residues, especially from 25 to 60 amino acid residues.

The present invention relates to the field of cell penetrating polypeptides (CPPs) and their use in human therapy, especially in cancer therapy.

The use of small peptides that cross cellular membranes called CPPs or protein transduction domains (PTDs), enables cellular drug delivery of large molecules and can enhance drug delivery of small molecule compounds. Whereas in therapy small molecule compounds can cross cellular membranes by diffusion to a certain extent, the therapeutic use of macromolecules is limited by their poor penetration in tissues and their inability to cross the cellular membrane without appropriate drug delivery systems. Thus, the CPPs directed transport of macromolecules across biological membranes allows to develop a therapeutic drug delivery vehicle for miscellaneous applications. Multiple clinical trials are testing first generation CPP-mediated delivery of macromolecular CPP conjugates in a variety of indications. CPP carrier peptides are a class of short peptide sequences also known as protein transduction domains (PTDs), cell permeable polypeptides (CPPs) or membrane translocating sequences (MTSs). Their ability to ferry much larger molecules into cells makes them ideal tools for transferring polypeptides and other molecules into living cells for both research purposes and therapeutic applications. For example, CPPs have been reported to deliver therapeutic polypeptides, antisense oligonucleotides, liposomes, plasmids, nanoparticles, phages, and viruses into mammalian cells. CPPs are structurally highly diverse (many of the CPPs are highly cationic and arginine or lysine rich but besides that show little sequence homology with each other) but are known to the person skilled in the art as functional class of polypeptides. There are a number of examples known (as summarised recently e.g. in WO 2020/214846 A1, especially in table 1). Two specific cell penetrating peptides, GKRKKKGKLGKKRDP and GKRKKKGKGLGKKRDPCLRKYK are disclosed in de Coupade et al. (Biochem. J. 390 (2005): 407-418), WO 01/64738 A2 and Lee et al. (BBRC 419 (2012): 597-604). Both peptides are derived from the heparin-binding domain of epidermal growth factor-like growth factor. WO 2004/092194 A2 teaches the fusion of a single chain PvuII restriction endonuclease to a cell-penetrating peptide.

Many currently known CPPs have major limitation, such as poor tissue selectivity with concomitant off-target effects, toxicology constrains, medium potency demanding high therapeutic concentrations and broad tissue bio-distributions at effective dose. Moreover, these molecules are often highly vulnerable to proteases and thus exhibit stability problems under production and storage conditions, a drawback for an implementation in the context of a pharmaceutical drug product, limiting druggability of respective CPP conjugates. Consequently, these CPPs exhibit unfavourable pharmacokinetics parameters as carrier for a given payload drug substance. Putative therapeutic active concentrations are comparatively high in the medium to low μM range. Given the valuable principle for an active intracellular drug delivery comprised by CPP techniques, however due to said constrains rather poor druggability, an amelioration of these parameters seems profitable. Despite the promising results obtained with some CPP linked polymer therapeutics, stability problems and/or delivery problems compromise the efficacy of many such therapeutics. Polymer-linked payloads frequently become stacked in the late endosome and release into the compartment of interest shows low efficiently, demands high concentrations and does not allow to achieve a therapeutic effect without significant general toxicities.

Accordingly, there is a need for the development of improved CPPs where these limitations are improved. The prior art still reflects the need for novel, alternative CPPs, especially polymercontaining CPP-ligand conjugates and polymer-CPP compounds, which have the ability to efficiently translocate a biological membrane and provide cell-type selective and/or intracellular compartment selective delivery of molecules, and thus have utility in therapy and/or as research tools. Moreover, there is a demand in the art for CPP-linked polymer therapeutics having significantly lower toxicity, yet useful therapeutic efficiency, or delivery systems providing cell-type selective and/or intracellular compartment selective delivery of a broad range of therapeutic molecules, including biological macromolecules, small molecule drugs, or combinations of biological macromolecules and small molecule drugs.

It is an object of the present invention to provide improved CPPs with improved transport efficiency over the cellular membrane to enable successful delivery of payload molecules to an intracellular environment. It is a further object to provide efficient compounds comprising such CPPs which are suitable to treat medical disorders and diseases, preferably to treat cancer patients of various kinds, especially to treat cancer patients who already have metastases. More specifically, it is another object to provide CPP-delivered therapeutics having significantly lower toxicity, yet useful therapeutic efficiency, or delivery systems providing cell-type selective and/or intracellular compartment selective delivery of a broad range of therapeutic molecules, including biological macromolecules, small molecule drugs, or combinations of biological macromolecules and small molecule drugs. Another object of the present invention is to provide CPPs which have the ability to efficiently translocate a biological membrane and provide cell-type selective and/or intracellular compartment selective delivery of molecules, and thus have utility in therapy and/or as research tools.

Therefore, the present invention provides a compound comprising or consisting of a polypeptide with the general formula (I): X₀GX₁X₂GX₃X₄X₅GX₆X₇X₈GX₉X₁₀X₁₁X₁₂X₁₃X₁₄,
wherein
G is glycine;
X₀ is present or not and, if present, is an amino acid linker;
X₁ is N or S, wherein N is asparagine and S is serine;
X₂ is S or T, wherein S is serine and T is threonine;
X₃ is present or not and, if present, is S, wherein S is serine;
X₄ is present or not and, if present, is G, wherein G is glycine;
X₅ is a basic polypeptide stretch consisting of 5 basic amino acid residues selected from R and K, wherein R is arginine and K is lysine, preferably wherein X₅ comprises at least 3 K amino acid residues, especially wherein X₅ is KKKKK or KRKKK;
X₆ is a basic amino acid residue selected from R and K, wherein R is arginine and K is lysine, preferably wherein X₆ is K;
X₇ is S or L, wherein S is serine and L is leucine;
X₈ is present or not and, if present, is a polypeptide stretch consisting of the amino acid sequence GLGS, wherein G is glycine, L is leucine and S is serine;
X₉ is a basic polypeptide stretch consisting of 3 basic amino acid residues selected from R and K, wherein R is arginine and K is lysine, preferably wherein X₉ comprises at least 2 K amino acid residues, especially wherein X₉ is KKK or KKR;
X₁₀ is present or not and, if present, is L or a polypeptide stretch consisting of the amino acid sequence DPL or DPC, wherein L is leucine, D is aspartic acid, P is proline, and C is cysteine;
X₁₁ is present or not and, if present, is a polypeptide stretch consisting of the amino acid sequence LR or GSGL, wherein L is leucine, R is arginine, G is glycine, and S is serine;
X₁₂ is present or not and, if present, is a basic polypeptide stretch with at least 20% basic amino acid residues selected from K and R and at least a P residue, preferably wherein X₁₂ is selected from KYKPKL, KYKPKLGT, or GX₃X₄X₅GX₆X₇X₈GX₉X₁₀, wherein K, R, P, L, G, T, X₃, X₄, X₅, GX₆, X₇, X₈, GX₉, and X₁₀, are defined as above and wherein Y is tyrosine;
X₁₃ is present or not and, if present, is a polypeptide stretch consisting of the amino acid sequences GS, GST, GST, GSG, GSTG, or GSGL, wherein G, S, T and L are defined as above;
X₁₄ is present or not and, if present, is an amino acid linker; wherein the polypeptide has a length from 20 to 75 amino acid residues, preferably from 24 to 65 amino acid residues, especially from 25 to 60 amino acid residues.

The compound of the present invention comprises or consists of a new class of CPPs which have an unexpected cell penetrating and transmembrane transport properties which allow efficient transport of payload molecules (often also referred to as “target”, “targeting molecules”, “effective molecule”, “active molecule”, “active drug”, “active biologic”, etc.). The CPPs according to the present invention have also improved properties for addressing tumour cells, especially when they are covalently linked to a molecule effective in killing tumour cells. The suitability to use the CPPs according to the present invention for treating tumor patients resides in the efficient cell-penetrating function of the polypeptides according to the present invention.

The compounds provided with the present invention with the improved CPP moieties exhibit better efficiency during the membrane crossing and intracellular sorting processes. The CPPs according to the present invention are specifically suited as a platform to transport different payloads for different pharmaceutical applications. Moreover, the efficient CPPs according to the present invention also exhibit improved solubility in recombinant expression systems and show robust stability in production processes. This subsequently allows transfer and up-scale into API manufacturing processes of CPP-conjugates as compounds according to the present invention, bringing about pharmaceutical acceptable stability of a respective drug product. The advantages in the production process for the compounds according to the present invention are specifically helpful if the payload is a polypeptide. Most studies wherein PTD- or CPP-payload fusion polypeptides were investigated in bacteria resulted in problems such as polypeptide solubility, the formation of inclusion bodies and the lack of eukaryotic posttranslational modifications important for selective applications. These problems are not observed with the CPPs according to the present invention or at least significantly reduced with known CPPs. These differences in performance and efficiency of the CPPs according to the present invention also apply to the peptides GKRKKKGKLGKKRDP and GKRKKKGKGLGKKRDPCLRKYK.

The present invention also allows modifying payloads within the context of a selected CPP for better pharmaco-kinetic (PK) and pharmaco-dynamic (PD) properties. The present compounds are efficient in solving one or more (or all) the objects posed above.

The CPP conjugates according to the present invention enable a longer in vivo half-life; reduced immunogenicity, toxicity, and selective clearance rate; successful transportation across a cell membrane; protection against proteolysis; modification of electroosmotic flow; increased pH and thermal stability; a low volume of distribution and sustained adsorption from the injection site; and improved formulation properties of the polypeptide. These superior properties can increase effective potency, improve response to the drug, increase patient tolerance and reduce side effects, and reduce overall dosage. For example, many biological macromolecules, including proteins, peptides, polynucleotides and nucleic acids, have proven useful for the treatment of various health problems.

According to a preferred embodiment, the CPP moiety in the compound according to the present invention is a compound with the general formula (I), wherein X₁ is N, X₂ is S, X₃ is S, X₄ is G, X₅ is KKKKK or KRKKK, X₆ is K, X₉ is KKK or KKR, and X₁₀ is L or a polypeptide stretch consisting of the amino acid sequence DPL or DPC, wherein N, S, G, R, L, D, P and C are defined as above.

According to a preferred embodiment, the number of arginine or cysteine residues in the CPP moiety with the general formula (I). Preferably, the compound according to the present invention has a general formula (I) which contains a single arginine and/or a single cysteine or not more than two arginine residues and/or not more than two cysteine residues. According to a specifically preferred embodiment, the CPP has a general formula (I) which lacks an arginine and/or cysteine residue, especially wherein the general formula (I) lacks both, arginine and cysteine residues.

The replacement of arginine residues and/or the exchange of arginine residues with lysine residues has shown to improve the stability of the compounds according to the present invention, both by preventing protease degradation in vivo (i.e. when the compounds according to the present invention are applied to human patients) and by preventing degradation in the course of the production process. Exchange of arginine by another amino acid, especially by lysine may e.g. prevent a protease site.

This lack of cysteine residues in the CPP moiety is, of course, independent of the use of cysteine residues in linking moieties which links the CPP to a payload molecule (see below).

Moreover, cysteine residues may also be used as preferred points of PEGylation to provide PEGylated compounds according to the present invention.

According to another preferred embodiment, the compound according to the present invention comprises at least two polypeptides with the general formula (I), preferably wherein the CPP consists of two polypeptides with the general formula (I).

A specifically preferred embodiment of the present invention is a compound wherein the CPP is selected from the group

GNSGSGKKKKKGKGSGLGSGKKKDPL
(DDM34)
and
GNSGSGKKKKKGKGSGLGSGKKKDPLGSGLGSGK
KKKKGKGSGLGSGKKKDPLGST
(DDM36).

Preferred specific examples of the CPPs according to the present invention are

GNSGSGKRKKKGKGLGKKRDPCLRKYKPKLGTGSTG
(HHHHHH)
(DDM11),
GNSGKRKKKGKGSGLGSGKKRDPCLRKYKPKLGSTG
(HHHHHH)
(DDM18),
GNSGSGKRKKKGKGLGKKRDPCLRKYKPKLGTGSTG
(HHHHHH)
(DDM21),
GNSGSGKRKKKGKGSGLGSGKKRDPLGSTG
(HHHHHH)
(DDM26),
GNSG(HHHHHH)GSTGGKRKKKGKGSGLGSG
KKRDPL
(DDM27),
GNSGSGKRKKKGKGSGLGSGKKKDPLGSTG
(HHHHHH)
(DDM28),
GNSG(HHHHHH)GSTGGKRKKKGKGSGLGS
GKKKDPL
(DDM29),
GNSGSGKKKKKGKGSGLGSGKKRDPLGSTG
(HHHHHH)
(DDM30),
GNSG(HHHHHH)GSTGGKKKKKGKGSGLGS
GKKRDPL
(DDM31),
GNSGSGKKKKKGKGSGLGSGKKKDPLGSTG
(HHHHHH)
(DDM32),
GNSGHHHHHH)GSTGGKKKKKGKGSGLGSG
(KKKDPL
(DDM33),
GNSGSGKKKKKGKGSGLGSGKKKDPL
(DDM34),
GNSGSGKKKKKGKGSGLGSGKKKLGSTG
(HHHHHH)
(DDM35),
GNSGSGKKKKKGKGSGLGSGKKKDPLGSGLGS
GKKKKKGKGSGLGSGKKKDPLGST
(DDM36),
and
GNSGSGKKKKKGKGSGLGSGKKKDPLGSGLGSGK
KKKKGKGSGLGSGKKKDPLGSTG(HHHHHH)GST
(DDM44),

wherein (HHHHHH; a histidine tag) may be present or not; preferably

GNSGSGKRKKKGKGLGKKRDPCLRKYKPKLGTGSTG
(DDM11),
GNSGKRKKKGKGSGLGSGKKRDPCLRKYKPKLGSTG
(DDM18),
GNSGSGKRKKKGKGLGKKRDPCLRKYKPKLGTGSTG
(DDM21),
GNSGSGKRKKKGKGSGLGSGKKRDPLGSTG
(DDM26),
GNSGGSTGGKRKKKGKGSGLGSGKKRDPL
(DDM27),
GNSGSGKRKKKGKGSGLGSGKKKDPLGSTG
(DDM28),
GNSGGSTGGKRKKKGKGSGLGSGKKKDPL
(DDM29),
GNSGSGKKKKKGKGSGLGSGKKRDPLGSTG
(DDM30),
GNSGGSTGGKKKKKGKGSGLGSGKKRDPL
(DDM31),
GNSGSGKKKKKGKGSGLGSGKKKDPLGSTG
(DDM32),
GNSGSGKKKKKGKGSGLGSGKKKDPL
(DDM34),
GNSGSGKKKKKGKGSGLGSGKKKDPLGSGLGSGKKK
KKGKGSGLGSGKKKDPLGST
(DDM36),
and
GNSGSGKKKKKGKGSGLGSGKKKDPLGSGLGSGKKK
KKGKGSGLGSGKKKDPLGSTGGST
(DDM44), especially
GNSGSGKKKKKGKGSGLGSGKKKDPLGSGLGSGKKK
KKGKGSGLGSGKKKDPLGST
(DDM36),
and
GNSGSGKKKKKGKGSGLGSGKKKDPLGSGLGS
GKKKKKGKGSGLGSGKKKDPLGSTGGST
(DDM44)

According to a preferred embodiment, the polypeptide with the general formula (I) in the compound according to the present invention is covalently coupled to the C-terminus of a payload molecule to be delivered into a biological cell, preferably a compound selected from the group

(CROMOC01)
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKI-
YGNSGSGKKKKKGKGSGLGSGKKKDPLGSGLGSGKKKKKGKGSGLGSGKKKDPLGST,
(CROMOC02)
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKI-
YGNSGSGKKKKKGKGSGLGSGKKKDPLGSGLGSGKKKKKGKGSGLGSGKKKDPLGST,
(CROMOC36)
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
(CROMOC60)
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
(CROMOC62)
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
(CROMOC63)
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGCCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
(CROMOC70)
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
(CROMOC87)
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
(CROMOC88)
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
and
(CROMOC89)
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST.

The term “coupled” or “covalently coupled” preferably relates to a covalent peptidic coupling, e.g. the coupling of two moieties of the compound according to the present invention via peptidic linkage thereby forming a polypeptide. Of course, other coupling (e.g. via S—S bonding or other chemical (covalent) coupling) may also be used to link the different moieties in the present compound; however, peptidic bonds are the preferred way to (covalently) couple the moieties of the present compound (such as the polypeptide with the general formula (I), the payload molecule, the linker, etc.) with each other to form a polypeptide with a single amino acid chain.

According to another preferred embodiment, the polypeptide with the general formula (I) is covalently coupled to the C-terminus of a payload molecule to be delivered into a biological cell which is a class II restriction endonuclease, preferably PvuII or a subunit thereof, wherein the payload molecule is optionally attached at its C-terminus to a linker molecule comprising 8 to 12 amino acids comprising or consisting of glycine, serine and/or cysteine residues, and wherein the polypeptide with the general formula (I) is covalently coupled to the C-terminus of the payload molecule or the linker molecule, wherein the compound comprises one or two payload molecule(s), optionally separated by the linker molecule comprising 8 to 12 amino acids comprising or consisting of glycine, serine and/or cysteine residues.

Preferably, the polypeptide with the general formula (I) is covalently coupled to the C-terminus of a payload molecule to be delivered into a biological cell, wherein the compound comprises a first payload molecule which is the first subunit of PvuII linked at its C-terminus to a linker molecule comprising 8 to 12 amino acids comprising or consisting of glycine, serine and/or cysteine residues, wherein the linker molecule is attached at its C-terminus to the second subunit of PvuII and wherein the polypeptide with the general formula (I) is covalently coupled to the C-terminus of the second subunit of PvuII. An example of such a compound according to the present invention may have the following structure (unless stated otherwise: always from N- to C-terminus): PvuII-1^{st} subunit-DDM. Another example is a compound having the following structure: PvuII-1^{st} subunit—8-12 amino acid linker comprising glycine, Serine and Cysteine residues—PvuII-2^{nd} subunit-DDM.

The PvuII restriction endonuclease is 157 amino acid residues long and has naturally a homodimeric form. The homodimer can easily be converted into a single polypeptide chain (sc PvuII). The two subunits may be tandemly linked through a short peptide linker (see above). The arrangement of a single-chain PvuII (sc PvuII) may be (2-157)-linker-(2-157), where (2-157) represents the amino acid residues of the enzyme subunit. PvuII endonuclease activity as sc enzyme may be expressed at high level as a soluble protein. The purified enzyme was shown to have the molecular mass expected for the designed sc protein. The cleavage specificity of the sc PvuII is indistinguishable from that of the wild-type (wt) enzyme.

These preferred CPP moieties according to the present invention (the “Drug Delivery Module” or “DDM” sequences) turned out to significantly to increase membrane crossing and desired intracellular sorting activities, increase stability towards proteolytic degradation, increase solubility of a given payload (especially a polypeptide payload) or the expression of a polypeptide encoded by a polynucleotide molecule (as payload) and simplify payload purification.

According to a preferred embodiment, the compound according to the present invention additionally comprise a payload molecule to be delivered into a biological cell covalently attached to the polypeptide with the general formula (I), preferably a chemotherapeutic molecule, a cytotoxic molecule, a DNA damaging molecule, an anti-metabolite molecule, a therapeutic molecule, a small molecule with therapeutic effect inside a biological cell, a DNA molecule, an RNA molecule, an antibody molecule or an antibody derivative having an antibody-like function, a restriction endonuclease, a nicking enzyme, or DNA- or RNA-dependent endonucleases which show double strand breaking nuclease double strand/single strand breaking activity or no nuclease activity, more preferred wherein the payload molecule is a class II restriction endonuclease, such as PvuII, EcoRV, PvuII, HinfI, or a sc homodimer, a subunit or a functional fragment thereof; especially wherein the payload molecule is selected from the group

(CP01)
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
(CP02)
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIY,
(CP36)
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGGSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
(CP60)
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIY,
(CP62)
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
(CP63)
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGCCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
(CP70)
SHPDLNKLLELWPHIQEYQDLALKHGINDIFQDNGGKLLQVLLITGLTVLPGREG-
NDAVDNAGQEYELASINIDLTKGESTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIY,
(CP87)
SHPDLNKLLELWPHIQEYQDLALKHGINDIFQDNGGKLLQVLLITGLTVLPGREG-
NDAVDNAGQEYELASINIDLTKGESTHHHMNPVIIAKYRQVPWIFAIYRGIAIEAIYRLEPKD-
LEFYYDKWERKWYSDGHKDINNPKIPVKYVMEHGTKIYGSGGCCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIY,
(CP88)
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
and
(CP89)
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIY .

According to a preferred embodiment, the compound according to the present invention additionally comprises a homopolymer moiety covalently attached to the polypeptide with the general formula (I). Although such homopolymer-containing CPP compounds are in principle identified in the prior art as being advantageous to enhance CPP function in a druggable context (s. e.g. WO 2020/214846 A1 and the further documents cited in the search report to this international application), this is challenged by the resulting size of the molecule due to modifications with polymers and endosome trafficking. Moreover, the use of polymer therapeutics was reported to be hindered, because the cell membrane barrier often impedes their intracellular delivery.

The CPP moieties in the compound according to the present invention turned out to be so powerful in delivering payload through the cell barriers, the addition of polymers, especially homopolymers, can be foreseen in the compounds of the present invention so that the advantages accepted in the present field for such polymers attached to CPPs, including the ability of a certain compound to be adjusted with respect to the pharmaco-kinetic (PK) and pharmaco-dynamic (PD) properties of a given payload, are present for the compounds according to the present invention without the drawbacks of the enlarged size, the low cell type selectivity and the overall equal tissue distribution. Preferably, the homopolymer of the compound according to the present invention is selected from the group of polyethylene glycol (PEG), especially a PEG with a MW of 5 to 30 kDa; dextran, polysialic acids, hyaluronic acid, dextrin, hydroxyethyl starch, poly(2-ethyl 2-oxazoline, etc. (Pasut, Polymers 6 (2014), 160-178; Grigoletto et al., Nanomed. Nanobiotechnol. 2020, e1689); alternatively, also polypeptide techniques, such as protein conjugates with XTEN peptides or PASylation are available.

All polymer therapeutics used in practically relevant therapeutic applications are water soluble polymers. One of the most commonly employed polymers in polymer therapeutics is PEG, also specifically preferred for the present invention. PEG is approved for human administration by various routes of administration, e.g. mouth, injection, or dermal application. The structure of linear PEG is HO—(CH₂—CH₂—O)ₙ—H, where n indicates the number of repeats of the ethylene oxide unit in the PEG. PEG is a linear or branched, neutral polyether, and is commercially available in a variety of molecular mass; the polymerization can be controlled such that the molecular mass distribution is narrow. PEG derivatives can have different sizes with typically molecular weights ranging from hundreds to thousands of Da. PEG molecules used for polymer molecules are preferentially highly purified molecules with little or no diol impurities. Preferred PEG moieties used for the invention are mono-disperse or show only narrow molecular mass range differences (+/−5% or better+/−3%). Many PEG derivatives are available on the market or can be produced with known methods in the art.

The present invention therefore preferably relates to a PEGylated compound. Many of the benefits of PEGylated therapeutics lie in the properties of PEGs. PEGs are neutral, hydrophilic polymers that are soluble in water and a variety of organic solvents. Further, PEGs are inert, non-toxic, have a low immunogenicity, and the polymer is easily cleared from the body, mainly through the kidney for molecules with a molecular mass below 20 kDa, or through a combination of kidney and liver for molecules with molecular mass above 20 kDa. Up to day, the maximum PEG molecular mass used for the preparation of polymer therapeutics is 40 kDa. PEGylation plays an important role in the stabilization of drugs, reduction of their antigenicity and decrease in the drug doses, besides augmenting the biodistribution ability via binding biologics onto their surfaces. PEGs possess the most suitable quality for preparation of physiologically active drug and biologics. Various functional groups are available and enable the introduction of the PEG chains into drugs, enzymes, phospholipids and other biologics. Covalent conjugation of hydrophobic macromolecules with activated PEGs leads to the formation of macromolecular micelles, which allow homogeneous dispersion of hydrophobic drugs in aqueous media.

Coupling of PEG or PEG derivatives to polypeptides (such as the polypeptide with the general formula (I)) can be obtained by coupling of PEG-NHS derivatives to polypeptide amines (PEG-NHS+polypeptide-NH₂) such as epsilon groups of lysine, coupling of PEGaldehyde derivatives to the NH₂ group of polypeptides (PEG-Ald+polypeptide-NH₂), including secondary amines such as the N-terminus of a polypeptide, frequently used aldehyde linker include methoxy-PEG-CO(CH₂)nCOO—NHS, whereas n represents an integer of 1 to 3; or coupling of PEG-maleimide derivatives to the SH-group of polypeptide (PEG-Maleimide+polypeptide-SH) such as coupling to exposed cysteines contained within a polypeptide or engineered to the polypeptide by recombinant techniques, or coupling of PEG-NH₂ derivatives to the COOH group of polypeptides (PEG-NH₂+polypeptide-COOH) such as surface exposed glutamic acid of a polypeptide or coupling of PEG-p-nitrophenyloxycarbonyl derivatives to the NH₂ group of polypeptides (PEG-NP+polypeptide-NH₂).

PEG derivatives can be a mono-functional linear PEGs, such as NHS active esters/carbonate, p-nitrophenyl carbonate PEG aldehyde PEG, aminopropyl PEG, aminoethyl PEG, thiol PEG, maleimide PEG, aminoxy PEG, hydrazide PEG, iodoacetamide PEG; or a bi-functional PEG, such as NHS-PEG, amine PEG, thiol PEG, maleimide PEG, or a multi-arm PEGs such as a 4-arm-PEG or an 8-arm-PEG; or a branched PEG such as a 2-arm branched PEG, 3-arm branched PEG, 4-arm branched PEG or a lysine branched PEG; or a heterofunctional PEG, such as Boc-protected-amino-PEG-carboxylic acid, 9-fluorenylmethyloxycarbonyl-protected-amino-PEG-carboxylic acid, maleimide PEG-carboxylic acid, maleimide-PEG-NHS ester/carbonate, amino PEG-carboxylic acid, Boc-protected-amino PEG-NHS carbonate, protected-mercapto-PEG NHS ester, 9-flourenylmethyloxycarbonyl-protected-amino-PEG-NHS ester, azido-PEG-NHS ester/carbonate, azido PEG-amine, Biotin-PEG-NHS ester/carbonate, Biotin-PEG-maleimide, Biotin-PEG-amine; or also a forked PEG such as modified as a NHSPEG, amine PEG or a maleimide PEG. A variety of PEG derivatives has been developed for such applications. Most polymer-macromolecules are chemical synthesized and can be coupled using linker technology known in the art to virtually all the known classes of therapeutically suitable molecules such as nucleic acids, PNA, lipids, small molecules peptides or proteins or mixtures thereof. Most of these molecules and in particular molecules with an extending molecular mass of more than >1000 Da are at least in part synthesized with molecular recombinant methods and it would be of advantage to have a polymer-macromolecule at disposal that can be produced recombinant as well including those polymers that can be produced as covalent fusion allowing for single step expression. Overall, covalent conjugation of polymers, e.g. PEG, with small molecule drugs and/or biological macromolecules such as polypeptides or polynucleotides is a promising approach for pharmaceutical applications, since such conjugates display altered (improved) pharmacokinetic properties, including a longer in vivo half-life; reduced immunogenicity; reduced toxicity; protection against proteolysis; improved water solubility; and increased pH and thermal stability; while the biological activity of the small molecule drug and/or the biological macromolecule is commonly retained in those conjugates.

The compounds according to the present invention do not show undesirable off target effects and do not require frequently high therapeutic dosing, as has been reported for prior art polymer-CPP compounds.

In fact, the compounds according to the present invention generally show sufficient and often advantageous target specificity in selecting target selective active principles. The compounds according to the invention help to increase localized tissue distribution and to obtain selective cell type specificity. Thus, the compounds according to the present invention provide better target specificity to therapeutics, especially also to polymeric therapeutics. Such polymeric therapeutics encompass polymer-macromolecule conjugates, drug-polymer conjugates, and supramolecular drug-delivery systems. Besides a favourable bio-distribution, these polymer therapeutics can accomplish several further objectives to optimize for CPP activity and adapt the pharmacokinetics profile of a polymer.

The combination of the new CPP moieties provided with the present invention together with polymer-modified macromolecular single activity profile compounds, or CPPs together with polymer-modified macromolecular multiple activity profile compounds can be engineered to be sorted to specific locations (specific bio-distribution) after systemic treatment in vivo. Moreover, such compounds (even with a polymeric moiety attached) show efficient cross membrane passing by receptor mediated energy dependent endocytosis pathways. For designing the compounds according to the present invention, it turned out to be advantageous (and often compulsory for effective transfer of many payload molecules) to not attach the CPP via a polymeric moiety to the payload molecule but to link the polymeric moiety and the payload molecule to different amino acid residues of the CPP moiety of the compound according to the present invention.

According to a preferred embodiment, the compound according to the present invention additionally comprises a restriction endonuclease as a payload molecule to be delivered into a biological cell covalently attached to the polypeptide with the general formula (I), preferably a class II restriction endonuclease, especially a PvuII restriction endonuclease or a derivative thereof wherein the derivative is a single chain PvuII restriction endonuclease, such as the derivative with the amino acid sequence

SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGG,
SGGSSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGNSG,
MSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGC,
CSGGSSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
MSHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELKSINIDLTKGESTHHHMNPVIIAKYRQVPWIFAIYRGIAIEAI-
YRLEPKDLEFYYDKWERKWYSDGHKDINNPKIPVKYVMEHGTKIY,
MSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
MSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGSGGS,
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGSGGS,
or
GSGGSGGSSHPDLNKLLELWPHIQEYQDLALKHGIN-
DIFQDNGGKLLQVLLITGLTVLPGREGNDAVDNAGQEYELKSINIDLTKGFSTHHHMNPVIIA-
KYRQVPWIFAIYRGIAIEAIYRLEPKDLEFYYDKWERKWYSDGHKDINNPKIPVKYVME-
HGTKIY.

Therefore, a specifically preferred embodiment of the compound of the present comprises as payload two monomeric subunits or parts thereof of a type II restriction endonuclease. Preferably, two monomeric alpha-helical subunits of a type II restriction endonuclease can be covalently connected by a linker. Further preferred, the two monomeric alpha-helical subunits can be subunits of PvuII. Specifically preferred is a compound comprising two monomeric subunits or parts thereof of the type II restriction endonuclease PvuII covalently connected by a linker to the CPP moiety of the compound of the present invention. The linker is preferably an amino acid linker of 1 to 20 amino acid residues in length, preferably from 1 to 15 amino acid residues, more preferred from 7 to 13 amino acid residues, especially of 8 to 12 amino acid residues. Preferred amino acid linkers are linkers comprising or consisting of a cysteine, serine or glycine residue (or two, three four or five consecutive cysteine or glycine residues), especially a single or two adjacent cysteine residue(s); or an amino acid linker comprising glycine and serine residues or comprising or consisting of the amino acid sequences GSG, SGG, GGC, GCS, CSG, GGS, GSGG, SGGS, GSGGC, CSGGS, GSGGSGGS, GSGGCCSGGS, GSGGCCCSGGS, or GSGGCSGGS.

Type II restriction endonucleases are reviewed e.g. by Pingoud et al. (NAR 29 (2001), 3705-3727; and NAR 42 (2014), 7489-7527); the common structure and function of type II restriction endonucleases is therefore well available to the person skilled in the art. A “derivative” of a restriction endonuclease is a polypeptide which is modified compared to the wild-type restriction endonuclease but still comprises the main function of the wild-type restriction endonuclease, i.e. the specific cleavage property, i.e. the ability to cut a nucleic acid molecule at a sequence-specific site. For the preferred example of PvuII, this means that a PvuII derivative is a derivative of the wild-type PvuII polypeptide which is modified compared to the wt PvuII polypeptide but still comprises the function of wild-type PvuII to specifically recognising the double-stranded DNA sequence 5′-CAGCTG-3′ and cleave after G-3 (Cheng et al., EMBO J. 13 (1994), 3927-3935).

According to a preferred embodiment, the polypeptide with the general formula (I) in the compound of the present invention is covalently linked to another moiety by a linker, wherein the another moiety is preferably a payload molecule to be delivered into a biological cell, a labelling group, and/or a homopolymer.

A preferred embodiment is a compound according to the present invention, wherein the compound further comprises a capping group, preferably an alkoxy, especially a methoxy, ethoxy, propoxy, or butoxy group; a halogen atom; or a tosylate; isocyanate, hydrazine hydrate, maleimide, orthopyridyl disulfide, N-succinimidyloxy, sulfo-N-succinimidyloxy, 1-benzotriazol, 1-imidazolyloxy, p-nitrophenyloxy, or aldehyde.

Another aspect of the present invention is drawn to the compound according to the present invention, for use in the treatment of a tumour patient, preferably wherein the tumour patient is suffering from neuroblastoma, colon carcinoma, hepatocellular carcinoma, Small Cell Lung carcinoma, ovarian carcinoma, lung carcinoma, bladder carcinoma, prostate carcinoma, uterus carcinoma, cervix carcinoma, placental carcinoma, breast carcinoma, kidney carcinoma, liver carcinoma, pancreas carcinoma, muscle carcinoma, skin carcinoma, connective tissue carcinoma, bone carcinoma, and any of these carcinomas wherein the patient has already developed metastases, especially for the treatment of a patient having neuroblastoma, colon carcinoma, hepatocellular (liver) carcinoma, Small Cell Lung carcinoma, lung carcinoma, breast carcinoma, pancreas carcinoma, and any of these carcinomas wherein the patient has already developed metastases.

Another aspect of the present invention is drawn to a method of treatment of a tumour patient wherein an effective amount of the compound according to the present invention is administered to a patient in need thereof, preferably wherein the tumour patient is suffering from neuroblastoma, colon carcinoma, hepatocellular carcinoma, Small Cell Lung carcinoma, ovarian carcinoma, lung carcinoma, bladder carcinoma, prostate carcinoma, uterus carcinoma, cervix carcinoma, placental carcinoma, breast carcinoma, kidney carcinoma, liver carcinoma, pancreas carcinoma, muscle carcinoma, skin carcinoma, connective tissue carcinoma, bone carcinoma, and any of these carcinomas wherein the patient has already developed metastases, especially for the treatment of a patient having neuroblastoma, colon carcinoma, hepatocellular (liver) carcinoma, Small Cell Lung carcinoma, lung carcinoma, breast carcinoma, pancreas carcinoma, and any of these carcinomas wherein the patient has already developed metastases.

Another aspect of the present invention is drawn to the use of a compound according to the present invention for the manufacture of a medicament, preferably for the treatment of a tumour patient, more preferred for the treatment of a patient having neuroblastoma, colon carcinoma, hepatocellular carcinoma, Small Cell Lung carcinoma, ovarian carcinoma, lung carcinoma, bladder carcinoma, prostate carcinoma, uterus carcinoma, cervix carcinoma, placental carcinoma, breast carcinoma, kidney carcinoma, liver carcinoma, pancreas carcinoma, muscle carcinoma, skin carcinoma, connective tissue carcinoma, bone carcinoma, and any of these carcinomas wherein the patient has already developed metastases, especially for the treatment of a patient having neuroblastoma, colon carcinoma, hepatocellular (liver) carcinoma, Small Cell Lung carcinoma, lung carcinoma, breast carcinoma, pancreas carcinoma, and any of these carcinomas wherein the patient has already developed metastases.

A “CPP” as used herein means an amino-acid sequence, or polynucleotide encoding the same, which facilitates active transport of a biological macro-molecule across a biological membrane or a physiological barrier. Transduction across a biological membrane or a physiological barrier can be determined by various processes, for example by a cell penetration test having a first incubation step for the PTD conjugated to a marker in the presence of culture cells, followed by a fixating step, and then detection of the presence of the marked peptide inside the cell. Preferably, the CPP activity of a given molecule is tested according to the model system disclosed in the example section using the CROMOC molecule as a payload model. Alternatively, detection can be accomplished with an incubation of the CPP in the presence of labelled antibodies and directed against the CPP, followed by detection in the cytoplasm or in immediate proximity of the cell nucleus, or even within it, of the immunologic reaction between the CPP's amino acid sequence and the labelled antibodies. Cell penetration tests are well known to those skilled in the art.

The term “capping group” as used herein means any suitable chemical group which, depending upon preference, is unreactive or reactive with other chemical moieties. Accordingly, the capping group is selected to provide monofunctionally, i.e. the terminal aldehyde group, or bifunctionality, i.e. an aldehyde group on one terminus and a different functional moiety on the opposite terminus. If the capping group is unreactive with other chemical moieties, then the structure of the resulting polymer aldehyde derivative is monofunctional and therefore can covalently bond with only one chemical moiety of interest. In other words, in the case that the capping group is unreactive, the terminal aldehyde group of the compound of the present invention permits ready covalent attachment to a chemical moiety of interest, for example, to the α-amino group of a polypeptide. Suitable capping groups are generally known in the art, for example, those disclosed in WO 2004/013205. Suitable non re-active capping groups include, for example, alkoxy, e.g. methoxy, ethoxy, propoxy, or butoxy; halogen atom (i.e. fluorine, chlorine, bromine, or iodine atom); or tosylate; and suitable reactive capping groups include, for example, isocyanate, hydrazine hydrate, maleimide, orthopyridyl disulfide, N-succinimidyloxy, sulfo-N-succinimidyloxy, 1-benzotriazol, 1-imidazolyloxy, p-nitrophenyloxy, or aldehydes.

The terms “group”, “functional group”, “moiety”, “active moiety”, “active compound”, “effective compound”, “target”, “target molecule”, “radical”, etc. are somewhat synonymous in the chemical arts and are used in the art and herein to refer to distinct, definable portions or units of the compound according to the present invention and to units that perform some function or activity and are reactive with other molecules or portions of molecules. In this sense a small molecule drug or a polypeptide residue can be considered a functional group or moiety when coupled to a compound of the present invention.

The term “small molecule drug” or “small molecule” as used herein preferably refers to a medicinal organic compound having a molecular mass of less than 1000 Da, typically of 300 to 1000 Da, and preferably of 300 to 700 Da. The small molecule drug activates or inhibits the function of a biomolecule which in turn results in a therapeutic benefit to a patient, e.g. a mammal, preferably a human. This is, the small molecule drug usually binds with high affinity to a biomolecule such as a polypeptide, nucleic acid, or polysaccharide and alters the activity or function of the biomolecule. The small molecule drug can be natural compounds (such as secondary metabolites, including alkaloids, terpenoids, steroids, glycosides, natural phenols, phenazines, polyketides, fatty acid synthase products, non-ribosomal peptides, macrolactones, and polyphenols) or artificial counterparts thereof.

The preferred small molecule drugs for use in the present invention are cytotoxic agents, particularly those which are used for cancer therapy. Such drugs include, in general, DNA damaging agents, anti-metabolites, natural products and their analogs.

Preferred classes of cytotoxic agents include, for example, natural or synthetic tubulysins; natural or synthetic epothilones; duocarmycines; auristatins; maytansinoids; calicheamycin; mesothelins; anthracyclins; dihydrofolate reductase inhibitors; and thymidylate synthase inhibitors; DNA intercalators; DNA cleavers; topoisomerase inhibitors; the vinca drugs; the mitomycins; the bleomycins; the cytotoxic nucleosides; the pteridine family of drugs; diynenes; the podophyllotoxins; differentiation inducers; and taxanes. Useful members of those classes include, for example, erlotinib (TARCEVAR®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®), Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin. Also useful cytotoxic agents include: oxaliplatin (ELOXATIN®, Sanofi), bortezomib (VELCADE®, Millennium Pharm.), sutent (SUNITINIB®, SU1 1248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1 126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), vandetanib (rINN, ZD6474, ZACTIMA®, AstraZeneca), chlorambucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gamma 1, calicheamicin omega 11 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and known functional derivatives thereof.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, m-RNA vaccines, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); leurosine, carminomycin, tallysomycin, podophyllotoxin, retinoic acid, butyric acid, N⁸-acetyl spermidine, and pharmaceutically acceptable salts, acids and any known functional analogues thereof.

The term “labeling group” as used herein refers to a label, or tag, or tracer, or marker, i.e. an easily recognizable chemical moiety, which allows to follow the translocation and/or the chemical or biological transformation of the moiety of interest, e.g. of a conjugate formed with the compound of the present invention. Suitable labeling groups include, for example, affinity labels, including antibodies and antibody fragments, radioactive labels, and fluorophores. Particularly useful examples of a labeling group include: fluorescein, Cy5.5 and related cyanins emitting in the near UV.

The term “biological macromolecule” as used herein refers to any macromolecule that is produced by a living organism, including large polymeric molecules such as polypeptides (which includes the term “protein”), including antibodies, enzymes, polypeptides involved in the process of cell signaling and signal transduction, and membrane polypeptides that act as receptors, polysaccharides, lipids, nucleic acids and polynucleotides as well as primary metabolites, secondary metabolites, and natural products. Preferably, the “biological macromolecule” is a polypeptide or a polynucleotide.

The terms “polypeptide”, “protein”, “peptide” as used herein define an organic compound made of two or more amino acid residues arranged in a linear chain, wherein the individual amino acids in the organic compound are linked by peptide bonds, i.e. an amide bond formed between adjacent amino acid residues. By convention, the primary structure of a polypeptide is reported starting from the amino-terminal (N) end to the carboxyl-terminal (C) end.

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity. Antibodies may be murine, human, humanized, chimeric, or derived from other species. An antibody is a polypeptide generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species, including human, murine, or rabbit origin.

“Antibody fragments” comprise a portion of a full-length antibody, generally the antigen binding or variable region thereof. Examples of antibody fragments include Fab, Fab′, F(ab′)₂, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method or may be made by recombinant DNA methods. The monoclonal antibodies may also be isolated from phage antibody libraries using established techniques. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies include “primatized” antibodies comprising variable domain antigen-binding sequences derived from a non-human primate (e.g., Old World Monkey or Ape) and human constant region sequences.

An “intact antibody” herein is one comprising a VL and VH domains, as well as a light chain constant domain (CL) and heavy chain constant domains, CH1, CH2 and CH3. The constant domains may be native sequence constant domains (e.g., human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR. Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, β, ε, γ, and μ respectively. The subunit structures and three-dimensional configurations of different classes of immunoglobulins are well known.

Particularly useful antibodies are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG™, 2C4, Genentech), trastuzumab (HERCEPTIN®, Genentech), tositumomab (Bexxar, Corixia), and the humanized monoclonal antibodies alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

The term “amino acid” as used herein refers to any organic acid containing one or more amino substituents, e.g. α-, β- or γ-amino, derivatives of aliphatic carboxylic acids. Preferably, the term “amino acid” refers to the 22 most common, natural L-amino acids, which are selected from the group consisting of glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine, histidine, selenocysteine, and pyrrolysine. Of these, the 20 amino acids encoded by the universal genetic code can herein also be referred to by their conventional three-letter or one-letter abbreviations and their abbreviations follow conventional usage The remaining 2, selenocysteine and pyrrolysine, are incorporated into polypeptides by unique synthetic mechanisms. Unless otherwise and explicitly stated herein, all references to “amino acids” or “amino acid residues” shall refer to the 20 natural L-amino acids encoded by the genetic code.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; and (iii) unnatural amino acids, beta-amino acids, synthetic analogs and derivatives of naturally occurring amino acids.

The terms “nucleic acid”, “nucleic acid sequence”, “oligonucleotide”, “nucleotide”, or “polynucleotide” as used herein refer to DNA or RNA (e.g., mRNA, rRNA, tRNA, iRNA, gRNA, crRNA, traceRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., e.g., double stranded iRNAs, e.g., iRNPs).

These terms encompass natural and synthetic nucleic acids, including known analogues of natural nucleotides. The term also encompasses nucleic-acid-like structures with synthetic backbones. “Oligonucleotide” includes either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide can ligate to a fragment that has not been dephosphorylated. A “coding sequence of” or a “nucleotide sequence encoding” a particular polypeptide, is a nucleic acid sequence which is transcribed and translated into a polypeptide when placed under the control of appropriate regulatory sequences. The nucleic acids used to practice this invention may be isolated from a variety of sources, genetically engineered, amplified, and/or expressed/generated recombinantly. Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature. A nucleic acid encoding a polypeptide of the invention is assembled in appropriate phase with a leader sequence capable of directing secretion of the translated polypeptide or fragment thereof.

The compounds referred to herein are meant and claimed as “isolated” and/or “purified” and/or “synthetic” compounds. The term “isolated” as used herein means that the compound is removed from any natural environment, e.g., if it (or part of it) is naturally occurring. The term “purified” does not require absolute purity; rather, it is intended as a relative definition. The term “synthetic” as used herein means that the compound does not occur in nature and has been synthesized in vitro by well-known chemical or biological (recombinant) synthesis techniques. The terms “recombinant” or “chimeric” mean that at least two functional moieties, which are not linked, combined or present in their natural environment, are linked to each other to thereby provide a novel not naturally occurring combination of said functional moieties, e. g. a polypeptide domain from one protein that is linked to a polypeptide domain from a second, different and unrelated protein. Recombinant polypeptides can be generated from nucleic acids encoding them by expression in a suitable expression system and further purified from this expression system, and further individually isolated or cloned and tested for a desired activity. Any recombinant expression system can be used, including bacterial, mammalian, yeast, insect or plant cell expression systems.

The present compound preferably further comprises a restriction endonuclease or a nicking enzyme as payload.

Preferred restriction endonuclease are type II restriction endonucleases, especially selected from AatII, AbaSI, Acc65I, AccI, AciI, AclI, AcuI, AfeI, AflII, AflIII, AgeI, AhdI, AleI, AluI, AlwI, AlwNI, ApaI, ApaLI, ApeKI, ApoI, AscI, AseI, AsiSI, AvaI, AvaII, BaeGI, BaeI, BamHI, BanI, BanII, BbsI, BbvCI, BbvI, BccI, BceAI, BcgI, BciVI, BclI, BcoDI, BfaI, BfuAI, BfuCI, BglI, BglII, BlpI, BmgBI, BmrI, BmtI, BpmI, BpulOI, BpuEI, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFI, BsrGI, BsrI, BssHII, BssKI, BssSaI, BstAPI, BstBI, BstEII, Bst, BstUI, BstXI, BstYI, BstZ17I, Bsu36I, BtgI, BtgZI, BtsaI, BtsCI, Cac8I, ClaI, CspCI, CviAII, CviKI-1, CviQI, DdeI, DpnI, DpnII, DraI, DraIII, DrdI, EaeI, EagI, EarI, EciI, Eco53kI, EcoNI, EcoO109I, EcoP15I, EcoRI, EcoRV, FatI, FauI, Fnu4HI, FokI, FseI, FspEI, FspI, HaeIII, HgaI, HhaI, HincII, HindIII, HinfI, HinPlI, HpaI, HpaII, HphI, Hpyl66II, Hpyl88I, Hpyl88III, Hpy99I, HpyAV, HpyCH4III, HpyCH4IV, HpyCH4V, I-CeuI, I-SceI, KasI, KpnI, LpnPI, MboI, MboII, MfeI, MluCI, MluI, MlyI, MmeI, MnlI, MscI, MseI, MslI, MspAlI, MspI, MspJI, MwoI, NaeI, NarI, Nb.BbvCI, Nb.BsmI, Nb.BsrDI, Nb.BtsI, NciI, NcoI, NdeI, NgoMIV, NheI, NlaIII, NlaIV, NmeAIII, NotI, NruI, NsiI, NspI, Nt.AlwI, Nt.BbvCI, Nt.BsmAI, Nt.BspQI, Nt.BstNBI, Nt.CviPII, PacI, PaeR7I, PciI, PflFI, PflMI, PI-PspI, PI-SceI, PleI, PluTI, PmeI, PmlI, PpuMI, PshAI, PsiI, PspGI, PspOMI, PspXI, PstI, PvuI, PvuII, RsaI, RsrII, Sac, SacII, SalI, SapI, Sau3AI, Sau96I, SbfI, ScaI, ScaI, ScrFI, SexAI, SfaNI, SfcI, SfiI, SfoI, SgrAI, SmaI, SmlI, SnaBI, SpeI, SphI, SspI, StuI, StyD4I, StyI, SwaI, TaqaI, TfiI, TseI, Tsp45I, TspMI, TspRI, Tth111I, XbaI, XcmI, XhoI, XhoI, XmaI, XmnI, and ZraI; as well as any nicking variants thereof or versions thereof with no nuclease activity (Sequence-specific DNA nicking endonucleases. Shuang-yong Xu, Biomolecular Concepts, De Gruyter Aug. 7, 2015).

Further preferred payloads (especially polypeptidic payloads) are selected from zinc finger nucleases (ZFNs), zinc finger transcription activators (ZFAs), zinc finger transcription repressors (ZFRs) and zinc finger methylases (ZFMs); enzymes in disease for enzyme-replacement therapy, such as glucose-6-phosphate dehydrogenase (EC 1.1.1.49) (G6PD), 21-hydroxylase (EC 1.14.99.10), steroid 11β-monooxygenase (EC 1.14.15.4), 3α-hydroxysteroid-3-dehydrogenase (EC 1.1.1.50), Steroid 17α-monooxygenase (EC 1.14.99.9), cholesterol monooxygenase (EC1.14.15.6)), Glucosylceramidase (EC 3.2.1.45), galactosylceramidase (EC 3.2.1.46), Pyruvate kinase (EC 2.7.1.40), α-galactosidase(EC3.2.1.22), Bgalactosidase (EC3.2.1.23)acid α-glucosidase (GAA), α-Amylase (EC 3.2.1.1), β-amylase (EC 3.2.1.2.), collagenase (EC 3.4.24.7), glutamina-se(EC 3.5.1.2), asparaginase (EC 3.5.1.1), lysozyme (EC 3.2.1.17), bilirubin oxydase (EC 1.3.3.5), Thiosulfate sulfur-transferase (EC 2.8.1.1), β-lactamase (EC 3.5.2.6),streptokinase, urokinase (EC 3.4.21.73), hyalurono-glucosaminidase (EC 3.2.1.35), hyalurono-glucuronidase (EC 3.2.1.36), hyaluronate lyase (EC 4.2.2.1), Superoxide dismutase (EC 1.15.1.1), urease (EC 3.5.1.5), arginase (EC 3.5.3.1), glutamyl-transferase (EC 2.3.2.2.), glutathione-S-transferase (EC 6.3.1.13), creatine kinase (EC 2.7.3.2), aldolase (EC 4.1.2.13), lipase (EC 3.1.1.79), hydroxymethylglutaryl-CoA reductase (EC 1.1.1.34), Xantin oxydase, PRPP Synthase (EC 2.7.6.1), hypoxantine-guanine-Phosphoribosyltransferase (EC 2.4.2.8), DNAse (EC 3.1.22.1), RNAse (EC 3.1.27.5), Branched-chain ketoacid dehydrogenase (EC 1.2.4.4), Adenosine deaminase (EC 3.5.4.4), hexoseaminidase (EC 3.2.1.52), R-galactosidase (EC 3.2.1.23), sphingomyelinase(EC 3.1.4.12), ceramidase (EC 3.5.1.23), homogenitisate-oxydase, R-glucosidase (EC 3.2.1.21), lactase (EC 3.2.1.108), HIV-protease (EC 3.4.23.16), dihydro-folate reductase (EC 1.5.1.3), alcohol-dehydrogenase (EC 1.1.1.2), Iduronate-2-sulfatase (EC 3.1.6.13), or sphingomyelin phosphodiesterase (EC 3.1.4.12).

A ZFN is achieved by linking several zink finger (ZF) motifs in tandem to form ZFPs (zinc finger proteins). To the designed ZFPs other functionalities like non-specific FokI cleavage domain (N, nuclease), transcription activator domains (A), transcription repressor domains (R) and methylases (M) are fused to a ZFPs to form ZFNs respectively.

The compounds according to the present invention can also comprise editing nucleases, CRISPR nucleases and hybrids thereof. The CRISPR-associated protein Cas9 is an RNA-guided endonuclease that cleaves double-stranded DNA bearing sequences complementary to a 20-nucleotide segment in the guide RNA. Cas9 and its orthologues has emerged as a versatile molecular tool for genome editing and gene expression control. RNA-guided DNA recognition and cleavage strictly require the presence of a protospacer adjacent motif (PAM) in the target DNA. A HNH-motif (His-Asn-His) nuclease like domain in Cas9 cleaves the DNA strand complementary to the RNA guide and RuvC endonuclease like domain initiates cleavage of the non-complementary DNA strand to the guide RNA respectively resulting in a blunt end DNA double strand break between the phosphates of +3 and +4 upstream of the PAM sequences. This clearly separable molecular features are used in the art to engineer single strand cutter (or nickase) or catalytic inactive enzymes which still show guide RNA dependend binding selectivities. Along these lines, single point mutations of H840A, N863A, N854A or D839A or combinations thereof results in an inactive HNH nuclease cleavage, mutation of any of the catalytic amino acids H983A, D986A, D10A, or E762A—results in loss-of-function of the RuvC nuclease like activity and therefore produce Cas9 enzymes that can be used as a single strand cutter or nickases. Loss of nuclease function of Cas 9 is obtained from combinations of HNH and RuvC nuclease null mutations in a single Cas9 poly-peptide for example D10A, D839A, H840A, N863A. These variations of CRISPR nucleases can be produced as polypeptides fused to the polypeptide with the general formula (I) in a similar way as exemplified for Cas9-NLS-DDM an example for an expression construct for a HNH mutated nicknase, a RuvC mutated nicknase or a Cas9 null nuclease. This is also applicable for non-natural editing enzymes. In the present invention, “Cas9” represents any type of functional Cas9 polypeptide nuclease enzyme with or without one or more guide RNA (gRNAs) including those Cas9 enzymes with nicking activity or with nuclease impaired but DNA binding activity; the sequences for designing Cas9 molecules are selected from those genera that contain Type II CRISPR systems such as for example from: Acetobacter, Acholeplasma, Acidaminococcus, Acidovorax, Actinobacillus, Afipia, Alicycliphilus, Alistipes, Aminomonas, Anaerococcus, Arthrobacter, Atopobium, Bacillus, Bacteroidales, Bacteroides, Bacteroidetes, Barnesiella, Bdellovibrio, Belliella, Bergeyella, Bibersteinia, Bifidobacterium,Blastopirellula, Bordetella, Bradyrhizobium, Brevibacillus, Brochothrix, Burkholderiales, Butyrivibrio, Campylobacte, Capnocytophaga, Catellicoccus, Catenibacterium, Chryseobacterium, Clostridiales, Clostridium, Collinsella, Coprobacillus, Coriobacteriaceae, Corynebacterium, Cryptobacterium, Cryptococcus, Cyclobacteriaceae, Defluviimonas, Dialister, Dinoroseobacter, Dolosigranulum, Dorea, Eggerthella, Elizabethkingia, Elusimicrobium, Enterococcus, Escherichia, Eubacterium, Facklamia, Fibrobacter, Filifactor, Finegoldia, Firmicutes, Flavobacterium, Fluviicola taffensis, Francisella, Francisella, Fusobacterium, Galbibacter, Gemella, Geobacillus, Gluconacetobacter, Gordonibacter, Gracilibacillus, Granulibacter, Haemophilus, Halobacillus, Helcococcus, Helicobacter, Hyphomonas, Ignavibacterium, Ilyobacter, Indibacter, Joostella, Kingella, Kordia, Lachnospiraceae, Lactobacillus, Legionella, Leptotrichia, Leuconostoc, Leuconostoc, Listeria, Mannheimia, Maritimibacter, Marmosops, Megasphaera, Mollicutes, Mucilaginibacter, Mucinivorans, Mucispirillum, Mycoplasma, Myroides, Narcissus, Negativicoccus, Neisseria, Niabella, Nitratifractor, Nitrobacter, Nitrosomonas, Novosphingobium, Oceanobacillus, Odoribacter, Oenococcus, Oligella, Olsenella, Oribacterium, Ornithobacterium, Parabacteroides, Parasutterella, Parvibaculum, Parvimonas, Pasteurella, Pasteurella, Pediococcus, Pedobacter, Peptoniphilus, Peptostreptococcaceae, Phascolarctobacterium, Planococcus, Porphyromonadaceae, Porphyromonas, Prevotella, Proteobacterium, Pseudoramibacter, Psychroflexus, Puniceispirillum, Ralstonia, Rhodopseudomonas, Rhodospirillum, Rhodovulum, Riemerella, Roseburia, Ruminococcus, Saccharibacter, Salinispira, Scardovia, Simonsiella, Solobacterium, Sphaerochaeta, Sphingobacterium, Sphingobium, Sphingomonas, Spiroplasma, Sporocytophaga, Staphylococcus, Streptobacillus, Streptococcus, Subdoligranulum, Succinatimonas, Sulfitobacter, Sulfurimonas, Sulfuritalea, Sulfurospirillum, Sulfurovum, Sutterella, Tannerella, Tistrella, Treponema, Veillonella, Verminephrobacter, Weeksella, Wolinella, Zunongwangia. Based on published Cas9 sequences from these generas (UNIprot-Expasy) the corresponding DNA can be easily synthesized by synthetic DNA techniques well described in the field or by PCR cloning from the respective hosts. Cas9 represents also chimeras were the RNA binding and targeting features are used form Cas9 like molecules and the nucleases are supplemented or exchanged by other enzymatic features such as Nucleic acid modifying enzymatic activities, methylases, acetylases, deacetylases, polymerases, reverse transcriptases, RNases, Rnases H; Helicases, recA like activities, topoisomerase, transcription Factors, -activators, transcription repressors, ubiquitin and sumo ligases, debuiquitinases. “gRNA” is the mature crRNA:tracrRNA complex or a truncated RNA chimera containing a designed hairpin of these is used. These RNAs can be ideally obtained by chemical synthesis or in vitro transcription protocols well known in the art. The chemical synthesis offers in addition the possibility of the use of gRNAs containing non-natural RNA nucleotides that provides additional advantages for the use of Cas9-CPP polypeptide RNA complexes as a medicine. Examples for modified nucleotides are a partial or complete use of chemical modified RNA such as for example nucleotides modified as phosphothioate (PS), locked nucleic acid (LNA) 2′-O-methoxyethyl (MOE), 2′-O-methyl (OMe) or 2′-fluoro (2′-F). These compounds according to the present invention can be applied for the editing of genetic information in vitro, ex vivo and in vivo. For this the compounds according to the present invention are directly applied to the cell-culture-medium or buffer of a cell culture for in vitro or ex vivo applications, or are directly injected into the bloodstream or by subcutaneous application of specific Cas9-CPP compounds in the full body context for in vivo application. These compounds according to the present invention can be used in particular for gain of function editing by homologous recombination with the nucleic acid containing compound as template for recombination and the Cas9 nuclease to introduce a site directed double strand break near the mutation. This mixture can be used to introduce a site directed genetic change useful to introduce a site directed mutation including for the repair of a genetic defect.

The Cas9-CPP polypeptides and Cas9-CPP:RNA complexes according to the present inventio may be obtained, a) by recombinant techniques, expressing either the Cas9-CPP molecules, and subsequent purification of this molecules with chromatographic steps, optional PEGylation/purification followed by loading of the necessary RNA compounds for function. Alternatively, b) the Cas9-CPP protein part is co-expressed together with the necessary RNA compounds encoded from a plasmid or integrated into a genomic locus of the host cell, extracts are prepared and the Cas9-CPP:RNA complexes are purified by chromatographic steps under conditions that maintain the Cas9-CPP:RNA complexes, optional as a final step these isolates are then pegylated and purified to homogeneity. The obtained polypeptides can be freeze dried or frozen for storage in the absence or presence of stabilizers such as glucose or trehalose.

According to a further aspect, the present invention also relates to a pharmaceutical preparation comprising a compound according to the present invention and a protein kinase inhibitor (PKI), preferably a DNA-dependent PKI, more preferred a Non Homologous End Joining (NHEJ) and V(D)J repair factor DNA-dependent PKI, especially 7-Methyl-2-[(7-methyl[1,2,4]triazolo[1,5-a]pyridin-6-yl)amino]-9-(tetrahydro-2H-pyran-4-yl)-7,9-dihydro-8H-purin-8-one (AZD7648).

Therefore, the present invention discloses the following preferred embodiments:

1. Compound comprising or consisting of a polypeptide with the general formula (I):

X₀GX₁X₂GX₃X₄X₅GX₆X₇X₈GX₉X₁₀X₁₁X₁₂X₁₃X₁₄,
wherein
G is glycine;
X₀ is present or not and, if present, is an amino acid linker;
X₁ is N or S, wherein N is asparagine and S is serine;
X₂ is S or T, wherein S is serine and T is threonine;
X₃ is present or not and, if present, is S, wherein S is serine;
X₄ is present or not and, if present, is G, wherein G is glycine;
X₅ is a basic polypeptide stretch consisting of 5 basic amino acid residues selected from R and K, wherein R is arginine and K is lysine, preferably wherein X₅ comprises at least 3 K amino acid residues, especially wherein X₅ is KKKKK or KRKKK;
X₆ is a basic amino acid residue selected from R and K, wherein R is arginine and K is lysine, preferably wherein X₆ is K;
X₇ is S or L, wherein S is serine and L is leucine;
X₈ is present or not and, if present, is a polypeptide stretch consisting of the amino acid sequence GLGS, wherein G is glycine, L is leucine and S is serine;
X₉ is a basic polypeptide stretch consisting of 3 basic amino acid residues selected from R and K, wherein R is arginine and K is lysine, preferably wherein X₉ comprises at least 2 K amino acid residues, especially wherein X₉ is KKK or KKR;
X₁₀ is present or not and, if present, is L or a polypeptide stretch consisting of the amino acid sequence DPL or DPC, wherein L is leucine, D is aspartic acid, P is proline, and C is cysteine;
X₁₁ is present or not and, if present, is a polypeptide stretch consisting of the amino acid sequence LR or GSGL, wherein L is leucine, R is arginine, G is glycine, and S is serine;
X₁₂ is present or not and, if present, is a basic polypeptide stretch with at least 20% basic amino acid residues selected from K and R and at least a P residue, preferably wherein X₁₂ is selected from KYKPKL, KYKPKLGT, or GX₃X₄X₅GX₆X₇X₈GX₉X₁₀, wherein K, R, P, L, G, T, X₃, X₄, X₅, GX₆, X₇, X₈, GX₉, and X₁₀, are defined as above and wherein Y is tyrosine;
X₁₃ is present or not and, if present, is a polypeptide stretch consisting of the amino acid sequences GS, GST, GST, GSG, GSTG, or GSGL, wherein G, S, T and L are defined as above;
X₁₄ is present or not and, if present, is an amino acid linker; wherein the polypeptide has a length from 20 to 75 amino acid residues, preferably from 24 to 65 amino acid residues, especially from 25 to 60 amino acid residues.

2. Compound according to embodiment 1, wherein X₁ is N, X₂ is S, X₃ is S, X₄ is G, X₅ is KKKKK or KRKKK, X₆ is K, X₉ is KKK or KKR, and X₁₀ is L or a polypeptide stretch consisting of the amino acid sequence DPL or DPC, wherein N, S, G, R, L, D, P and C are defined as above.

3. Compound according to embodiment 1 or 2, wherein the compound is selected from the group

GNSGSGKRKKKGKGLGKKRDPCLRKYKPKLGTGSTG(HHHHHH)
(DDM11),
GNSGKRKKKGKGSGLGSGKKRDPCLRKYKPKLGSTG(HHHHHH)
(DDM18),
GNSGSGKRKKKGKGLGKKRDPCLRKYKPKLGTGSTG(HHHHHH)
(DDM21),
GNSGSGKRKKKGKGSGLGSGKKRDPLGSTG(HHHHHH)
(DDM26),
GNSG(HHHHHH)GSTGGKRKKKGKGSGLGSGKKRDPL
(DDM27),
GNSGSGKRKKKGKGSGLGSGKKKDPLGSTG(HHHHHH)
(DDM28),
GNSG(HHHHHH)GSTGGKRKKKGKGSGLGSGKKKDPL
(DDM29),
GNSGSGKKKKKGKGSGLGSGKKRDPLGSTG(HHHHHH)
(DDM30),
GNSG(HHHHHH)GSTGGKKKKKGKGSGLGSGKKRDPL
(DDM31),
GNSGSGKKKKKGKGSGLGSGKKKDPLGSTG(HHHHHH)
(DDM32),
GNSG(HHHHHH)GSTGGKKKKKGKGSGLGSGKKKDPL
(DDM33),
GNSGSGKKKKKGKGSGLGSGKKKDPL
(DDM34),
GNSGSGKKKKKGKGSGLGSGKKKLGSTG(HHHHHH)
(DDM35),
GNSGSGKKKKKGKGSGLGSGKKKDPLGSGLGSGKKKK
KGKGSGLGSGKKKDPLGST
(DDM36),
and
GNSGSGKKKKKGKGSGLGSGKKKDPLGSGLGSGKKK
KKGKGSGLGSGKKKDPLGSTG(HHHHHH)GST
(DDM44),

wherein (HHHHHH; a histidine tag) may be present or not; preferably

GNSGSGKRKKKGKGLGKKRDPCLRKYKPKLGTGSTG
(DDM11),
GNSGKRKKKGKGSGLGSGKKRDPCLRKYKPKLGSTG
(DDM18),
GNSGSGKRKKKGKGLGKKRDPCLRKYKPKLGTGSTG
(DDM21),
GNSGSGKRKKKGKGSGLGSGKKRDPLGSTG
(DDM26),
GNSGGSTGGKRKKKGKGSGLGSGKKRDPL
(DDM27),
GNSGSGKRKKKGKGSGLGSGKKKDPLGSTG
(DDM28),
GNSGGSTGGKRKKKGKGSGLGSGKKKDPL
(DDM29),
GNSGSGKKKKKGKGSGLGSGKKRDPLGSTG
(DDM30),
GNSGGSTGGKKKKKGKGSGLGSGKKRDPL
(DDM31),
GNSGSGKKKKKGKGSGLGSGKKKDPLGSTG
(DDM32),
GNSGSGKKKKKGKGSGLGSGKKKDPL
(DDM34),
GNSGSGKKKKKGKGSGLGSGKKKDPLGSGLGSGKKK
KKGKGSGLGSGKKKDPLGST
(DDM36),
and
GNSGSGKKKKKGKGSGLGSGKKKDPLGSGLGSGKK
KKKGKGSGLGSGKKKDPLGSTGGST
(DDM44), especially
GNSGSGKKKKKGKGSGLGSGKKKDPLGSGLGSGKKK
KKGKGSGLGSGKKKDPLGST
(DDM36),
and
GNSGSGKKKKKGKGSGLGSGKKKDPLGSGLGSGK
KKKKGKGSGLGSGKKKDPLGSTGGST
(DDM44).

4. Compound according to any one of embodiments 1 to 3, wherein the compound additionally comprises a payload molecule to be delivered into a biological cell covalently attached to the polypeptide with the general formula (I), preferably a chemotherapeutic molecule, a cytotoxic molecule, a DNA damaging molecule, an anti-metabolite molecule, a therapeutic molecule, a small molecule with therapeutic effect inside a biological cell, a DNA molecule, an RNA molecule, an antibody molecule or an antibody derivative having an antibody-like function, a restriction endonuclease, a nicking enzyme, or DNA- or RNA-dependent endonucleases which show double strand breaking nuclease double strand/single strand breaking activity or no nuclease activity, more preferred wherein the payload molecule is a class II restriction endonuclease, such as PvuII, EcoRV, PvuII, HinfI, or a sc homodimer, a subunit or a functional fragment thereof; especially wherein the payload molecule is selected from the group

SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELASINIDLTKGESTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELASINIDLTKGESTHHHMNPVIIAKYRQVPWIFAIYRGIAIEAIYRLEPKD-
LEFYYDKWERKWYSDGHKDINNPKIPVKYVMEHGTKIYGSGGGSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGCCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCSGGSSH-
PDLNKLLELWPHIQEYQDLALKHGINDIFQDNGGKLLQVLLITGLTVLPGREGNDAVDNAGQE-
YELASINIDLTKGESTHHHMNPVIIAKYRQVPWIFAIYRGIAIEAI-
YRLEPKDLEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
and
SHPDLNKLLELWPHIQEYQDLALKHGINDIFQDNGGKLLQVLLITGLTVLPGREG-
NDAVDNAGQEYELASINIDLTKGESTHHHMNPVIIAKYRQVPWIFAIYRGIAIEAIYRLEPKD-
LEFYYDKWERKWYSDGHKDINNPKIPVKYVMEHGTKIYGSGGCCCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIY.

5. Compound according to any one of embodiments 1 to 4, wherein the compound additionally comprises a homopolymer moiety covalently attached to the polypeptide with the general formula (I), preferably wherein the homopolymer is selected from the group of polyethylene glycol (PEG), especially a PEG with a MW of 5 to 30 kDa; dextran, polysialic acids, hyaluronic acid, dextrin, hydroxyethyl starch, or poly(2-ethyl 2-oxazoline.

6. Compound according to any one of embodiments 1 to 5, wherein the compound additionally comprises a restriction endonuclease as a payload molecule to be delivered into a biological cell covalently attached to the polypeptide with the general formula (I), preferably a class II restriction endonuclease, especially a PvuII restriction endonuclease or a derivative thereof wherein the derivative is a single chain PvuII restriction endonuclease, such as the derivative with the amino acid sequence

SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGG,
SGGSSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGNSG,
MSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGC,
CSGGSSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
MSHPDLNKLLELWPHIQEYQDLALKHGINDIFQDNGGKLLQVLLITGLTVLPGREG-
NDAVDNAGQEYELKSINIDLTKGESTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
MSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIY,
MSHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGSGGS,
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGSGGS,
or
GSGGSGGSSHPDLNKLLELWPHIQEYQDLALKHGIN-
DIFQDNGGKLLQVLLITGLTVLPGREGNDAVDNAGQEYELKSINIDLTKGESTHHHMNPVIIA-
KYRQVPWIFAIYRGIAIEAIYRLEPKDLEFYYDKWERKWYSDGHKDINNPKIPVKYVME-
HGTKIY.

7. Compound according to any one of embodiments 1 to 6, wherein the polypeptide with the general formula (I) is covalently linked to another moiety by a linker, wherein the another moiety is preferably a payload molecule to be delivered into a biological cell, a labelling group, and/or a homopolymer.

8. Compound according to any one of embodiments 1 to 7, wherein the compound further comprises a capping group, preferably an alkoxy, especially a methoxy, ethoxy, propoxy, or butoxy group; a halogen atom; or a tosylate; isocyanate, hydrazine hydrate, maleimide, orthopyridyl disulfide, N-succinimidyloxy, sulfo-N-succinimidyloxy, 1-benzotriazol, 1-imidazolyloxy, p-nitrophenyloxy, or aldehyde.

9. Compound according to any one of embodiments 1 to 8, wherein the compound further comprises a small molecule as payload, wherein the small molecule is a medicinal organic compound having a molecular mass of less than 1000 Da, preferably of 300 to 1000 Da, especially of 300 to 700 Da.

10. Compound according to any one of embodiments 1 to 9, wherein the compound further comprises secondary metabolites as payload, including alkaloids, terpenoids, steroids, glycosides, natural phenols, phenazines, polyketides, fatty acid synthase products, nonribosomal peptides, macrolactones, and polyphenols.

11. Compound according to any one of embodiments 1 to 10, wherein the compound further comprises as payload cytotoxic agents including, for example, natural or synthetic tubulysins; natural or synthetic epothilones; duocarmycines; auristatins; maytansinoids; calicheamycin; mesothelins; anthracyclins; dihydrofolate reductase inhibitors; and thymidylate synthase inhibitors; DNA intercalators; DNA cleavers; topoisomerase inhibitors; the vinca drugs; the mitomycins; the bleomycins; the cytotoxic nucleosides; the pteridine family of drugs; diynenes; the podophyllotoxins; differentiation inducers; and taxanes; erlotinib (TARCEVAR®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®), Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), and doxorubicin (ADRIAMYCIN®), Akti-1/2, HPPD, and rapamycin. Also useful cytotoxic agents include: oxaliplatin (ELOXATIN®, Sanofi), bortezomib (VELCADE®, Millennium Pharm.), sutent (SUNITINIB®, SU1 1248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1 126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), vandetanib (rINN, ZD6474, ZACTIMA®, AstraZeneca), chlorambucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, calicheamicin gamma 1, calicheamicin omega 11 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and known functional derivatives thereof;
anti-hormonal agents that act to regulate or inhibit hormone action on tumors such as anti-estrogens and selective estrogen receptor modulators (SERMs), including, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); aromatase inhibitors that inhibit the enzyme aromatase, which regulates estrogen production in the adrenal glands, such as, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); anti-androgens such as flutamide, nilutamide, bicalutamide, leuprolide, and goserelin; as well as troxacitabine (a 1,3-dioxolane nucleoside cytosine analog); protein kinase inhibitors such as MEK inhibitors; lipid kinase inhibitors; antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); ribozymes such as VEGF expression inhibitors (e.g., ANGIOZYME®) and HER2 expression inhibitors; vaccines such as gene therapy vaccines, m-RNA vaccines, ALLOVECTIN®, LEUVECTIN®, and VAXID®; PROLEUKIN® rIL-2; topoisomerase 1 inhibitors such as LURTOTECAN®; ABARELIX® rmRH; anti-angiogenic agents such as bevacizumab (AVASTIN®, Genentech); leurosine, carminomycin, tallysomycin, podophyllotoxin, retinoic acid, butyric acid, N⁸-acetyl spermidine, and pharmaceutically acceptable salts, acids and any known functional analogues thereof.

12. Compound according to any one of embodiments 1 to 11, wherein the compound further comprises as payload therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG™, 2C4, Genentech), trastuzumab (HERCEPTIN®, Genentech), tositumomab (Bexxar, Corixia), and the humanized monoclonal antibodies alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

13. Compound according to any one of embodiments 1 to 12, wherein the compound further comprises as payload a restriction endonuclease, preferably a type II restriction endonuclease, especially a type II restriction endonucleases selected from AatII, AbaSI, Acc65I, AccI, AciI, AclI, AcuI, AfeI, AflII, AflIII, AgeI, AhdI, AleI, AluI, AlwI, AlwNI, ApaI, ApaLI, ApeKI, ApoI, AscI, AseI, AsiSI, AvaI, AvaII, BaeGI, BaeI, BamHI, BanI, BanII, BbsI, BbvCI, BbvI, BccI, BceAI, BcgI, BciVI, BclI, BcoDI, BfaI, BfuAI, BfuCI, BglI, BglII, BlpI, BmgBI, BmrI, BmtI, BpmI, BpulOI, BpuEI, BsaAI, BsaBI, BsaHI, BsaI, BsaJI, BsaWI, BsaXI, BseRI, BseYI, BsgI, BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFI, BsrGI, BsrI, BssHII, BssKI, BssSaI, BstAPI, BstBI, BstEII, Bst, BstUI, BstXI, BstYI, BstZ17I, Bsu36I, BtgI, BtgZI, BtsaI, BtsCI, Cac8I, ClaI, CspCI, CviAII, CviKI-1, CviQI, DdeI, DpnI, DpnII, DraI, DraIII, DrdI, EaeI, EagI, EarI, EciI, Eco53kI, EcoNI, EcoO109I, EcoP15I, EcoRI, EcoRV, FatI, FauI, Fnu4HI, FokI, FseI, FspEI, FspI, HaeIII, HgaI, HhaI, HincII, HindIII, HinfI, HinPlI, HpaI, HpaII, HphI, Hpyl66II, Hpyl88I, Hpyl88III, Hpy99I, HpyAV, HpyCH4III, HpyCH4IV, HpyCH4V, I-CeuI, I-SceI, KasI, KpnI, LpnPI, MboI, MboII, MfeI, MluCI, MluI, MlyI, MmeI, MnlI, MscI, MseI, MslI, MspA1I, MspI, MspJI, MwoI, NaeI, NarI, Nb.BbvCI, Nb.BsmI, Nb.BsrDI, Nb.BtsI, NciI, NcoI, NdeI, NgoMIV, NheI, NlaIII, NlaIV, NmeAIII, NotI, NruI, NsiI, NspI, Nt.AlwI, Nt.BbvCI, Nt.BsmAI, Nt.BspQI, Nt.BstNBI, Nt.CviPII, PacI, PaeR7I, PciI, PflFI, PflMI, PI-PspI, PI-SceI, PleI, PluTI, PmeI, PmlI, PpuMI, PshAI, PsiI, PspGI, PspOMI, PspXI, PstI, PvuI, PvuII, RsaI, RsrII, Sac, SacII, SalI, SapI, Sau3AI, Sau96I, SbfI, ScaI, ScaI, ScrFI, SexAI, SfaNI, SfcI, SfiI, SfoI, SgrAI, SmaI, SmlI, SnaBI, SpeI, SphI, SspI, StuI, StyD4I, StyI, SwaI, TaqaI, TfiI, TseI, Tsp45I, TspMI, TspRI, Tth111I, XbaI, XcmI, XhoI, XhoI, XmaI, XmnI, ZraI

14. Compound according to any one of embodiments 1 to 13, wherein the compound further comprises as payload a nicking enzyme, preferably selected from nicking versions of AatII, Acc65I, AccI, AciI, AclI, AcuI, AflII, AgeI, AhdI, AluI, AlwNI, ApaI, ApaLI, ApeKI, ApoI, AscI, AseI, AvaI, AvaII, AvrII, BaeGI, BaeI, BamHI, BbsI, BbvI, BciVI, BclI, BcoDI, BfuAI, BfuCI, BglI, BglII, BlpI, BmgBI, BpmI, BpuEI, BsaAI, BsaHI, BsaWI, BsaXI, BseRI, BsgI, BsiEI, BsiWI, BslI, BsmAI, BsmBI, BsmI, BsoBI, Bsp1286I, BspCNI, BspEI, BspHI, BspQI, BsrBI, BsrDI, BsrGI, BsrI, BssHII, BssKI, BstBI, BstEII, BstNI, BstUI, BstXI, BstYI, BstZ17I, Bsu36I, BtgI, BtsCI, Cac8I, ClaI, CspCI, CviAII, CviQI, DdeI, DpnI, DpnII, DraI, DraIII-HF®, DrdI, EagI, EarI, Eco53kI, EcoNI, EcoO109I, EcoP15I, EcoRI, EcoRV, Fnu4HI, FseI, FspEI, FspI, HaeIII, HhaI, HincII, HinfI, HinPlI, HpaII, HphI, Hpy166II, HpyAV, HpyCH4IV, HpyCH4V, I-CeuI, I-SceI, KpnI, LpnPI, MboI, McrBC, MluCI, MluI, MlyI, MmeI, MnlI, MseI, MslI, MspA1I, MspI, MspJI, MwoI, Nb.BbvCI, Nb.BsmI, Nb.BsrDI, Nb.BtsI, NciI, NcoI, NdeI, NgoMIV, NheI, NlaIII, NotI, NruI, NsiI, NspI, Nt.AlwI, Nt.BbvCI, Nt.BsmAI, Nt.BspQI, Nt.BstNBI, Nt.CviPII, PacI, PaeR7I, PflFI, PflMI, PI-PspI, PI-SceI, PmeI, PmlI, PpuMI, PshAI, PstI, PvuI, PvuII, RsaI, SacI, SacII, SalI, SapI, SbfI, SfiI, SfoI, SmaI, SpeI, StuI, StyD4I, StyI, SwaI, TaqaI, TfiI, TseI, TspMI, TspRI, Tth111I, XbaI, XhoI, XmaI, and XmnI.

15. Compound according to any one of embodiments 1 to 14, wherein the compound further comprises as payload zinc finger nucleases (ZFNs), zinc finger transcription activators (ZFAs), zinc finger transcription repressors (ZFRs) and zinc finger methylases (ZFMs); enzymes in disease for enzyme-replacement therapy, such as glucose-6-phosphate dehydrogenase (EC 1.1.1.49) (G6PD), 21-hydroxylase (EC 1.14.99.10), steroid 11β-monooxygenase (EC 1.14.15.4), 3α-hydroxysteroid-3-dehydrogenase (EC 1.1.1.50), Steroid 17amonooxygenase(EC 1.14.99.9), cholesterol monooxygenase (EC1.14.15.6)), Glucosylceramidase (EC 3.2.1.45), galactosylceramidase (EC 3.2.1.46), Pyruvate kinase (EC 2.7.1.40), α-galactosidase(EC3.2.1.22), β-galactosidase (EC3.2.1.23)acid α-glucosidase (GAA), α-Amylase (EC 3.2.1.1), β-amylase (EC 3.2.1.2.), collagenase (EC 3.4.24.7), glutaminase(EC 3.5.1.2), asparaginase (EC 3.5.1.1), lysozyme (EC 3.2.1.17), bilirubin oxydase (EC 1.3.3.5), Thiosulfate sulfur-transferase (EC 2.8.1.1), β-lactamase (EC 3.5.2.6),streptokinase, urokinase (EC 3.4.21.73), hyalurono-glucosaminidase (EC 3.2.1.35), hyalurono-glucuronidase (EC 3.2.1.36), hyaluronate lyase (EC 4.2.2.1), Superoxide dismutase (EC 1.15.1.1), urease (EC 3.5.1.5), arginase (EC 3.5.3.1), glutamyl-transferase (EC 2.3.2.2.), glutathione-S-transferase (EC 6.3.1.13), creatine kinase (EC 2.7.3.2), aldolase (EC 4.1.2.13), lipase (EC 3.1.1.79), hydroxymethylglutaryl-CoA reductase (EC 1.1.1.34), Xantin oxydase, PRPP Synthase (EC 2.7.6.1), hypoxantine-guanine-Phosphoribosyltransferase (EC 2.4.2.8), DNAse (EC 3.1.22.1), RNAse (EC 3.1.27.5), Branched-chain ketoacid dehydrogenase (EC 1.2.4.4), Adenosine deaminase (EC 3.5.4.4), hexoseaminidase (EC 3.2.1.52), β-galactosidase (EC 3.2.1.23), sphingomyelinase(EC 3.1.4.12), ceramidase (EC 3.5.1.23), homogenitisate-oxydase, β-glucosidase (EC 3.2.1.21), lactase (EC 3.2.1.108), HIV-protease (EC 3.4.23.16), dihydro-folate reductase (EC 1.5.1.3), alcohol-dehydrogenase (EC 1.1.1.2), Iduronate-2-sulfatase (EC 3.1.6.13), or sphingomyelin phosphodiesterase (EC 3.1.4.12).

16. Compound according to any one of embodiments 1 to 15, wherein the compound further comprises as payload two monomeric subunits or parts thereof of a type II restriction endonuclease, preferably, two monomeric alpha-helical subunits of a type II restriction endonuclease covalently connected by a linker to the polypeptide with the general formula (I).

17. Compound according to any one of embodiments 1 to 16, wherein the compound further comprises as payload two monomeric alpha-helical subunits of PvuII, preferably wherein the two monomeric subunits or parts thereof of the type II restriction endonuclease PvuII are covalently connected by a linker to the polypeptide with the general formula (I).

18. Compound according to any one of embodiments 1 to 17, wherein the compound further comprises a linker, wherein the linker is an amino acid linker of 1 to 20 amino acid residues in length, preferably from 1 to 15 amino acid residues, more preferred from 7 to 13 amino acid residues, especially of 8 to 12 amino acid residues.

19. Compound according to any one of embodiment 1 to 18, wherein the compound further comprises a linker, wherein the linker is an amino acid linker, selected from a linker comprising or consisting of cysteine, serine or glycine residue, preferably a single or two adjacent cysteine residue (s); an amino acid sequence comprising glycine and serine residues or comprising or consisting of the amino acid sequences GSG, SGG, GGC, GCS, CSG, GGS, GSGG, SGGS, GSGGC, CSGGS, GSGGSGGS, GSGGCCSGGS, GSGGCCCSGGS, or GSGGCSGGS.

20. Compound according to any one of embodiments 1 to 19, wherein the compound further comprises as payload an editing nuclease, a CRISPR nuclease and/or hybrids thereof.

21. Compound according to any one of embodiments 1 to 20, wherein the general formula (I) contains a single arginine and/or a single cysteine or not more than two arginine residues and/or not more than two cysteine residues

22. Compound according to any one of embodiments 1 to 21, wherein the general formula (I) lacks arginine and/or cysteine, preferably wherein the general formula (I) lacks arginine and cysteine.

23. Compound according to any one of embodiments 1 to 22, comprising at least two polypeptides with the general formula (I).

24. Compound according to any one of embodiments 1 to 23, wherein the compound is selected from the group GNSGSGKKKKKGKGSGLGSGKKKDPL (DDM34) and

GNSGSGKKKKKGKGSGLGSGKKKDPLGSGLGSGKKKKK
GKGSGLGSGKKKDPLGST
(DDM36).

25. Compound according to any one of embodiments 1 to 24, wherein the polypeptide with the general formula (I) is covalently coupled to the C-terminus of a payload molecule to be delivered into a biological cell, preferably a compound selected from the group

SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKI-
YGNSGSGKKKKKGKGSGLGSGKKKDPLGSGLGSGKKKKKGKGSGLGSGKKKDPLGST,
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKI-
YGNSGSGKKKKKGKGSGLGSGKKKDPLGSGLGSGKKKKKGKGSGLGSGKKKDPLGST,
SHPDLNKLLELWPHIQEYQDLALKHGINDIFQDNGGKLLQVLLITGLTVLPGREG-
NDAVDNAGQEYELKSINIDLIKGESTHHHMNPVIIAKYRQVPWIFAIYRGIAIEAIYRLEPKD-
LEFYYDKWERKWYSDGHKDINNPKIPVKYVMEHGTKIYGSGGGSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELKSINIDLTKGESTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREG-
NDAVDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSH-
PDLNKLLELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST,
and
SHPDLNKLLELWPHIQEYQDLALKHGINDIFQDNGGKLLQVLLITGLTVLPGREG-
NDAVDNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSH-
PDLNKLLELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAI-
YRLEPKDLEFYYDKWERKWYSDGHKDINNPKIPVKYVMEHGTKIYGNSGSGKKKKKGKGSGLG-
SGKKKDPLGSGLGSGKKKKKGKGSGLGSGKKKDPLGST.

26. Compound according to any one of embodiments 1 to 25, wherein the polypeptide with the general formula (I) is covalently coupled to the C-terminus of a payload molecule to be delivered into a biological cell which is a class II restriction endonuclease, preferably PvuII or a subunit thereof, wherein the payload molecule is optionally attached at its C-terminus to a linker molecule comprising 8 to 12 amino acids comprising or consisting of glycine, serine and/or cysteine residues, and wherein the polypeptide with the general formula (I) is covalently coupled to the C-terminus of the payload molecule or the linker molecule, wherein the compound comprises one or two payload molecule(s), optionally separated by the linker molecule comprising 8 to 12 amino acids comprising or consisting of glycine, serine and/or cysteine residues.

27. Compound according to any one of embodiments 1 to 26, wherein the polypeptide with the general formula (I) is covalently coupled to the C-terminus of a payload molecule to be delivered into a biological cell, wherein the compound comprises a first payload molecule which is the first subunit of PvuII linked at its C-terminus to a linker molecule comprising 8 to 12 amino acids comprising or consisting of glycine, serine and/or cysteine residues, wherein the linker molecule is attached at its C-terminus to the second subunit of PvuII and wherein the polypeptide with the general formula (I) is covalently coupled to the C-terminus of the second subunit of PvuII.

28. Compound according to any one of embodiments 1 to 27, for use in the treatment of a tumour patient, preferably wherein the tumour patient is suffering from neuroblastoma, colon carcinoma, hepatocellular carcinoma, Small Cell Lung carcinoma, ovarian carcinoma, lung carcinoma, bladder carcinoma, prostate carcinoma, uterus carcinoma, cervix carcinoma, placental carcinoma, breast carcinoma, kidney carcinoma, liver carcinoma, pancreas carcinoma, muscle carcinoma, skin carcinoma, connective tissue carcinoma, bone carcinoma, and any of these carcinomas wherein the patient has already developed metastases, especially for the treatment of a patient having neuroblastoma, colon carcinoma, hepatocellular (liver) carcinoma, Small Cell Lung carcinoma, lung carcinoma, breast carcinoma, pancreas carcinoma, and any of these carcinomas wherein the patient has already developed metastases.

29. Method of treatment of a tumour patient wherein an effective amount of the compound according to any one of embodiments 1 to 27, is administered to a patient in need thereof, preferably wherein the tumour patient is suffering from neuroblastoma, colon carcinoma, hepatocellular carcinoma, Small Cell Lung carcinoma, ovarian carcinoma, lung carcinoma, bladder carcinoma, prostate carcinoma, uterus carcinoma, cervix carcinoma, placental carcinoma, breast carcinoma, kidney carcinoma, liver carcinoma, pancreas carcinoma, muscle carcinoma, skin carcinoma, connective tissue carcinoma, bone carcinoma, and any of these carcinomas wherein the patient has already developed metastases, especially for the treatment of a patient having neuroblastoma, colon carcinoma, hepatocellular (liver) carcinoma, Small Cell Lung carcinoma, lung carcinoma, breast carcinoma, pancreas carcinoma, and any of these carcinomas wherein the patient has already developed metastases.

30. Use of a compound according to any one of embodiments 1 to 27 for the manufacture of a medicament, preferably for the treatment of a tumour patient, more preferred for the treatment of a patient having neuroblastoma, colon carcinoma, hepatocellular carcinoma, Small Cell Lung carcinoma, ovarian carcinoma, lung carcinoma, bladder carcinoma, prostate carcinoma, uterus carcinoma, cervix carcinoma, placental carcinoma, breast carcinoma, kidney carcinoma, liver carcinoma, pancreas carcinoma, muscle carcinoma, skin carcinoma, connective tissue carcinoma, bone carcinoma, and any of these carcinomas wherein the patient has already developed metastases, especially for the treatment of a patient having neuroblastoma, colon carcinoma, hepatocellular (liver) carcinoma, Small Cell Lung carcinoma, lung carcinoma, breast carcinoma, pancreas carcinoma, and any of these carcinomas wherein the patient has already developed metastases.

31. Pharmaceutical preparation comprising a compound according to any one of embodiments 1 to 27 and a protein kinase inhibitor (PKI), preferably a DNA-dependent PKI, more preferred a Non Homologous End Joining (NHEJ) and V(D)J repair factor DNA-dependent PKI, especially 7-Methyl-2-[(7-methyl[1,2,4]triazolo[1,5-a]pyridin-6-yl)amino]-9-(tetrahydro-2H-pyran-4-yl)-7,9-dihydro-8H-purin-8-one (AZD7648; Fok et al., Nat. Comm. 10 (2019), 5056).

The present invention is further illustrated by way of the following examples and the figures, yet without to be limited thereto.

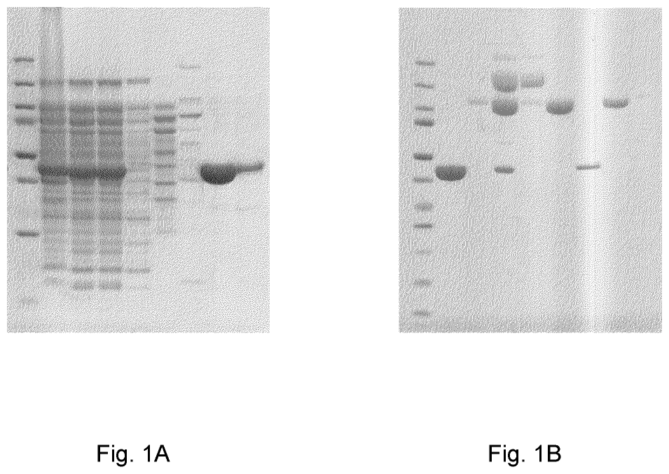

FIG. 1A: SDS gel profile of CROMOC36 purification is a three-column downstream process for drug intermediate. SDS gel profile of CROMOC36 purification. Lanes from left to right: Molecular mass markers, total extract, peak fraction Affinity chromatography, anion chromatography flow through, flow through cation chromatography, low salt elution cation chromatography, medium salt elution cation chromatography, first peak fraction (I) “preCROMOC”, high salt elution cation chromatography, peak fraction (II) high salt elution cation chromatography. The gels show a highly enriched protein of the invention can be reached with the described process.

FIG. 1B: SDS gel profile of CROMOC36 from the PEGylation of the peak fraction in FIG. 1A. and subsequent purification final product: right lane. From left to right: Lane 1, Molecular mass markers, Lane 2) peak fraction(I) from cation chromatography, Lanes 3-7 PEG reaction intermediates. Lane 8 final product of purified PEGylated molecule “CROMOC36” used in the Examples. The gels shows that a highly enriched protein of the invention can be reached with the described process.

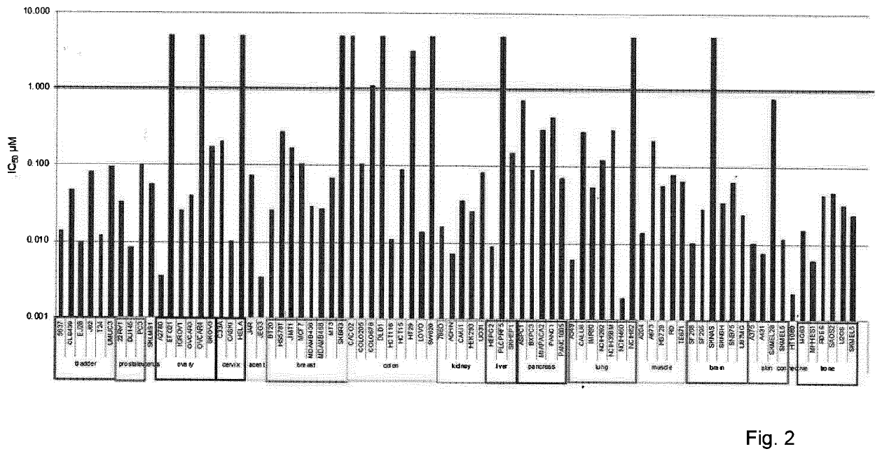

FIG. 2: CROMOC36 shows anti-proliferative activity at nM concentrations in tumor cells representative for different tumor indications (55/80 cell lines IC₅₀≤100 nM).

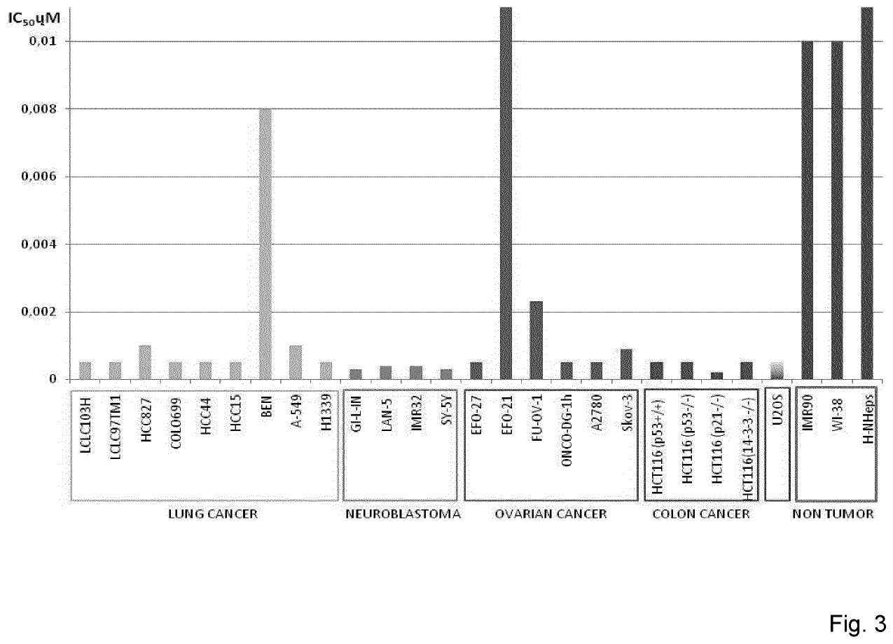

FIG. 3: CROMOC36 shows antiproliferative activity at nM concentrations and is 40->100 times less cytotoxic to immortalized non-tumor cells than to cancer cells.

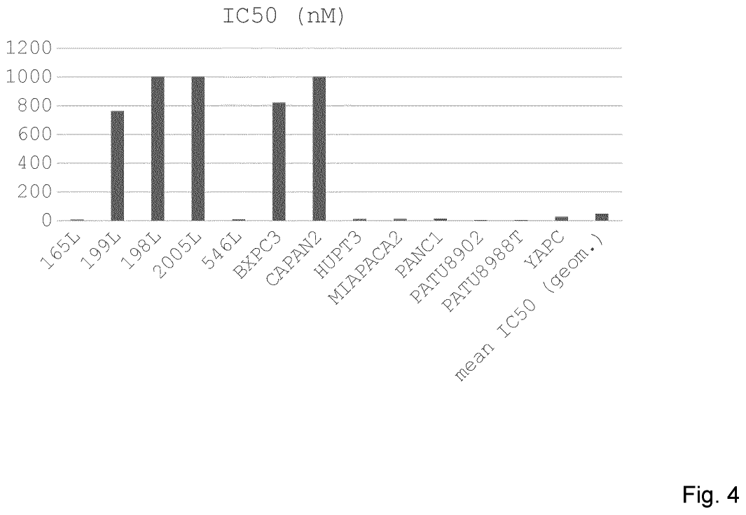

FIG. 4: CROMOC36—in vitro activity Human pancreatic carcinoma cell lines. IC50 were assessed by the propidium iodide monolayer assay for 96 hours. CROMOC36 showed selective and concentration-dependent antitumor activity towards the human pancreatic cell lines tested. CROMOC36 was most sensitive on PATU8902, PATU8988T, PAXF 1657L and PAXF 546L cell lines. The result shows resistant cells with IC50 in the low μM range and highly sensitive, 1000 times more sensitive cell lines in the low nM range.

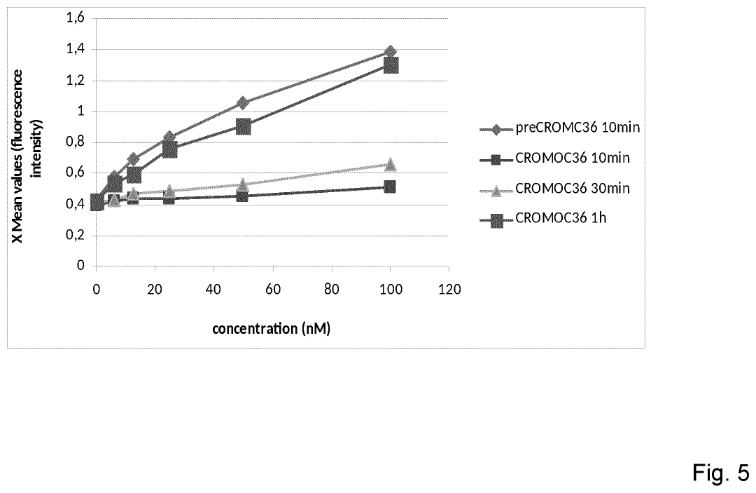

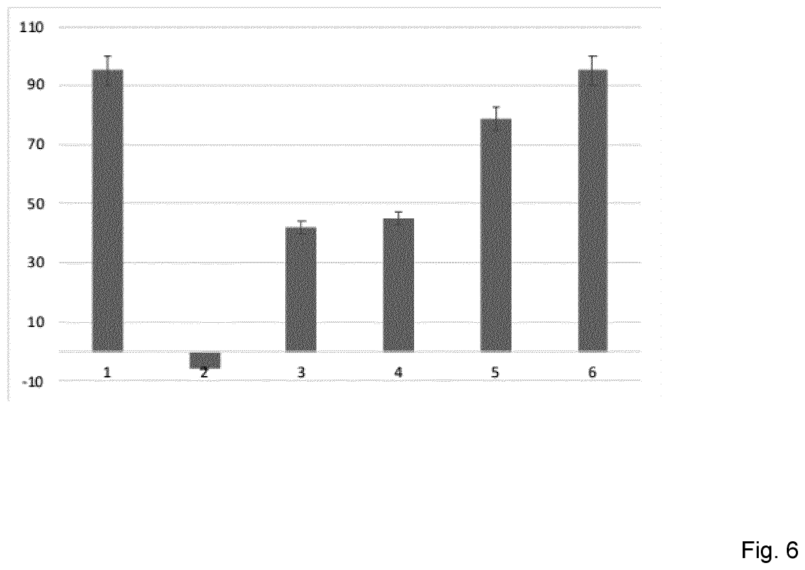

FIG. 5: Comparison of the uptake kinetics of PEGylated CROMOC36-AF680 (CROMOC36-AF680) versus non-PEGylated preCROMOC-AF680 is shown. Fluorescence intensities were measured by Flow cytometry. U2OS cells were incubated for the indicated time points and cells were fixed after intensive washing of the surface bound molecules with ice cold PBS.

FIG. 6: CROMOC endocytosis inhibitor study to evaluate the pathways important for membrane crossing of CROMOC. These studies show that uptake of CROMOC36 is ATP dependent and is via clathrin as well as caveolin dependent endocytosis for DDM targeted transport and no micropinocytosis takes place. Conditions tested with CROMOC36 were uptake at 4° C. (1) or in the presence of Cytocholasine D (2, CD); Chloropromazine (3, CPZ); Methyl-ßCyclodextrin (4, MB CD); Chloropromazine and Methyl-β-Cyclodextrin (5, CPZ+MB3CD); Heparin (5, HP) and cellular uptake was measured using flow cytometry.

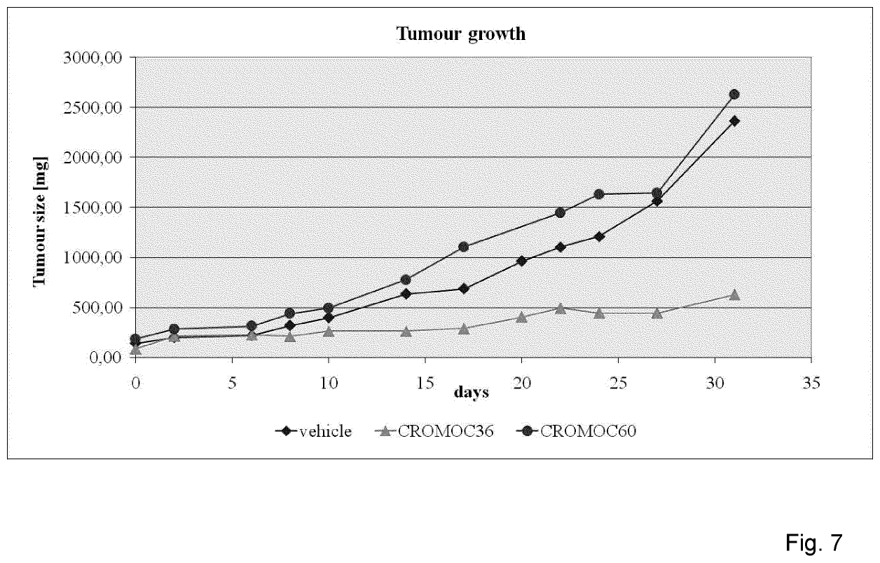

FIG. 7: HCT116 p53−/− human colon carcinoma xenograft. Treatment on advanced tumors (volume >50 mm³). Dose: CROMOC36 4 mg/kg; CROMOC60 4 mg/kg. Treatment: s.c. administration on day 0, 1, 2, 3, 6, 7, 8, 9, 10, 20, 22, 24.

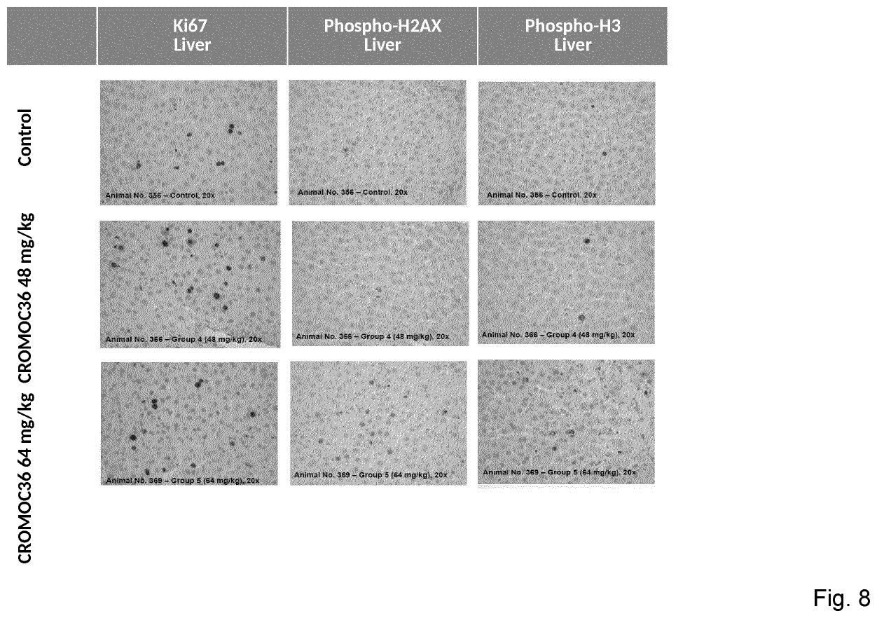

FIG. 8: Immunohistochemistry (IHC) analysis paraffin embedded liver specimens of Male Sprague Dawley Rats after single IV treatment with CROMOC36. Samples were collected after 13-15 days and IHC was used to assess the Histone surrogate-markers for DNA double strand breaks Phospho-H2AX and Phospho-H3 for analyzing the persistence of DNA double stand breaks (DSBs) at very high CROMOC concentrations in tumors in comparison to the KI67marker. This proofs that DSBs are induced by the CROMOC molecules and that the DDM sequences can guide payloads into the nucleus of a cell in vivo.

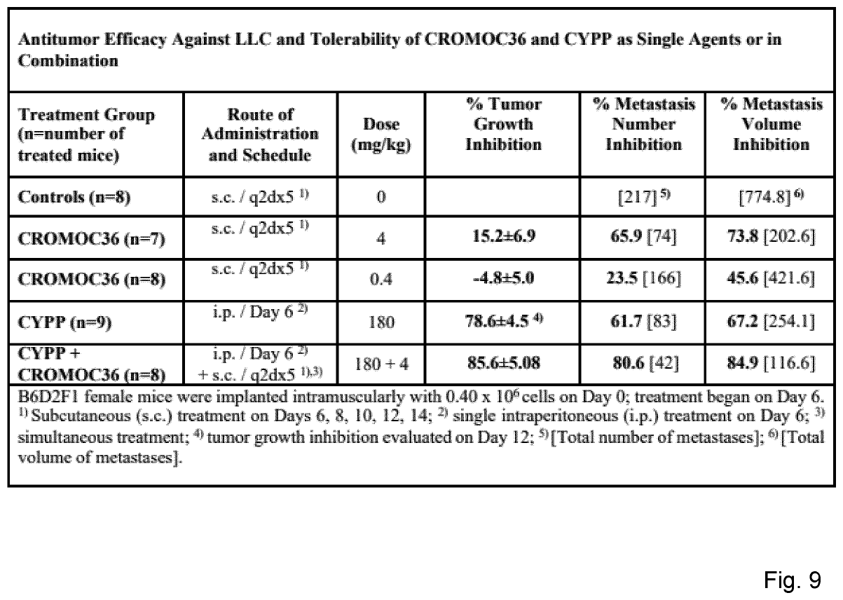

FIG. 9: In Vivo Antitumor and Antimetastatic Activity of CROMOC36 in Combination with Cyclophosphamide on Murine Lewis Lung Carcinoma.

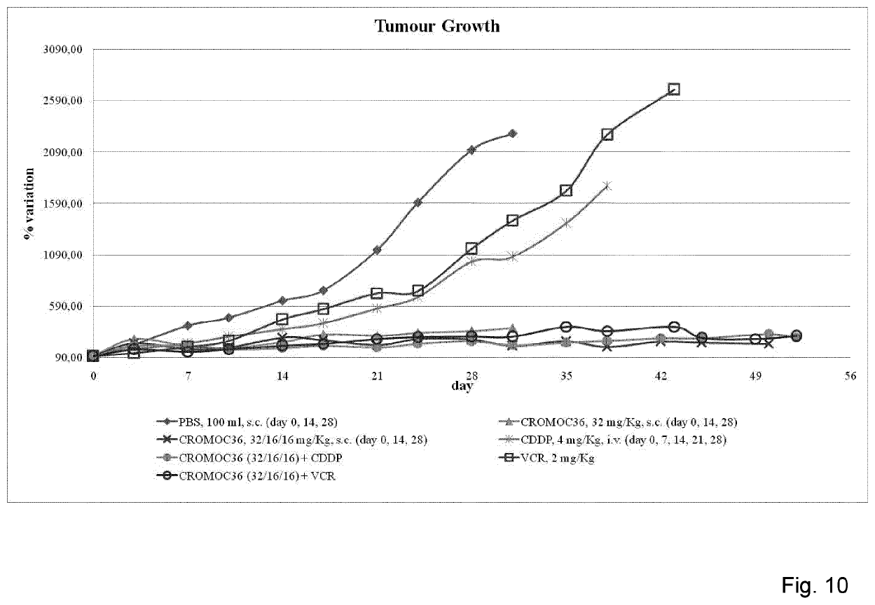

FIG. 10: CROMOC36 Antitumor Efficacy on HCT116 (p53−/−) Human Colon Carcinoma Xenograft Model as a Single Agent and in Combination Therapy with Cisplatin or Vincristine.

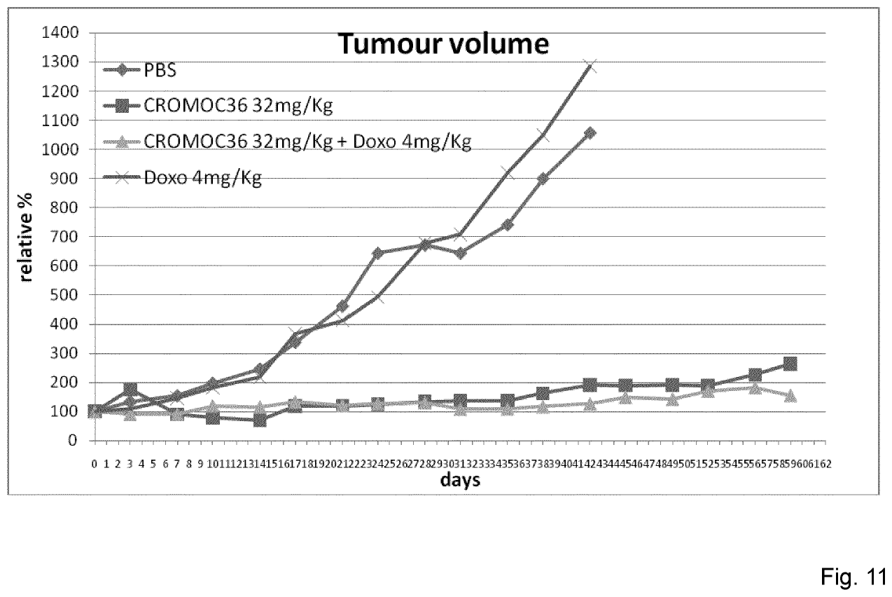

FIG. 11: IMR32 s.c. implanted. Treatment on xenograft mice with advanced tumors (volume >50 mm³). Variation in tumor volume expressed as percentage with respect to initial volume (day 0; designated as 100%). Final measurements for vehicle (PBS) and Doxorubicin groups were taken on day 42. For the remaining groups, final measurements were taken on day 63, the termination day of the experiment. Growth reduction compared to controls on day 42 (p<0.01).

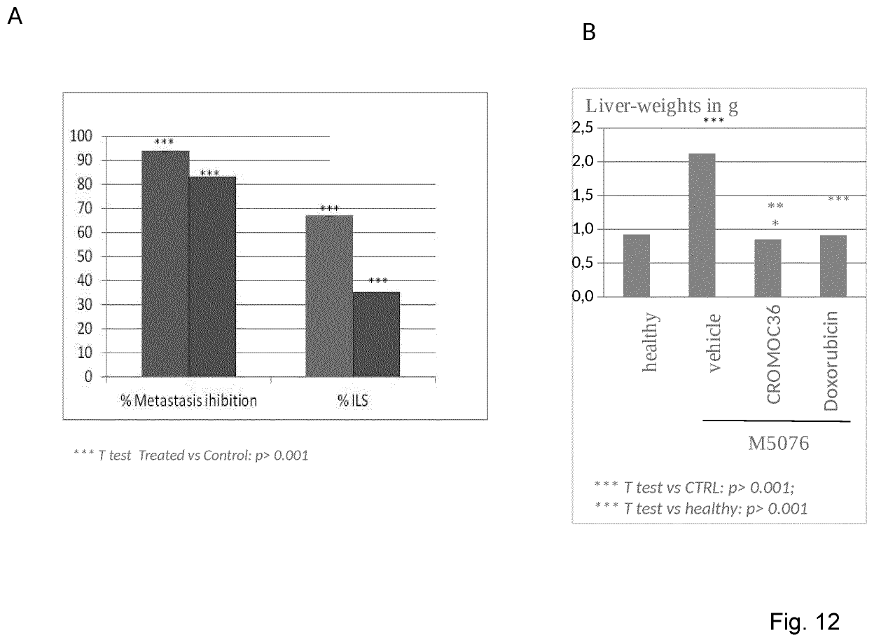

FIG. 12: CROMOC36 has strong antimetastatic activity in Liver M5076 allograft, i.v. Route (M5076). 12A.) Blue CROMOC 16 mg/kg (q7dx3), red Doxorubicin 6 mg/kg (q4dx3). Left % metastasis inhibition, right ILS %. 12B.) Liver weights.

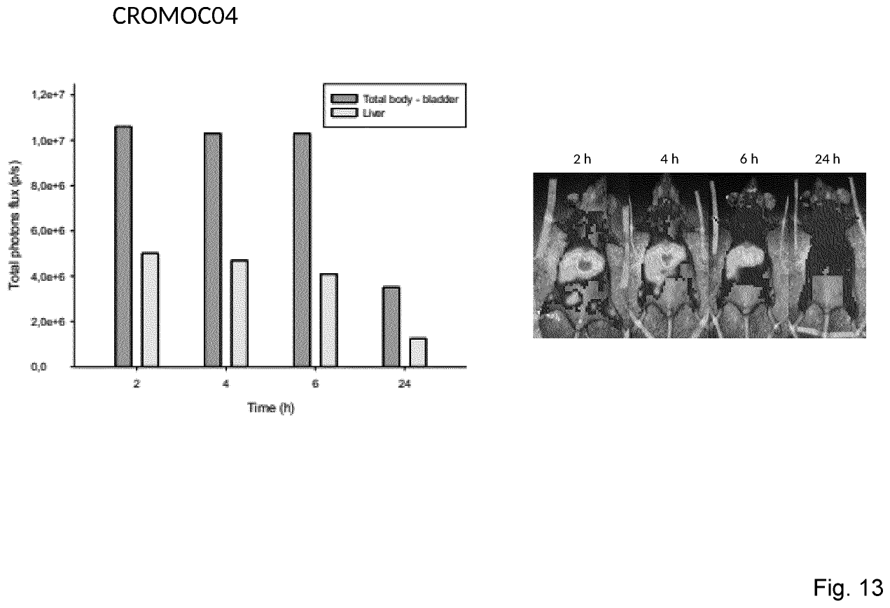

FIG. 13: In vivo image—bio-distribution CROMOC04.

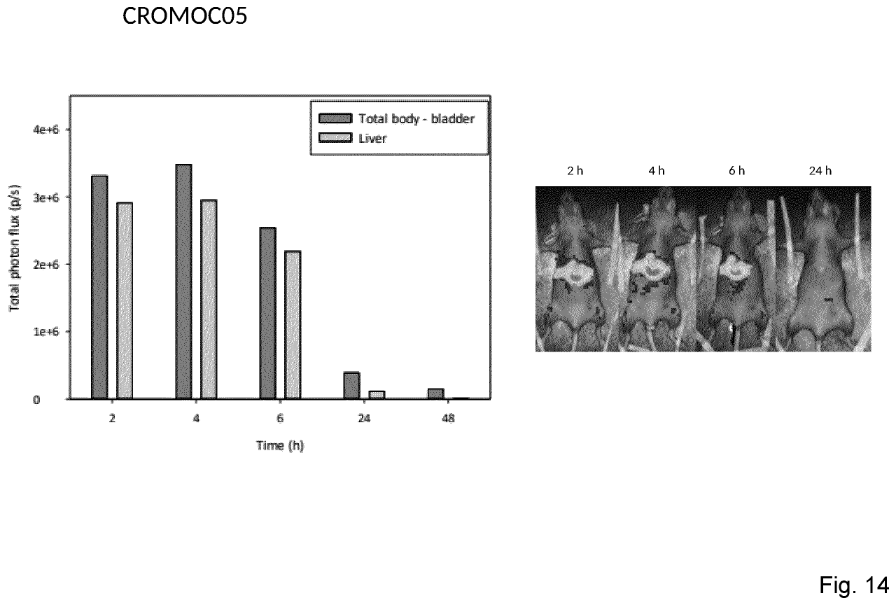

FIG. 14: In vivo image—bio-distribution CROMOC05.

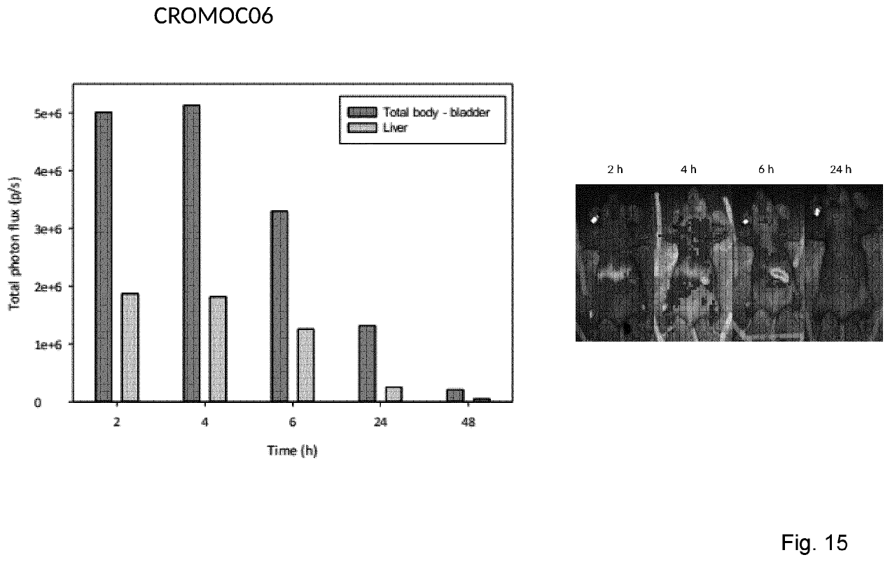

FIG. 15: In vivo image—bio-distribution CROMOC06.

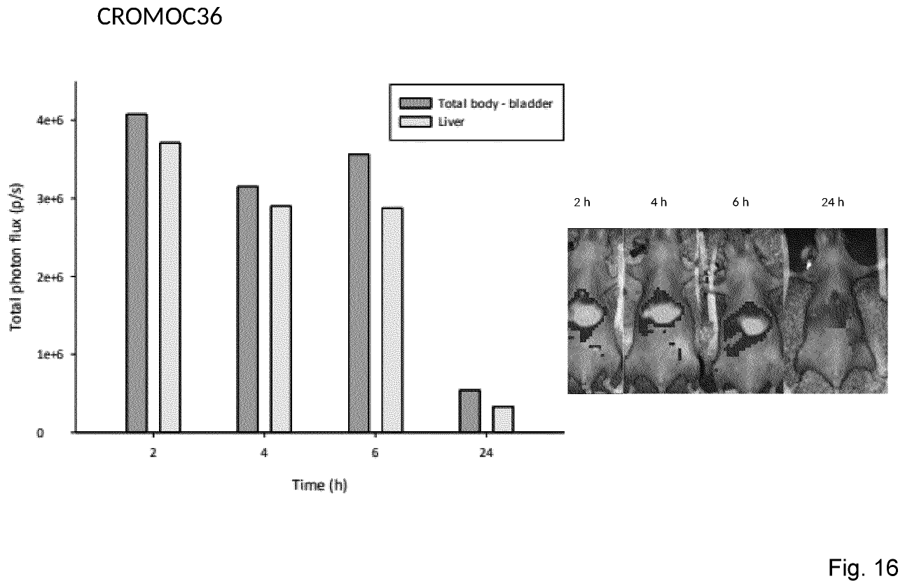

FIG. 16: In vivo image—bio-distribution CROMOC36.

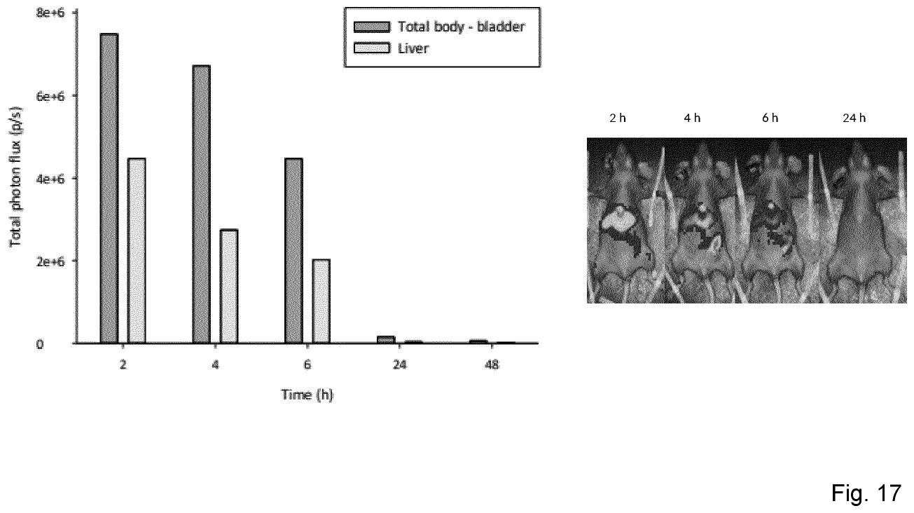

FIG. 17: In vivo image—bio-distribution proCROMOC36.

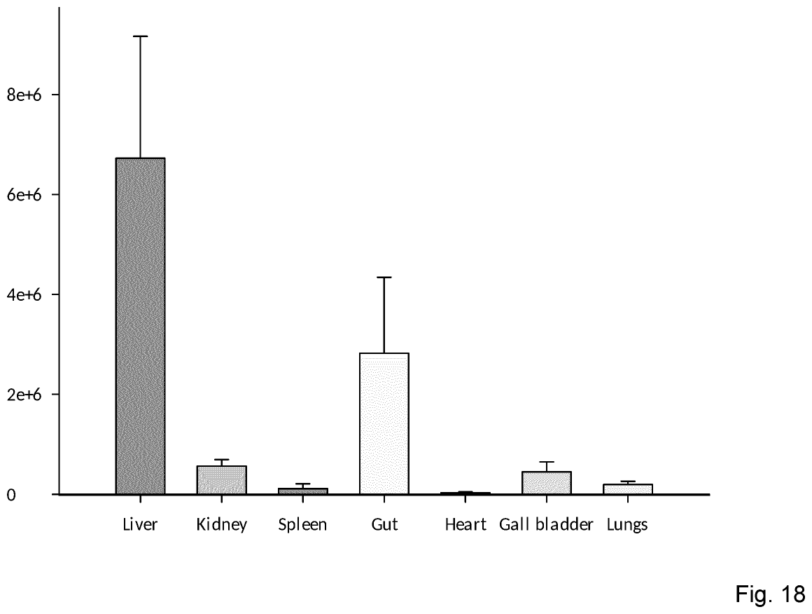

FIG. 18: In vivo organ-distribution in healthy mice of CROMOC36 molecules.

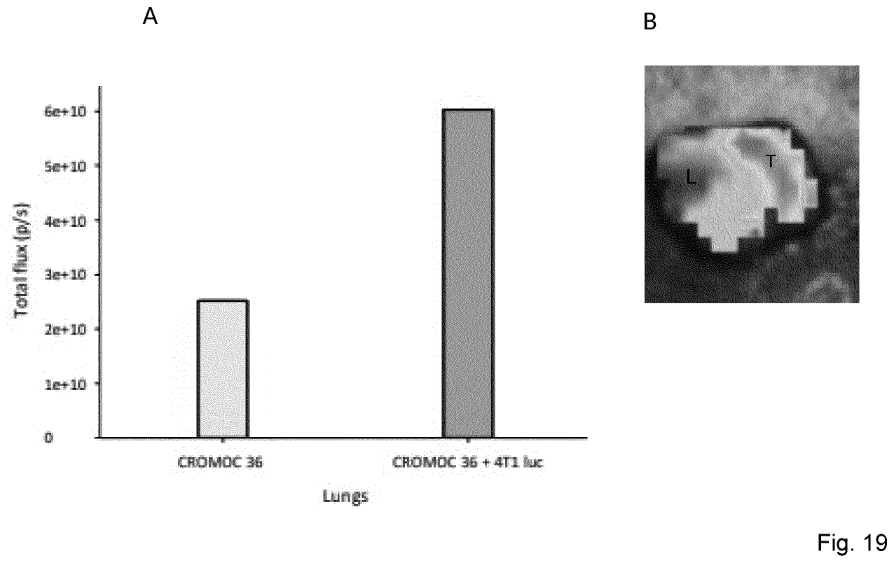

FIG. 19: In vivo organ-distribution of CROMOC36-AF680 molecules in tumor containing mice.

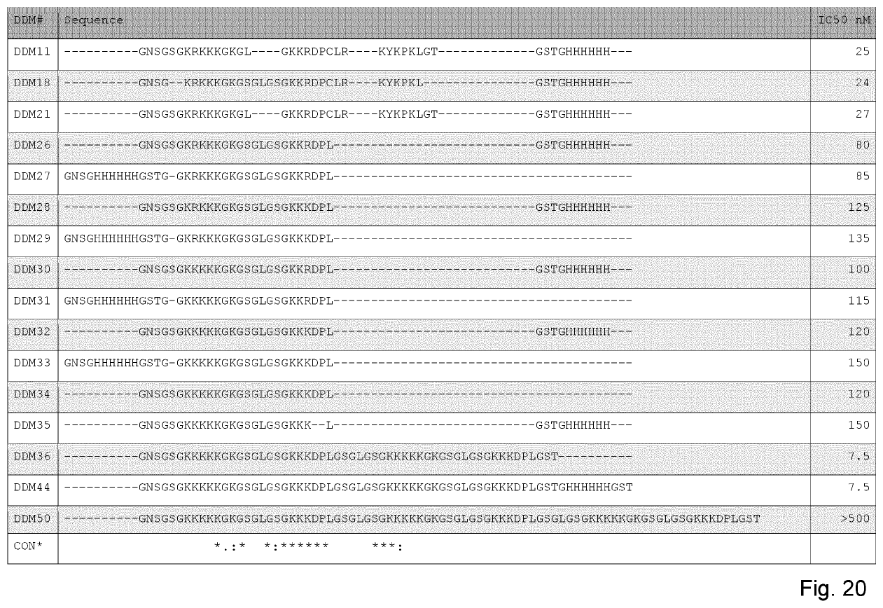

FIG. 20: CLUSTAL W (1.83) multiple sequence alignment of various DDMs Sequence Alignment of the DDM-CPP sub-family and IC50 comparison with the Endonuclease assay.

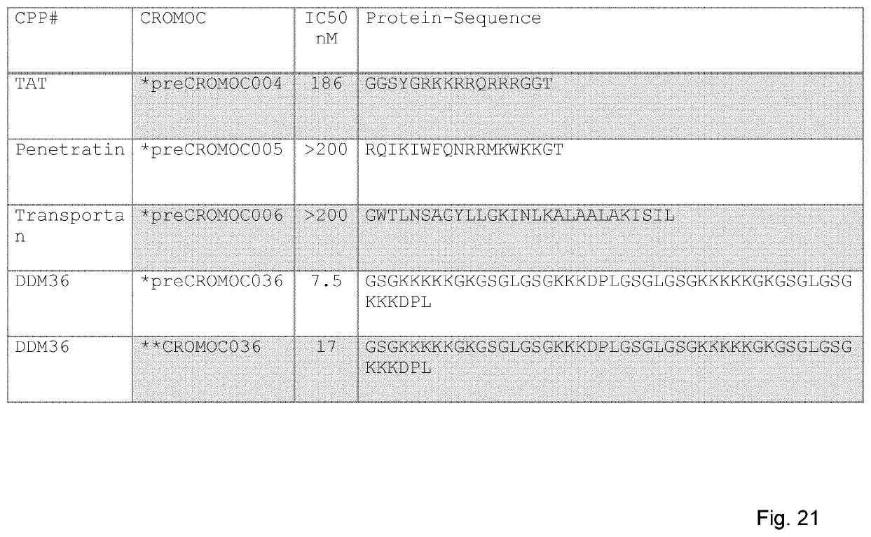

FIG. 21: Comparison of DDM36 with TAT, Pentratin, and Transportan.

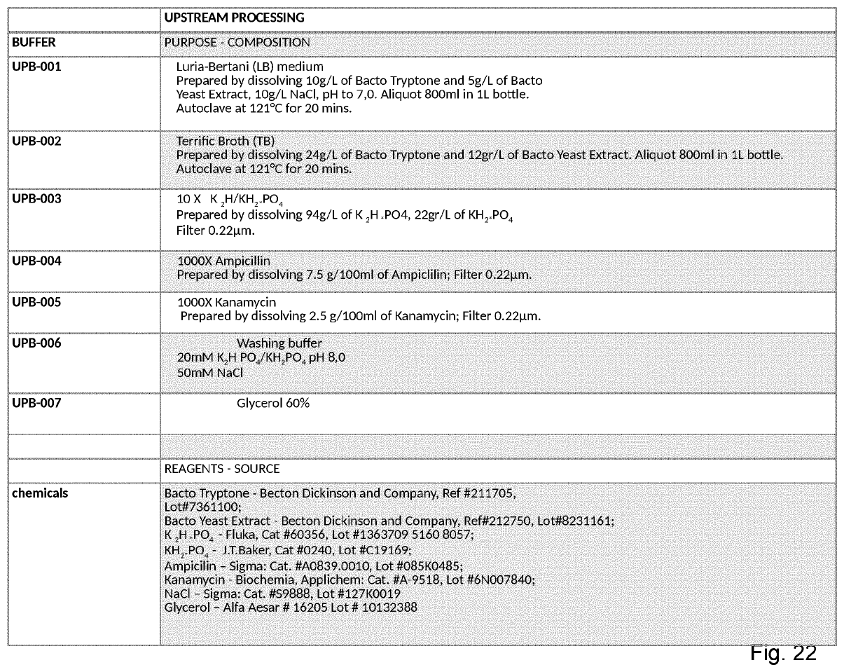

FIG. 22: UPB Buffers.

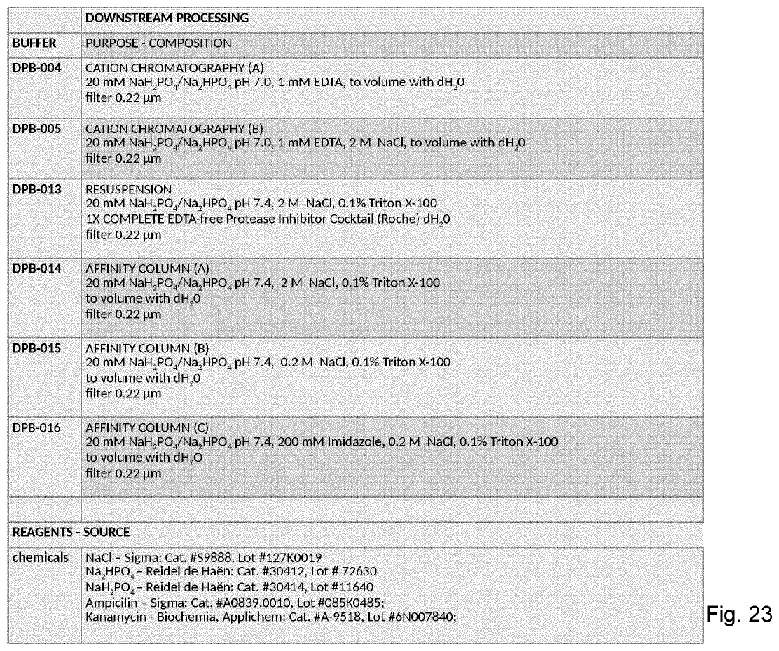

FIG. 23: UPB Buffers.

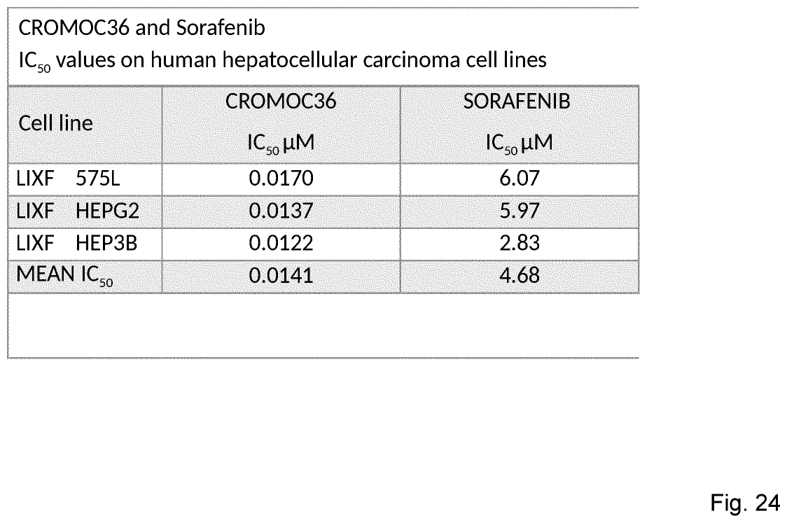

FIG. 24: IC₅₀ values of CROMOC36 and Sorafenib on human hepatocellular carcinoma cell lines.

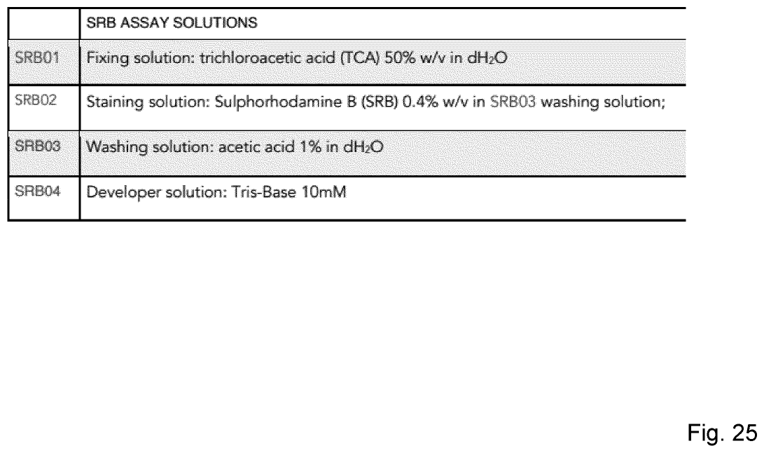

FIG. 25: SRB Buffers.

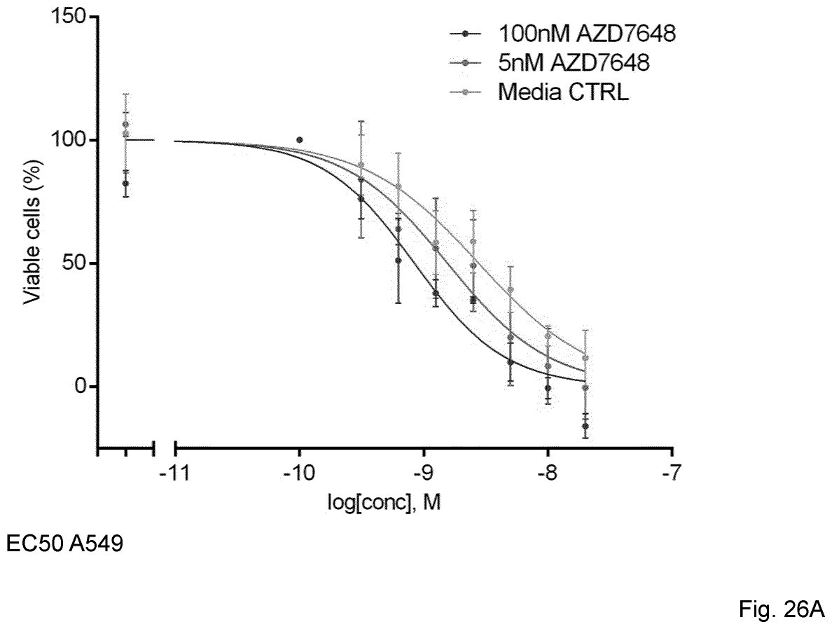

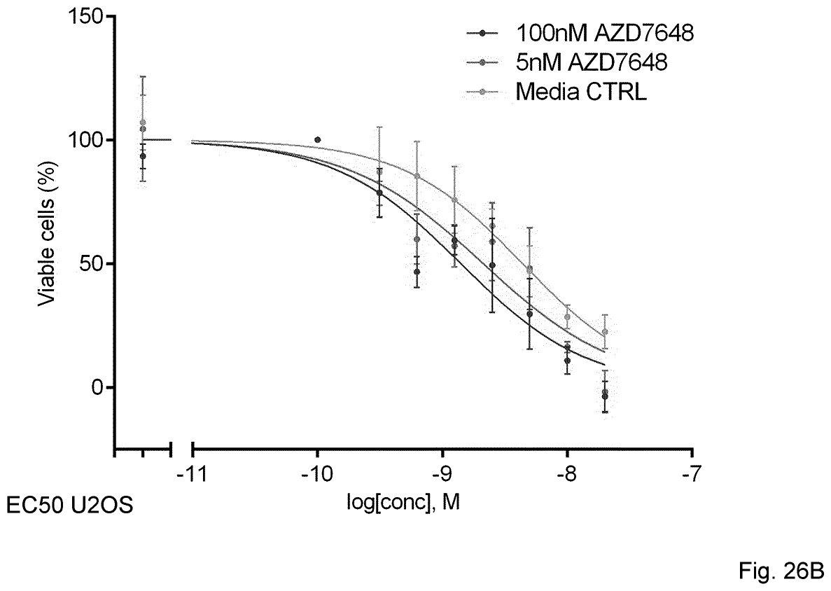

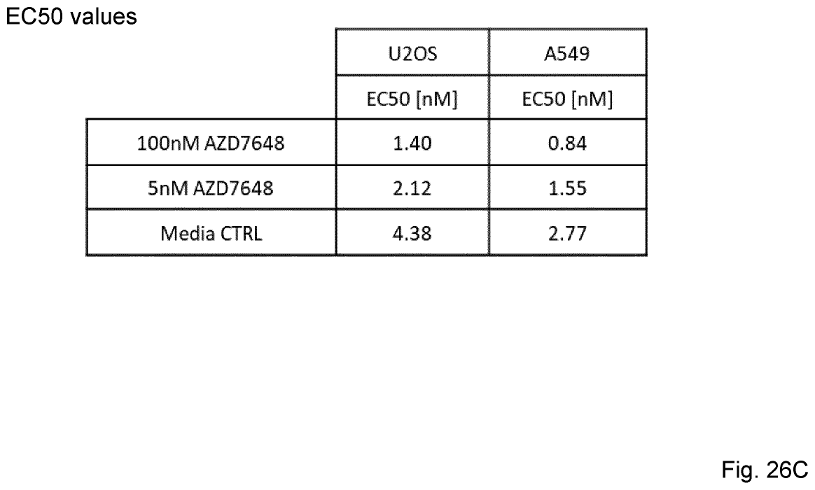

FIG. 26: Concentration dependent response to CROMOC62 in the presence of different concentrations of DNA-PK inhibitor AZD7648 was determined by SRB assay for a) U2-OS (A) and b) A 549 cells (B). Calculated EC50 values are indicated in C.

### EXAMPLES

### Example 1

For the development of a novel family of CPPs that shows efficiency at concentrations in the low nM range, are more stable in vitro and in vivo, do not interfere with solubility and allows for the development of drug like properties, synthetic CPPs were developed as Drug Delivery Modules (DDM). For this, different DDM sequences were analyzed in a structure activity relationship with using as a primary screening assay IC₅₀ values of a nuclease payload in cell-based assays. In more detail, DDM peptides were fused by recombinant techniques to the single chain PvuII Endonuclease polypeptide (preCROMOC) and analyzed by the Osteosarcoma (U2OS) cell based, 72 hours Monolayer Sulphorhodamine B (SRB) staining assay, as a measure for functional uptake of the preCROMOC-DDM fusion molecules. Results of successful developments are shown in FIG. 20. A comparison of the most suitable DDM36 with TAT, Pentratin, and Transportan showed orders of magnitude higher activity in the SRB assay (FIG. 21), different biodistribution (FIGS. 13-17) as well as higher stability was observed towards proteolytic degradation during molecule preparation.

### Example 2

Monolayer assays for in vitro testing of the IC50 activity of CROMOC compounds. The following cells were tested in the monolayer assays results are shown in FIGS. 2, 3, 4, and 24:

22RV1, 5637, 7860, A204, A375, A431, A549, A673, ACHN, ASPC1, BT20, BXPC3, CACO2, CAKIl, CALU6, CLS439, COLO205, COLO678, DLD1, DU145, EFO21, EJ28, HCT15, HEK293, CASKI, C33A, HELA, SAOS2, SKHEP1, LIXF575L, HEPG2, HEP3B, HS578T, HS729, HT1080, HT29, IGROVI, IMR90, J82, JAR, JEG3, JIMT1, LOVO, MCF7, MDAMB231, MDAMB435, MDAMB436, MDAMB468, U20S, MG63, MHHES1, MIAPACA2, MT3, NCIH292, NCIH358M, NCIH460, NCIH82, A2780, OVCAR3, OVCAR4, PANC1, PANC1005, PC3, PLC-PRF-5, RD, RDES, SF268, SF295, SKBR3, SKLMS1, SKMEL28, SKMEL5, SKNAS, SKNSH, SKOV3, SNB75, SW620, T24, TE671, U87MG, UMUC3, U031, and human Peripheral Blood Mononuclear Cell, PBMC. BEN; COLO-699; FU—OV-1; GI-LI-N; H-1339; HCC-15; HCC-44; HCC-827; HCT-116 p53+/+; HCT-116 p53−/−; HCT-116 p21−/−; primary Hepatocytes; IMR-32; LAN-5; LCLC-97TM1; LCLC-103H; ONCO-DG-1; SHSY-5Y; STA-NB3; STA-NB10; U2-OS; WI-38.

### Example 2a. Monolayer Assay Using Sulphorhodamine B (SRB) Staining

Cells were harvested from exponential phase cultures, and depending on the cell line 10.000 to 30.000 cells were plated in 96-well flat-bottom microtiter plates. After a 24 h recovery period to allow cells to resume exponential growth, supernatant was discarded and 100 μl of culture medium (eight control wells/plate) or culture medium with the test compound were added by a liquid handling robotic system (Microlab® Starlet, Hamilton) and treatment continued for 72 hrs. Compounds were applied at 9 concentrations eight wells/concentration). At least three independent experiments were performed. For SRB staining, cells were fixed with SRB01 for 1h at 4° C. (final concentration of TCA=10%) washed with a plate washer (Hydroflex, Tecan©) for five times with H₂O; and stained with 100 μl of SRB02 at room temperature for 10 min following 4 washes in SRB03 and finally 100 μl of developer solution SRB04 for 5 minutes with shaking. Optical density was assessed using a plate-reader (Sunrise, Tecan©) at 540 nm. Concentrations required for 50% inhibition of cell growth (IC₅₀) were calculated by non-linear regression (log[conc. of inhibitor] versus response (% T/C)) using the analysis software GraphPad Prism®, Prism 5 for windows, version 5.01 (GraphPad Software Inc., CA). (SRB Buffers FIG. 25).

### Example 2B

Monolayer Assay Propidium Iodid (PI). Cells were harvested from exponential phase cultures, counted and depending on the cell line 3.000 to 20.000 cells were plated in 96-well flat-bottom microtiter plates After a 24 h recovery period to allow cells to resume exponential growth, 10 μl of culture medium (four control wells/plate) or culture medium with the test compound were added by a liquid handling robotic system and treatment continued for four days. Compounds were applied in half-log increments at 10 concentrations in duplicate. Three independent experiments were performed. Cells were then washed with 200 μl PBS to remove dead cells followed by addition of 200 μl of a solution containing 7 μg/ml propidium iodide (PI) and 0.1% (v/v) Triton X-100. After an incubation period of 1-2 h at room temperature, fluorescence (FU) was measured using the Cytofluor 4000 microplate reader (excitation λ=530 nm, emission λ=620 nm) to quantify the amount of attached viable cells. Concentrations required for 50% inhibition of cell growth (IC₅₀) were calculated by non-linear regression (log[conc. of inhibitor]versus response (% T/C)) using the analysis software GraphPad Prism®, Prism 5 for windows, version 5.01 (GraphPad Software Inc., CA).

### Example 3

Expression of the Proteins of the Invention. Protein expression was obtained from the E. coli strains using a twoplasmid vector system as described in Ktihne C., et al. (NAR 31 (2003), 7227-7237). Cell growth for protein expression was done in 400 ml culture volume in a 2 L Erlenmeyer flask in a constitutive fashion and no antibiotics. Culture medium was inoculated at an optical cell density OD₆₀₀ of 0.008, with a freshly prepared pre-culture previously grown under Kanamycin and Ampicillin antibiotics selection (UPB-001, UPB-004, UPB-005). Growth medium was composed of Terrific Broth (TB, UPB-002) supplemented with UPB-003. Growth was at 210 RPM, 30° C., 16 hours in a Certomat BS-1 shaking incubator (Sartorious). After 16 hours growth an OD₆₀₀ of 6.7 (+/−0.2) were reached, pH 7.2 (+/−0.05) and E. coli cultures were centrifuged at 3557 g, +4° C. for 30 minutes. Pellets were then incubated on ice with 25 ml of washing buffer (UPB-006/pellet of 400 ml growth for 30 min), re-suspended and centrifuged again as above. Supernatants were discarded and wet biomass was measured. Under these conditions 400 ml of culture result in 5 g (+/−0.4 g) wet weight pellet. Storage of the wet pellet at −80° C. (UPB Buffers were described in FIG. 22).

### Example 4

Purification and PEG modification of the proteins of the invention. The following steps were done. Cell pellets disruption: frozen cell pellets stored at −80° C. (Example) were thawed in a water-bath at 30° C. for 10 minutes. Pellets were then resuspended in 7.5 ml of ice cold buffer DPB-013 per gram of cell pellet and then incubated for 30 minutes on ice. Homogenization: homogenizer GEA NS 1001L-2K (GEA Niro Soavi) was pre-equilibrated in ice-cold buffer DPB-013; cells were disrupted by 4 cycles at 1100 bar, keeping temperature at 4° C. Centrifugation: clarification of the homogenate was carried out by centrifugation for 1 hour at 25.000×g, 4° C. using a 3K30 centrifuge (Sigma). Sterile filtration: clarified supernatants were sterile filtered using a Stericup PVDF Durapore® 0.22 μm filtration device (Millipore). Affinity chromatography: sample loading was done at 79 cm/hr (20 ml/min), the rest of the chromatography at 159 cm/hr (40 ml/min); AKTA Purifier UPC 100, (GE Healthcare Bio-Sciences). Sterile filtered samples were loaded onto a pre-equilibrated (in buffer DPB-014) IMAC Sepharose 6 Fast Flow 50 ml (GE Healthcare Bio-Sciences AB). Wash out unbound protein for 10CV with DPB-014, then wash with DPB-015 to decrease salt concentration, elute with one step 100% DPB-016, 3CV. Anion exchange chromatography: all run was done at 150 cm/hr (20 ml/min); AKTA Purifier UPC 100, (GE Healthcare BioSciences AB,). Peak fraction from affinity chromatography elution was directly loaded onto pre-equilibrated (in buffer DPB-015) Fractogel EMD TMAE Medcap (M) 30 ml (Merck KGaA—Life Science Products—Darmstadt, Germany) and flow through was collected (the fraction that doesn't bind the column). Washed with 10 column volumes (CV) with DPB-015 elute with one step 100% DPB-014, 3 CV. (I) Cation exchange chromatography: all run was done at 197 cm/hr (50 ml/min); AKTA Purifier UPC 100, (GE Healthcare Bio-Sciences AB). Flow through fraction from anion exchange chromatography was loaded onto pre-equilibrated (in buffer DPB-004) Fractogel EMD SO3 (M) 40 ml (Merck KGaA—Life Science Products—Darmstadt, Germany), washed with 10 CV DPB-004 and eluted by a 3 step gradient, 25% buffer DPB-005 (C-terminal truncated SUB36 protein (theoretical mass 37928/37930 (amino-acid 2-331), molecular mass of 40113/40115 (aa 2-355) 40%; buffer DPB-005 (native full length SUB36 protein-compound, peak-fraction 43404.4/43406.8 (aa 2-387), 100% buffer DPB-005 (non-native isomeric forms). Protein peak fraction was frozen at −80° C. in sterile tubes. For the post-expression modification of the CROMOC molecules with PEG, Protein from −80 was thawed on ice (3 hours). Precipitates were cleared out by centrifugation 30 minutes, 25.000×g, 4° C. into 3K30 centrifuge (Sigma). Protein pool was prepared. N-terminal PEGylation Reaction: dilute native full length SUB36 protein-compound with 0.1 M NaH₂PO₄/Na₂HPO₄ pH 5.0 to 1 mg/ml and mixed with a 10× molar excess of mPEG-butyraldehyde 30 kDa (Laysan Bio Inc.) and a 1000× molar excess of NaBH₃CN (Sigma Aldrich). Incubation was carried out for 16 hours, 25° C., 125 rpm into CERTOMAT BS-1 incubator (Sartorius). The reaction was quenched by adding 30× molar excess glycine solution. (II) Cation exchange chromatography: all run was done at 157 cm/hr (40 ml/min); AKTA Purifier UPC 100, (GE Healthcare Bio-Sciences AB, Björkgatan 30, 75184 Uppsala, Sweden). Dilute the clarified supernatant 1:2.5 with buffer DPB-004. Sample was loaded onto pre-equilibrated (in buffer DPB-004) Fractogel EMD SO3 (M) 40 ml (Merck KGaA—Life Science Products—Darmstadt, Germany), wash 10 CV DPB-004, elute protein by a 3 step gradient, 20% buffer DPB-005 (poly-PEGylated-protein), 30% buffer DPB-005 (mono-PEGylated full length SUB36 protein-compound), 100% buffer DPB-005 (non-PEGylated protein). Microfiltration: mono-PEGylated full length SUB36 protein-compound was concentrated 1:10 by centrifugation & microfiltration (Vivaspin 15R, MWCO 30000, Sartorius) and subsequent 3 cycles washing in PBS. Storage at −80° C. UPB Buffers are described in FIG. 23. An Example of an SDS-gel of the various purification steps is shown in FIG. 1.

### Example 5

CROMOC36—in vitro activity Human hepatocellular carcinoma cell lines IC₅₀ values of CROMOC36 and Sorafenib, 96h treatment. CROMOC36 showed a concentration-dependent activity towards all cell lines tested with a geomean IC₅₀ of 0.0141 μM. The most sensitive cell line was HEP3B (IC₅₀=0.0122 μM), followed by HEPG2 (IC₅₀=0.0137 μM) and LIXF 575L (IC₅₀=0.017 μM). The individual results for CROMOC36 are included in FIG. 24. The reference compound Sorafenib displayed a geomean IC₅₀ of 4.68 μM for the three cell lines, indicating that CROMOC36 was over 300-fold more potent compared to Sorafenib. The most sensitive cell line towards Sorafenib was HEP3B (IC₅₀=2.83 μM). Nearly identical sensitivity was detected for HEPG2 (IC₅₀=5.97 p1M) and LIXF 575L (IC₅₀=6.07 μM). The individual results for Sorafenib are included in (FIG. 24). This study shows that the three HCC cell lines tested are sensitive to CROMOC36 with a geomean IC₅₀ of 0.0141 μM. CROMOC36 was over 300-fold more potent on these cell lines than the reference compound Sorafenib. These results suggest that CROMOC36 has a potent anticancer activity against HCC in vitro.

### Example 6

CROMOC36—in vitro activity Human pancreatic carcinoma cell lines. The aim of this study was to investigate the antitumor activity of CROMOC36 in vitro on a panel of 13 human pancreatic tumor cell lines. Antitumor activity was assessed after four days of CROMOC36 treatment using a propidium iodide based monolayer proliferation assay. Up to four experiments were performed in each cell line, testing CROMOC36 at ten concentrations in half-log increments up to 1 μM. Taxol, an antitumor agent commonly used for the treatment of pancreatic cancer, was used as a reference compound and tested in half-log steps up to 0.3 μM. CROMOC36 showed selective concentration-dependent antitumor activity towards the pancreatic cell lines tested with an overall geomean IC₅₀ value of 45.9 nM. The most sensitive cell lines were PATU8902 (IC₅₀=2.33 nM), PATU8988T (IC₅₀=2.79 nM) and PAXF 1657L (IC₅₀=4.73 nM), followed by PAXF 546L (IC₅₀=6.49 nM). In addition, MIAPACA2, HUPT3 and PANC1 also showed above-average sensitivity (IC₅₀˜10 nM). CROMOC36 showed no activity or only marginal activity on five of the cell lines tested. The reference compound Taxol showed concentration-dependent activity for all cell lines tested with a geomean IC₅₀ value of 1.99 nM. CROMOC36 showed selective and concentration-dependent antitumor activity towards the human pancreatic cell lines tested. CROMOC36 was most sensitive on PATU8902, PATU8988T, PAXF 1657L and PAXF 546L cell lines (FIG. 4).

### Example 7

To analyze the cytostatic activity in different tumor cell lines, the in vitro activity of CROMOC36 was analyzed in 80 different human tumor cell lines representing the mayor tumor indications by the Monolayer Sulphorhodamine B (SRB) staining assay for 72 hours. CROMOC36 shows anti-proliferative activity at nM concentrations in tumor cells from different indications (55/80 cell lines IC₅₀≤100 nM). This shows a broad cytostatic anti-tumor activity in various indications (FIG. 2). CROMOC36 shows antiproliferative activity at nM concentrations.

IC₅₀ range <0.5-19 nM. Notably, CROMOC36 is 40->100 times less cytotoxic to immortalized non-tumor cells than to cancer cells. SRB assay was done for 96 hours for various lung cancer, ovarian cancer and neuroblastoma and colon cancer cells and compared to non-tumor cells primary hepatocytes with of IC₅₀>10 PM.

### Example 9

A comparison of the uptake kinetics of PEGylated CROMOC36-AF680 (CROMOC36-AF680) versus non-PEGylated preCROMOC-AF680 was conducted. Fluorescence intensities were measured by Flow cytometry. U2OS cells were incubated for the indicated time points and cells were fixed after intensive washing of the surface bound molecules with ice cold PBS. The cellular uptake of PEGylated CROMOC36-AF680 is 6× slower than a non-PEGylated (preCROMOC36-AF680) as shown in FIG. 4.

### Example 10

CROMOC endocytosis inhibitor study to evaluate the pathways important for membrane crossing of CROMOC. U2OS cells were incubated with Fluorescein labeled-CROMOC36 at 4° C. (1) or in the presence of Cytocholasine D (2, CD); Chloropromazine (3, CPZ); Methyl-ßCyclodextrin(4, MB CD); Chloropromazine and Methyl-ß-Cyclodextrin(5, CPZ+MBCD); Heparin (5, HP) and cellular uptake was measured using flow cytometry. CD inhibits macropinozytosis; CPZ inhibits clathrin dependent endocytosis; MB CD inhibitor of caveolin dependent endocytosis's competes with the heparin receptor and 4° C. inhibits energy dependent endocytosis but not macropinocytosis or phagocytosis. X axis % CROMOC uptake inhibition. This example confirms that uptake of DDM CPPs is energy dependent, i.e. is not effective at 4° C. and is inhibited by CPZ as well es MB CD demonstrating clathrin as well as caveolin dependent endocytosis for DDM targeted transport and no micropinocytosis as summarized in FIG. 5. Similar results were obtained with different cell lines including colon cancer cells HCT116 or lung adenocarcinoma cell line A549.

### Example 11

A study was conducted to test the antitumor efficacy of CROMOC36 on an advanced human colon carcinoma with null p53 (HCT116 p53−/−) xenograft model and compare it to a vehicle or a nuclease impaired version CROMOC60 treatment group. CROMOC60 is no more catalytically active as a nuclease but is able to be transported to the nucleus of a cell. This study was also designed to proof that the active principle of the tumor growth inhibiting activity of CROMOC is the nuclease activity in vivo. CROMOC36 and CROMOC60 were both administered subcutaneously (s.c.) at 4 mg/kg dose on the days 0, 1, 2, 3, 6, 7, 8, 9, 10, 20, 22, 24. The study showed that progression of HCT116 p53−/− human colon carcinoma was significantly inhibited by CROMOC36 but not by CROMOC60 as compared to vehicle control. This demonstrates that the nuclease activity is responsible for the consistent anti-tumor effect seen in the mouse xenograft experiments. This also proofs that CROMOC36 can serve as a valid DNA DSB reagent.

A summary graph is shown in FIG. 7.

### Example 12

As a proof of the nuclease activity in vivo an Immunohistochemistry (IHC) analysis of paraffin embedded liver specimens of Male Sprague Dawley Rats after single IV treatment with CROMOC36. Samples were collected after 13-15 days and IHC was used to assess the Histone surrogate-markers for DNA double strand breaks Phospho-H2AX and Phospho-H3 for analyzing the persistence of DNA double stand breaks (DSBs) at very high CROMOC concentrations in tumors in comparison to the KI67marker.This proofs that DSBs are induced by the CROMOC molecules and that the DDM sequences can guide payloads into the nucleus of a cell in vivo. A representative example is shown in FIG. 8.

### Example 13

The efficacy of CROMOC36 on the Lewis lung carcinoma (LLC) model, was selected for testing the activity of CROMOC36 against primary tumor growth and metastasis formation. CROMOC36 was tested as a single agent and in combination with Cyclophosphamide (CYPP). In addition, single-agent CYPP was used as a reference compound, allowing comparison of the efficacy of CROMOC36 with a commonly used antitumor agent (summarized in FIG. 9). This study showed that murine LLC primary tumor growth was not inhibited after repeated subcutaneous treatment [five treatments every two days (q2dx5)] with CROMOC36 [15.2% tumor growth inhibition (TI)]. Conversely, treatment with CYPP did inhibit primary tumor growth in this model (78.6% TI). The simultaneous combination of CROMOC36 and CYPP gave a statistically significant (p=3×10⁻⁸) increase in inhibition of primary tumor growth (85.6% TI) compared to treatment with either of the compounds as single agents. In contrast to the primary tumor, CROMOC36 showed a strong antimetastatic effect: compared to controls, CROMOC36 (4 mg/kg, q2dx5) reduced the number of small, medium, and large metastases by 61.2%, 49.2% and 69.1%, respectively. Similar results were observed after treatment with 180 mg/kg CYPP. The combination of the two treatments further reduced the number of metastases, with a reduction of 81.1% for small metastases, 66.7% for medium metastases and 89.2% for large metastases.

Overall, CROMOC36 4 and 0.4 mg/kg inhibited the total number of metastases by 66% and 24%, respectively; CYPP inhibited the number of metastases by 62% and the combination of CROMOC36 (4 mg/kg) and CYPP (180 mg/kg) inhibited the number of metastases by 81%. Comparable inhibition of metastases volume was observed for all treatment groups.

No major issues were identified concerning tolerability of CROMOC36. The combination of CROMOC36 and CYPP did reduce body weight (BW) during the treatment schedule more than treatment with each as a single agent. However, BW recovered towards the end of the experiment resulting in a final decrease in BW of 3.7% (compared to the start of the experiment), which was comparable to the other treatment groups. This model clearly shows that CROMOC36 is highly active in orthotopic metastasis models but does not interfere with the intramuscular injected primary tumor. This contrasts with CYPP that is active in primary tumor and the effects seen in the metastasis stems from the fact that no primary tumor can evade to the metastatic site.

### Example 14

A study was conducted to evaluate the efficacy and tolerability of CROMOC36 against an HCT116 human colon carcinoma xenograft model with null p53 (HCT116 p53−/−) in female athymic nude mice. CROMOC36 was administered subcutaneously (s.c.) three times at intervals of two weeks (q14dx3) as a single agent or in combination with cisplatin (CDDP) at 4 mg/kg or vincristine (VCR) at 2 mg/kg which were both administered five times at intervals of one week (q7dx5). CROMOC36 was tested at 32 mg/kg or using a decreasing dosage of 32-16-16 mg/kg. CDDP and VCR were also administered as single agents to allow comparison of the efficacy of CROMOC36 with commonly used anticancer drugs. The dose and schedule of CDDP and VCR treatments remained the same when tested as single agents or in combination with CROMOC36. This study showed that progression of HCT116 p53−/− human colon carcinoma was inhibited by CROMOC36, both at the repeated concentration of 32 mg/kg (82.9% tumor growth inhibition; p<0.005 versus untreated control) and at the decreasing concentration of 32-16-16 mg/kg (91.6% tumor growth inhibition; p<0.005 versus untreated control) with better efficacy than single-agent CDDP (47.2% tumor growth inhibition) or VCR (45.4% tumor growth inhibition). The tumor growth inhibition observed when CROMOC36 32-16-16 mg/kg was administered in combination with CDDP or VCR did not change significantly with respect to treatment with single-agent CROMOC36. The inhibitory effect observed in the groups treated with CROMOC36 32-16-16 mg/kg, as a single agent or in combination with CDDP or VCR, persisted until day 50 (i.e. three weeks after the last treatment) with no significant decrease with respect to the tumor inhibition calculated on Day 31. Efficacy was also evaluated by measuring the tumor growth delay (TGD) and the number of regression responses. The control group reached the study endpoint (TTE), defined as a tumor volume of 1000 mm³, by Day 23. In all groups treated with CROMOC36, as a single agent or in combination with CDDP or VCR, the average tumor volume did not reach 1000 mm³. The TTE in these groups was >50 days, corresponding to a % TGD of >117%. In the CDDP and VCR groups, the TTE was 34 days (48% TGD) and 35 days (52% TGD), respectively. In addition, treatment with 32-16-16 mg/kg CROMOC36 resulted in one partial regression response and one tumor-free survivor. A graph is shown in FIG. 10.

### Example 15

The aim of this study was to assess the in vivo efficacy of CROMOC36 on a IMR32 human neuroblastoma xenograft model. CROMOC36 was administered subcutaneously (s.c.) at intervals of two weeks (q14dx2) at two different doses (32 mg/Kg) and also in combination with doxorubicin administered intravenously (i.v.) (32/kg CROMOC36+4+4 mg/Kg doxorubicin, s.c and i.v., q14dx2). Doxorubicin was also used alone as a reference compound (8 mg/kg, i.v., q14dx2). Vehicle was PBS. This study showed that growth of IMR32 human neuroblastoma was dramatically inhibited by CROMOC36 (32 mg/Kg), both alone and in combination with doxorubicin, in contrast efficacy from treatment with doxorubicin alone showed much lower efficiency (TGI 10%) compared to vehicle control. Treatment with CROMOC36 and CROMOC36+doxorubicin resulted in tumor growth inhibition of 82% and 89%, respectively, compared to controls.

In conclusion, these results show that CROMOC36 has strong antitumor activity at tolerated doses on a human IMR32 neuroblastoma model, both as a single agent and in combination with doxorubicin. These findings further support the in vivo efficacy of CROMOC36 on experimental tumor models after repeated s.c. treatments. Doxorubicin is accepted as the first line treatment in late Neuroblastoma and the superiority of CROMOC36 has significant implications for therapeutic use for Neuroblastoma.

A summary graph is shown in FIG. 11.

### Example 16

A M5076 ovarian reticulum sarcoma tumor cell line model was designed to test the antimetastatic activity in Liver allograft, i.v. Route (M5076). M5076 cells were maintained in vivo as ascitic tumor (by i.p. injection) in syngeneic female C57bl/6 mice. To obtain artificial hepatic metastasis, cells were recovered from peritoneal ascitic fluid, under sterile conditions, washed with PBS and injected into lateral tail vein (5×10⁴ live cells, in 200 μl PBS per mouse). Cell viability was evaluated by Trypan blue dye. Randomization criteria were: All injections of the cells were by the same person, mice were kept in cages for 7 days prior to cell injection and then remixed randomly into new cages; Mice were treated i.v., with 16 mg/kg CROMOC36 or PBS as vehicle once per week, for three times (q7dx3), starting from day 2 after tumor cell injection. Each treatment group consisted of 8 animals.

ANTIMETASTATIC ACTIVITY was measured at sacrifice (day 20^{th}), mice on group G1 (vehicle PBS), G2 (Doxorubicin 6 mg/kg (q4dx3) and G3 (CROMOC36 16 mg/kg (q7dx3)) were culled. Livers were harvested and fixed with Bouin's fixing solution (SIGMA-Aldrich). Surface metastatic nodules were evaluated by macroscopic observation under a magnifying glass. Determination of metastasis volume was performed by weighting each liver (FIG. 12B)

The efficacy of CROMOC36 and doxorubicin treatment on the reduction of hepatic metastasis formation was reported as percentage of inhibition (100−% T/C) comparing metastasis number and liver weight in all treated groups versus the control group. MEAN SURVIVAL TIME was evaluated in a second experiment. The three groups, treated in the same conditions as animals used for efficacy study on metastasis, were not sacrificed and were used to evaluate the Mean Survival Time (MST). Mice were examined daily, and severely moribund mice were culled to prevent unwanted suffering to the animals.

Survival time of each mouse was calculated as the number of days elapsed between tumor cell injection and death or euthanization.

Treatment efficacy was evaluated by comparing MST in treated mice to control group and expressed as % ILS.

Treatment was considered significant if % ILS >20%. As can be seen from FIG. 12 left panel ILS, CROMOC (68%) showed a significant higher survival time than the vehicle control (calculated as 0%) as well as Doxorubicin (32%) at MTD, considered the gold standard for this tumor model.

### Example 16a

In vivo imaging was done to compare bio-distribution in mice of CROMOC-DDM sequences with first generation CPPs engineered into the CROMOC payload. For this 8 mg of the CROMOC molecules of fluorescent labeled CROMOCs-AF680 (Alexafluor 680 (AF680)) containing various CPPs (TAT CROMOC04, Penetratin CROMOC 05, Transportan CROMOC06, and DDM36 CROMOC 36) were injected i.v. into athymic nude nu/nu (from balb/c wild type) female mice. CROMOCAF-680 in vivo localization in mice was monitored over time by bioluminescence analysis. Animals were anesthetized and the bladder was excluded from image recording. Fluorescence emitted from the CROMOC-AF680 fused to the described CPPs was detected using a cooled charge-coupled device camera, mounted on a lighttight specimen box (IVIS Lumina-II imaging system; Perkin-Elmer). Regions of interest from displayed images were quantified as total photon counts or photon/s using living Image® software (Perkin Elmer). Measurements were 2, 4, 6 and 24 hours after injection. Biodistributions varied significantly between the different CPPs. Whereas CROMOC36 exclusively localized to the liver, all the other molecules tested, exhibit distinct localization characteristics with a more common overall staining during life imaging. CROMOC04 shows whole body staining with signals also in the head region, and a strong liver fluorescence. CROMOC05 shows strong staining in the liver reminiscent of CROMOC36, but in addition distinct spots in the limbs and upper gut can be observed. For CROMOC006 overall whole-body staining was observed with only minor signal from the liver but a strong signal from the gut, signals in the head region and in the limbs. All the assessed CROMOC proteins described above were pegylated. Individual CPP localization characteristics were less pronounced in a non-PEGylated “pre-CROMOC” version showing that a post-expression modification leads to a more defined biodistribution and longer half-life of the molecules.

Examples are shown in FIG. 13 for CROMOC05, FIG. 14 for CROMOC06, FIG. 15 for CROMOC06, FIG. 16 for CROMOC36, FIG. 17 for preCROMOC36.

### Example 17

In vivo organ-distribution in healthy mice of CROMOC36 molecules. Four hours after i.v. injection of 8 mg/kg of the CROMOC molecule fluorescent labeled with the AF680 dye, organs (Liver, Kidney, Spleen, Gut, Heart Gall Bladder, Lungs) were isolated and fluorescence intensities were assessed ex vivo (total photon flux shown on X-axis logarithmic scale). FIG. 18 shows the average fluorescence intensities of the organs from three animals as depicted on the x-axis. These experiments show, that in healthy animals CROMOC36-AF680 predominantly distributes to the liver with three-fold more CROMOC than in the gut and six-fold more than in the gall bladder, kidney, lungs, and spleen. There was only residual signal detected in the heart, consistent with the observations in toxicology experiments and various tumor models were no cardiotoxic effects were observed. The later also confirms the exclusion of CROMOC36 from cardiac and skeletal muscle cells observed from the Lewis Lung allograft metastasis experiments in FIG. 9.

### Example 18

In vivo organ-distribution of CROMOC36-AF680 molecules in tumor containing mice. First orthotopic allograft 4T1 breast tumor lung metastasis mouse model was analyzed for CROMOC-AF680 distribution in vivo (FIG. 19A). Four hours after i.v. injection of 8 mg/kg of the CROMOC molecule labeled with the AF680 fluorescent dye, lungs containing 4T1cell metastasis were isolated and fluorescence intensity was assessed ex vivo (total photon flux shown on X-axis logarithmic scale). Whereas tumor free animals do not show significant retention of CROMOC36 molecules a more than 10.000× enrichment of CROMOC36 can be detected in breast tumor metastasis containing animals FIG. 19 A).

Tumor cell enrichment of CROMOC was further confirmed by experiments in liver were CROMOC enrichment in orthotopic allograft M5076 breast tumor metastasis were strongly enriched in comparison to the surrounding liver tissue. FIG. 19 B) shows an example were mice with M5076 liver metastasis were injected i.v with 8 mg/kg of CROMOC36-AF680 and fluorescence intensities were measured in the metastatic liver tumor (T) and compared to surrounding liver tissue (L) (FIG. 19B). Quantitative evaluation on three mice showed that the total photon flux in the tumor tissue was 2 to 3 orders of magnitude higher than in normal liver tissue proofing an enrichment of CROMOC in metastatic sites in the liver.

### Example 19: CROMOC62—In Vitro Activity in Combination with the Non Homologous End Joining (NHEJ) and V(D)J Repair Factor DNA Protein Kinase Inhibitor (DNA-PKi) AZD7648 (7-Methyl-2-[(7-methyl[1,2,4]triazolo[1,5-a]pyridin-6-yl)amino]-9-(tetrahydro-2H-pyran-4-yl)-7,9-dihydro-8H-purin-8-one) in osteosarcoma U2OS and in the lung carcinoma cell line A549

Concentration dependent response to CROMOC62 in the presence of different concentrations of DNA-PK inhibitor AZD7648 was determined by SRB assay for a) U2-OS (FIG. 26A) and b) A 549 cells (FIG. 26B). Calculated EC50 values are indicated in FIG. 26C. 2000 U2-OS (osteosarcoma cell line, grown in RPMI) or A549 cells (lung cancer cell line, grown in DMEM) were seeded on the day prior to treatment in 100 μl medium supplemented with 10% FCS and penicillin/streptomycin per 96 well. Indicated substances (CROMOC62 and AZD7648 at given concentration) were added in 50 μl medium, medium only served as negative control and 250 nM CROMOC62 served as positive control. After 70h cells were fixed for 1 h at 4° C. after addition of 37.5 μl 50% trichloroacetic acid. Fixed cells were washed 5 times with water, 100 μl 0.06% Sulforhodamine B in 1% acetic acid was added, incubated for 10 min at room temperature followed by 4 washing steps with 1% acetic acid. 100 μl 10 mM TRIS were added per well, shaken for 5 min and absorption was measured with a 560/20 nm filter, border wells were excluded. Normalization was performed using positive and negative control, EC50 calculation was carried out via Graphpad Prism 6 (Graph Pad Software, San Diego, CA) fit to the formula: (Y=100/(1+10\hat{ }((Log EC50−X)*HillSlope))). Sample size n=3, error bars indicate standard deviations.

Sequence Listing:
SEQ ID NO: 1:
X₀GX₁X₂GX₃X₄X₅GX₆X₇X₈GX₉X₁₀X₁₁X₁₂X₁₃X₁₄
SEQ ID NO: 2:
KKKKK
SEQ ID NO: 3:
KRKKK
SEQ ID NO: 4:
GLGS
SEQ ID NO: 5:
GSGL
SEQ ID NO: 6:
KYKPKL
SEQ ID NO: 7:
KYKPKLGT
SEQ ID NO: 8:
GX₃X₄X₅GX₆X₇X₈GX₉X₁₀
SEQ ID NO: 9:
GNSGSGKRKKKGKGLGKKRDPCLRKYKPKLGTGSTG
SEQ ID NO: 10:
GNSGSGKRKKKGKGLGKKRDPCLRKYKPKLGTGSTGHHHHHH
SEQ ID NO: 11:
GNSGKRKKKGKGSGLGSGKKRDPCLRKYKPKLGSTG
SEQ ID NO: 12:
GNSGKRKKKGKGSGLGSGKKRDPCLRKYKPKLGSTGHHHHHH
SEQ ID NO: 13:
GNSGSGKRKKKGKGLGKKRDPCLRKYKPKLGTGSTG
SEQ ID NO: 14:
GNSGSGKRKKKGKGLGKKRDPCLRKYKPKLGTGSTGHHHHHH
SEQ ID NO: 15:
GNSGSGKRKKKGKGSGLGSGKKRDPLGSTG
SEQ ID NO: 16:
GNSGSGKRKKKGKGSGLGSGKKRDPLGSTGHHHHHH
SEQ ID NO: 17:
GNSGGSTGGKRKKKGKGSGLGSGKKRDPL
SEQ ID NO: 18:
GNSGHHHHHHGSTGGKRKKKGKGSGLGSGKKRDPL
SEQ ID NO: 19:
GNSGSGKRKKKGKGSGLGSGKKKDPLGSTG
SEQ ID NO: 20:
GNSGSGKRKKKGKGSGLGSGKKKDPLGSTGHHHHHH
SEQ ID NO: 21:
GNSGGSTGGKRKKKGKGSGLGSGKKKDPL
SEQ ID NO: 22:
GNSGHHHHHHGSTGGKRKKKGKGSGLGSGKKKDPL
SEQ ID NO: 23:
GNSGSGKKKKKGKGSGLGSGKKRDPLGSTG
SEQ ID NO: 24:
GNSGSGKKKKKGKGSGLGSGKKRDPLGSTGHHHHHH
SEQ ID NO: 25:
GNSGGSTGGKKKKKGKGSGLGSGKKRDPL
SEQ ID NO: 26:
GNSGHHHHHHGSTGGKKKKKGKGSGLGSGKKRDPL
SEQ ID NO: 27:
GNSGSGKKKKKGKGSGLGSGKKKDPLGSTG
SEQ ID NO: 28:
GNSGSGKKKKKGKGSGLGSGKKKDPLGSTGHHHHHH
SEQ ID NO: 29:
GNSGGSTGGKKKKKGKGSGLGSGKKKDPL
SEQ ID NO: 30:
GNSGHHHHHHGSTGGKKKKKGKGSGLGSGKKKDPL
SEQ ID NO: 31:
GNSGSGKKKKKGKGSGLGSGKKKLGSTG
SEQ ID NO: 32:
GNSGSGKKKKKGKGSGLGSGKKKLGSTGHHHHHH
SEQ ID NO: 33:
GNSGSGKKKKKGKGSGLGSGKKKDPLGSGLGSGKKKKKGKGSGLGSGKKKDPLGSTGGST
SEQ ID NO: 34:
GNSGSGKKKKKGKGSGLGSGKKKDPL
SEQ ID NO: 35:
GNSGSGKKKKKGKGSGLGSGKKKDPLGSGLGSGKKKKKGKGSGLGSGKKKDPLGSTGHHHHHHG
ST
SEQ ID NO: 36:
GNSGSGKKKKKGKGSGLGSGKKKDPLGSGLGSGKKKKKGKGSGLGSGKKKDPLGST
SEQ ID NO: 37:
SHPDLNKLLELWPHIQEYQDLALKHGINDIFQDNGGKLLQVLLITGLTVLPGREGNDAVDNAGQ
EYELKSINIDLTKGESTHHHMNPVIIAKYRQVPWIFAIYRGIAIEAIYRLEPKDL-
EFYYDKWERKWYSDGHKDINNPKIPVKYVMEHGTKIY
SEQ ID NO: 38:
SHPDLNKLLELWPHIQEYQDLALKHGINDIFQDNGGKLLQVLLITGLTVLPGREGNDAVDNAGQ
EYELKSINIDLTKGFSTHHHMNPVIIAKYRQVPWIFAIYRGIAIEAIYRLEPKDLEFYYDKWER
KWYSDGHKDINNPKIPVKYVMEHGTKIYGSGG
SEQ ID NO: 39:
SGGSSHPDLNKLLELWPHIQEYQDLALKHGINDIFQDNGGKLLQVLLITGLTVLPGREGNDAVD
NAGQEYELKSINIDLTKGESTHHHMNPVIIAKYRQVPWIFAIYRGIAIEAIYRLEPKDLEFYYD
KWERKWYSDGHKDINNPKIPVKYVMEHGTKIY
SEQ ID NO: 40:
SHPDLNKLLELWPHIQEYQDLALKHGINDIFQDNGGKLLQVLLITGLTVLPGREGNDAVDNAGQ
EYELKSINIDLTKGFSTHHHMNPVIIAKYRQVPWIFAIYRGIAIEAIYRLEPKDLEFYYDKWER
KWYSDGHKDINNPKIPVKYVMEHGTKIYGNSG
SEQ ID NO: 41:
MSHPDLNKLLELWPHIQEYQDLALKHGINDIFQDNGGKLLQVLLITGLTVLPGREGNDAVDNAG
QEYELKSINIDLTKGFSTHHHMNPVIIAKYRQVPWIFAIYRGIAIEAIYRLEPKDLEFYYDKWE
RKWYSDGHKDINNPKIPVKYVMEHGTKIY
SEQ ID NO: 42:
SHPDLNKLLELWPHIQEYQDLALKHGINDIFQDNGGKLLQVLLITGLTVLPGREGNDAVDNAGQ
EYELKSINIDLTKGFSTHHHMNPVIIAKYRQVPWIFAIYRGIAIEAIYRLEPKDLEFYYDKWER
KWYSDGHKDINNPKIPVKYVMEHGTKIYGSGGC
SEQ ID NO: 43:
CSGGSSHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKDLEFYY
DKWERKWYSDGHKDINNPKIPVKYVMEHGTKIY
SEQ ID NO: 44:
MSHPDLNKLLELWPHIQEYQDLALKHGINDIFQDNGGKLLQVLLITGLTVLPGREGNDAVDNAG
QEYELKSINIDLTKGFSTHHHMNPVIIAKYRQVPWIFAIYRGIAIEAIYRLEPKDLEFYYDKWE
RKWYSDGHKDINNPKIPVKYVMEHGTKIYGSGGCSGGSSHPDLNKLLELWPHIQEYQDLALKHG
INDIFQDNGGKLLQVLLITGLTVLPGREGNDAVDNAGQEYELKSINIDLTKGFSTHHHMNPVII
AKYRQVPWIFAIYRGIAIEAIYRLEPKDLEFYYDKWERKWYSDGHKDINNPKIPVKYVMEHGTK
IY
SEQ ID NO: 45:
MSHPDLNKLLELWPHIQEYQDLALKHGINDIFQDNGGKLLQVLLITGLTVLPGREGNDAVDNAG
QEYELKSINIDLTKGFSTHHHMNPVIIAKYRQVPWIFAIYRGIAIEAIYRLEPKDLEFYYDKWE
RKWYSDGHKDINNPKIPVKYVMEHGTKIYGSGGSGGSSHPDLNKLLELWPHIQEYQDLALKHGI
NDIFQDNGGKLLQVLLITGLTVLPGREGNDAVDNAGQEYELKSINIDLTKGFSTHHHMNPVIIA
KYRQVPWIFAIYRGIAIEAIYRLEPKDLEFYYDKWERKWYSDGHKDINNPKIPVKYVMEHGTKI
Y
SEQ ID NO: 46:
MSHPDLNKLLELWPHIQEYQDLALKHGINDIFQDNGGKLLQVLLITGLTVLPGREGNDAVDNAG
QEYELKSINIDLTKGFSTHHHMNPVIIAKYRQVPWIFAIYRGIAIEAIYRLEPKDLEFYYDKWE
RKWYSDGHKDINNPKIPVKYVMEHGTKIYGSGGSGGS
SEQ ID NO: 47:
SHPDLNKLLELWPHIQEYQDLALKHGINDIFQDNGGKLLQVLLITGLTVLPGREGNDAVDNAGQ
EYELKSINIDLTKGFSTHHHMNPVIIAKYRQVPWIFAIYRGIAIEAIYRLEPKDLEFYYDKWER
KWYSDGHKDINNPKIPVKYVMEHGTKIYGSGGSGGS
SEQ ID NO: 48:
GSGGSGGSSHPDLNKLLELWPHIQEYQDLALKHGINDIFQDNGGKLLQVLLITGLTVLPGREGN
DAVDNAGQEYELKSINIDLTKGFSTHHHMNPVIIAKYRQVPWIFAIYRGIAIEAIYRLEPKDLE
FYYDKWERKWYSDGHKDINNPKIPVKYVMEHGTKIY
SEQ ID NO: 49:
MSHPDLNKLLELWPHIQEYQDLALKHGINDIFQDNGGKLLQVLLITGLTVLPGREGNDAVDNAG
QEYELKSINIDLTKGFSTHHHMNPVIIAKYRQVPWIFAIYRGIAIEAIYRLEPKDLEFYYDKWE
RKWYSDGHKDINNPKIPVKYVMEHGTKIYGSGGSGGSSHPDLNKLLELWPHIQEYQDLALKHGI
NDIFQDNGGKLLQVLLITGLTVLPGREGNDAVDNAGQEYELKSINIDLTKGFSTHHHMNPVIIA
KYRQVPWIFAIYRGIAIEAIYRLEPKDLEFYYDKWERKWYSDGHKDINNPKIPVKYVMEHGTKI
YGNSGSGKKKKKGKGSGLGSGKKKDPLGSGLGSGKKKKKGKGSGLGSGKKKDPLGST
(CROMOC36)
SEQ ID NO: 60:
MSHPDLNKLLELWPHIQEYQDLALKHGINDIFQDNGGKLLQVLLITGLTVLPGREGNDAVDNAG
QEYELKSINIDLTAGFSTHHHMNPVIIAKYRQVPWIFAIYRGIAIEAIYRLEPKDLEFYYDKWE
RKWYSDGHKDINNPKIPVKYVMEHGTKIYGSGGSGGSSHPDLNKLLELWPHIQEYQDLALKHGI
NDIFQDNGGKLLQVLLITGLTVLPGREGNDAVDNAGQEYELKSINIDLTAGFSTHHHMNPVIIA
KYRQVPWIFAIYRGIAIEAIYRLEPKDLEFYYDKWERKWYSDGHKDINNPKIPVKYVMEHGTKI
YGNSGSGKKKKKGKGSGLGSGKKKDPLGSGLGSGKKKKKGKGSGLGSGKKKDPLGST
(CROMOC60)
SEQ ID NO: 62:
MSHPDLNKLLELWPHIQEYQDLALKHGINDIFQDNGGKLLQVLLITGLTVLPGREGNDAVDNAG
QEYELKSINIDLTKGFSTHHHMNPVIIAKYRQVPWIFAIYRGIAIEAIYRLEPKDLEFYYDKWE
RKWYSDGHKDINNPKIPVKYVMEHGTKIYGSGGCSGGSSHPDLNKLLELWPHIQEYQDLALKHG
INDIFQDNGGKLLQVLLITGLTVLPGREGNDAVDNAGQEYELKSINIDLTKGESTHHHMNPVII
AKYRQVPWIFAIYRGIAIEAIYRLEPKDLEFYYDKWERKWYSDGHKDINNPKIPVKYVMEHGTK
IYGNSGSGKKKKKGKGSGLGSGKKKDPLGSGLGSGKKKKKGKGSGLGSGKKKDPLGST
(CROMOC62)
(CP63)
SHPDLNKLLELWPHIQEYQDLALKHGINDIFQDNGGKLLQVLLITGLTVLPGREGNDAVDNAGQ
EYELKSINIDLTKGFSTHHHMNPVIIAKYRQVPWIFAIYRGIAIEAIYRLEPKDLEFYYDKWER
KWYSDGHKDINNPKIPVKYVMEHGTKIYGSGGCCSGGSSHPDLNKLLELWPHIQEYQDLALKHG
INDIFQDNGGKLLQVLLITGLTVLPGREGNDAVDNAGQEYELKSINIDLTKGFSTHHHMNPVII
AKYRQVPWIFAIYRGIAIEAIYRLEPKDLEFYYDKWERKWYSDGHKDINNPKIPVKYVMEHGTK
IY
(CP70)
SHPDLNKLLELWPHIQEYQDLALKHGINDIFQDNGGKLLQVLLITGLTVLPGREGNDAVDNAGQ
EYELASINIDLTKGFSTHHHMNPVIIAKYRQVPWIFAIYRGIAIEAIYRLEPKDLEFYYDKWER
KWYSDGHKDINNPKIPVKYVMEHGTKIYGSGGCSGGSSHPDLNKLLELWPHIQEYQDLALKHGI
NDIFQDNGGKLLQVLLITGLTVLPGREGNDAVDNAGQEYELASINIDLTKGFSTHHHMNPVIIA
KYRQVPWIFAIYRGIAIEAIYRLEPKDLEFYYDKWERKWYSDGHKDINNPKIPVKYVMEHGTKI
Y
(CP87)
SHPDLNKLLELWPHIQEYQDLALKHGINDIFQDNGGKLLQVLLITGLTVLPGREGNDAVDNAGQ
EYELASINIDLTKGFSTHHHMNPVIIAKYRQVPWIFAIYRGIAIEAIYRLEPKDLEFYYDKWER
KWYSDGHKDINNPKIPVKYVMEHGTKIYGSGGCCSGGSSHPDLNKLLELWPHIQEYQDLALKHG
INDIFQDNGGKLLQVLLITGLTVLPGREGNDAVDNAGQEYELASINIDLTKGESTHHHMNPVII
AKYRQVPWIFAIYRGIAIEAIYRLEPKDLEFYYDKWERKWYSDGHKDINNPKIPVKYVMEHGTK
IY
(CP88)
SHPDLNKLLELWPHIQEYQDLALKHGINDIFQDNGGKLLQVLLITGLTVLPGREGNDAVDNAGQ
EYELKSINIDLTKGFSTHHHMNPVIIAKYRQVPWIFAIYRGIAIEAIYRLEPKDLEFYYDKWER
KWYSDGHKDINNPKIPVKYVMEHGTKIYGSGGCCCSGGSSHPDLNKLLELWPHIQEYQDLALKH
GINDIFQDNGGKLLQVLLITGLTVLPGREGNDAVDNAGQEYELKSINIDLTKGFSTHHHMNPVI
IAKYRQVPWIFAIYRGIAIEAIYRLEPKDLEFYYDKWERKWYSDGHKDINNPKIPVKYVMEHGT
KIY
(CP89)
SHPDLNKLLELWPHIQEYQDLALKHGINDIFQDNGGKLLQVLLITGLTVLPGREGNDAVDNAGQ
EYELASINIDLTKGESTHHHMNPVIIAKYRQVPWIFAIYRGIAIEAIYRLEPKDLEFYYDKWER
KWYSDGHKDINNPKIPVKYVMEHGTKIYGSGGCCCSGGSSHPDLNKLLELWPHIQEYQDLALKH
GINDIFQDNGGKLLQVLLITGLTVLPGREGNDAVDNAGQEYELASINIDLTKGFSTHHHMNPVI
IAKYRQVPWIFAIYRGIAIEAIYRLEPKDLEFYYDKWERKWYSDGHKDINNPKIPVKYVMEHGT
KIY
(CROMOC63)
SHPDLNKLLELWPHIQEYQDLALKHGINDIFQDNGGKLLQVLLITGLTVLPGREGNDAVDNAGQ
EYELKSINIDLTKGESTHHHMNPVIIAKYRQVPWIFAIYRGIAIEAIYRLEPKDLEFYYDKWER
KWYSDGHKDINNPKIPVKYVMEHGTKIYGSGGCCSGGSSHPDLNKLLELWPHIQEYQDLALKHG
INDIFQDNGGKLLQVLLITGLTVLPGREGNDAVDNAGQEYELKSINIDLTKGFSTHHHMNPVII
AKYRQVPWIFAIYRGIAIEAIYRLEPKDLEFYYDKWERKWYSDGHKDINNPKIPVKYVMEHGTK
IYGNSGSGKKKKKGKGSGLGSGKKKDPLGSGLGSGKKKKKGKGSGLGSGKKKDPLGST
(CROMOC70)
SHPDLNKLLELWPHIQEYQDLALKHGINDIFQDNGGKLLQVLLITGLTVLPGREGNDAVDNAGQ
EYELASINIDLTKGFSTHHHMNPVIIAKYRQVPWIFAIYRGIAIEAIYRLEPKDLEFYYDKWER
KWYSDGHKDINNPKIPVKYVMEHGTKIYGSGGCSGGSSHPDLNKLLELWPHIQEYQDLALKHGI
NDIFQDNGGKLLQVLLITGLTVLPGREGNDAVDNAGQEYELASINIDLTKGFSTHHHMNPVIIA
KYRQVPWIFAIYRGIAIEAIYRLEPKDLEFYYDKWERKWYSDGHKDINNPKIPVKYVMEHGTKI
YGNSGSGKKKKKGKGSGLGSGKKKDPLGSGLGSGKKKKKGKGSGLGSGKKKDPLGST
(CROMOC87)
SHPDLNKLLELWPHIQEYQDLALKHGINDIFQDNGGKLLQVLLITGLTVLPGREGNDAVDNAGQ
EYELASINIDLTKGFSTHHHMNPVIIAKYRQVPWIFAIYRGIAIEAIYRLEPKDLEFYYDKWER
KWYSDGHKDINNPKIPVKYVMEHGTKIYGSGGCCSGGSSHPDLNKLLELWPHIQEYQDLALKHG
INDIFQDNGGKLLQVLLITGLTVLPGREGNDAVDNAGQEYELASINIDLTKGFSTHHHMNPVII
AKYRQVPWIFAIYRGIAIEAIYRLEPKDLEFYYDKWERKWYSDGHKDINNPKIPVKYVMEHGTK
IYGNSGSGKKKKKGKGSGLGSGKKKDPLGSGLGSGKKKKKGKGSGLGSGKKKDPLGST
(CROMOC88)
SHPDLNKLLELWPHIQEYQDLALKHGINDIFQDNGGKLLQVLLITGLTVLPGREGNDAVDNAGQ
EYELKSINIDLTKGFSTHHHMNPVIIAKYRQVPWIFAIYRGIAIEAIYRLEPKDLEFYYDKWER
KWYSDGHKDINNPKIPVKYVMEHGTKIYGSGGCCCSGGSSHPDLNKLLELWPHIQEYQDLALKH
GINDIFQDNGGKLLQVLLITGLTVLPGREGNDAVDNAGQEYELKSINIDLTKGFSTHHHMNPVI
IAKYRQVPWIFAIYRGIAIEAIYRLEPKDLEFYYDKWERKWYSDGHKDINNPKIPVKYVMEHGT
KIYGNSGSGKKKKKGKGSGLGSGKKKDPLGSGLGSGKKKKKGKGSGLGSGKKKDPLGST
(CROMOC89)
SHPDLNKLLELWPHIQEYQDLALKHGINDIFQDNGGKLLQVLLITGLTVLPGREGNDAVDNAGQ
EYELASINIDLTKGFSTHHHMNPVIIAKYRQVPWIFAIYRGIAIEAIYRLEPKDLEFYYDKWER
KWYSDGHKDINNPKIPVKYVMEHGTKIYGSGGCCCSGGSSHPDLNKLLELWPHIQEYQDLALKH
GINDIFQDNGGKLLQVLLITGLTVLPGREGNDAVDNAGQEYELASINIDLTKGFSTHHHMNPVI
IAKYRQVPWIFAIYRGIAIEAIYRLEPKDLEFYYDKWERKWYSDGHKDINNPKIPVKYVMEHGT
KIYGNSGSGKKKKKGKGSGLGSGKKKDPLGSGLGSGKKKKKGKGSGLGSGKKKDPLGST

1. Compound comprising or consisting of a polypeptide with the general formula (I):
X₀GX₁X₂GX₃X₄X₅GX₆X₇X₈GX₉X₁₀X₁₁X₁₂X₁₃X₁₄,
wherein
G is glycine;
X₀ is present or not and, if present, is an amino acid linker;
X₁ is N or S, wherein N is asparagine and S is serine;
X₂ is S or T, wherein S is serine and T is threonine;
X₃ is present or not and, if present, is S, wherein S is serine;
X₄ is present or not and, if present, is G, wherein G is glycine;
X₅ is a basic polypeptide stretch consisting of 5 basic amino acid residues selected from R and K,
wherein R is arginine and K is lysine, preferably wherein X₅ comprises at least 3 K amino acid residues, especially wherein X₅ is KKKKK or KRKKK;
X₆ is a basic amino acid residue selected from R and K, wherein R is arginine and K is lysine, preferably wherein X₆ is K;
X₇ is S or L, wherein S is serine and L is leucine;
X₈ is present or not and, if present, is a polypeptide stretch consisting of the amino acid sequence GLGS, wherein G is glycine, L is leucine and S is serine;
X₉ is a basic polypeptide stretch consisting of 3 basic amino acid residues selected from R and K, wherein R is arginine and K is lysine, preferably wherein X₉ comprises at least 2 K amino acid residues, especially wherein X₉ is KKK or KKR;
X₁₀ is present or not and, if present, is L or a polypeptide stretch consisting of the amino acid sequence DPL or DPC, wherein L is leucine, D is aspartic acid, P is proline, and C is cysteine;
X₁₁ is present or not and, if present, is a polypeptide stretch consisting of the amino acid sequence LR or GSGL, wherein L is leucine, R is arginine, G is glycine, and S is serine;
X₁₂ is present or not and, if present, is a basic polypeptide stretch with at least 20% basic amino acid residues selected from K and R and at least a P residue, preferably wherein X₁₂ is selected from KYKPKL, KYKPKLGT, or GX₃X₄X₅GX₆X₇X₈GX₉X₁₀, wherein K, R, P, L, G, T, X₃, X₄, X₅, GX₆, X₇, X₈, GX₉, and X₁₀, are defined as above and wherein Y is tyrosine;
X₁₃ is present or not and, if present, is a polypeptide stretch consisting of the amino acid sequences GS, GST, GST, GSG, GSTG, or GSGL, wherein G, S, T and L are defined as above;
X₁₄ is present or not and, if present, is an amino acid linker;
wherein the polypeptide has a length from 20 to 75 amino acid residues, preferably from 24 to 65 amino acid residues, especially from 25 to 60 amino acid residues.

2. Compound according to claim 1, wherein X₁ is N, X₂ is S, X₃ is S, X₄ is G, X₅ is KKKKK or KRKKK, X₆ is K, X₉ is KKK or KKR, and X₁₀ is L or a polypeptide stretch consisting of the amino acid sequence DPL or DPC, wherein N, S, G, R, L, D, P and C are defined as above.

3. Compound according to claim 1, wherein the compound is selected from the group
GNSGSGKRKKKGKGLGKKRDPCLRKYKPKLGTGSTG(HHHHHH)
(DDM11),
GNSGKRKKKGKGSGLGSGKKRDPCLRKYKPKLGSTG(HHHHHH)
(DDM18),
GNSGSGKRKKKGKGLGKKRDPCLRKYKPKLGTGSTG(HHHHHH)
(DDM21),
GNSGSGKRKKKGKGSGLGSGKKRDPLGSTG(HHHHHH)
(DDM26),
GNSG(HHHHHH)GSTGGKRKKKGKGSGLGSGKKRDPL
(DDM27),
GNSGSGKRKKKGKGSGLGSGKKKDPLGSTG(HHHHHH)
(DDM28),
GNSG(HHHHHH)GSTGGKRKKKGKGSGLGSGKKKDPL
(DDM29),
GNSGSGKKKKKGKGSGLGSGKKRDPLGSTG(HHHHHH)
(DDM30),
GNSG(HHHHHH)GSTGGKKKKKGKGSGLGSGKKRDPL
(DDM31),
GNSGSGKKKKKGKGSGLGSGKKKDPLGSTG(HHHHHH)
(DDM32),
GNSG(HHHHHH)GSTGGKKKKKGKGSGLGSGKKKDPL
(DDM33),
GNSGSGKKKKKGKGSGLGSGKKKDPL
(DDM34),
GNSGSGKKKKKGKGSGLGSGKKKLGSTG(HHHHHH)
(DDM35),
GNSGSGKKKKKGKGSGLGSGKKKDPLGSGLGSGKKKKKGKGSGLGS
GKKKDPLGST
(DDM36),
and
GNSGSGKKKKKGKGSGLGSGKKKDPLGSGLGSGKKKKKGKGSGLGSG
KKKDPLGSTG(HHHHHH)GST
(DDM44),
wherein (HHHHHH; a histidine tag)may be present
or not; preferably
GNSGSGKRKKKGKGLGKKRDPCLRKYKPKLGTGSTG
(DDM11),
GNSGKRKKKGKGSGLGSGKKRDPCLRKYKPKLGSTG
(DDM18),
GNSGSGKRKKKGKGLGKKRDPCLRKYKPKLGTGSTG
(DDM21),
GNSGSGKRKKKGKGSGLGSGKKRDPLGSTG
(DDM26),
GNSGGSTGGKRKKKGKGSGLGSGKKRDPL
(DDM27),
GNSGSGKRKKKGKGSGLGSGKKKDPLGSTG
(DDM28),
GNSGGSTGGKRKKKGKGSGLGSGKKKDPL
(DDM29),
GNSGSGKKKKKGKGSGLGSGKKRDPLGSTG
(DDM30),
GNSGGSTGGKKKKKGKGSGLGSGKKRDPL
(DDM31),
GNSGSGKKKKKGKGSGLGSGKKKDPLGSTG
(DDM32),
GNSGSGKKKKKGKGSGLGSGKKKDPL
(DDM34),
GNSGSGKKKKKGKGSGLGSGKKKDPLGSGLGSGKKKKKGKGSGL
GSGKKKDPLGST
(DDM36),
and
GNSGSGKKKKKGKGSGLGSGKKKDPLGSGLGSGKKKKKGKGSGLG
SGKKKDPLGSTGGST
(DDM44), especially
GNSGSGKKKKKGKGSGLGSGKKKDPLGSGLGSGKKKKKGKGSGLG
SGKKKDPLGST
(DDM36),
and
GNSGSGKKKKKGKGSGLGSGKKKDPLGSGLGSGKKKKKGKGSGLG
SGKKKDPLGSTGGST
(DDM44).

4. Compound according to claim 1, wherein the compound additionally comprises a payload molecule to be delivered into a biological cell covalently attached to the polypeptide with the general formula (I), preferably a chemotherapeutic molecule, a cytotoxic molecule, a DNA damaging molecule, an anti-metabolite molecule, a therapeutic molecule, a small molecule with therapeutic effect inside a biological cell, a DNA molecule, an RNA molecule, an antibody molecule or an antibody derivative having an antibody-like function, a restriction endonuclease, a nicking enzyme, or DNA- or RNA-dependent endonucleases which show double strand breaking nuclease double strand/single strand breaking activity or no nuclease activity, more preferred wherein the payload molecule is a class II restriction endonuclease, such as PvuII, EcoRV, PvuII, HinfI, or a sc homodimer, a subunit or a functional fragment thereof, especially wherein the payload molecule is selected from the group
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSHPDLNKLLEL
WPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSHPDLNKLLEL
WPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAIYRLEPKDLEFYYDKW
ERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSHPDLNKLLEL
WPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCSGGSSHPDLNKLLE
LWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSHPDLNKLLEL
WPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAIYRLEPKDLEFYYDKW
ERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCSGGSSHPDLNKLLE
LWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAIYRLEPKDLEFYYDKW
ERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSHPDLNKLL
ELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIY,
and
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSHPDLNKLL
ELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAIYRLEPKDLEFYYDKW
ERKWYSDGHKDINNPKIPVKYVMEHGTKIY.

5. Compound according to claim 1, wherein the compound additionally comprises a homopolymer moiety covalently attached to the polypeptide with the general formula (I), preferably wherein the homopolymer is selected from the group of polyethylene glycol (PEG), especially a PEG with a MW of 5 to 30 kDa; dextran, polysialic acids, hyaluronic acid, dextrin, hydroxyethyl starch, or poly(2-ethyl 2-oxazoline.

6. Compound according to claim 1, wherein the compound additionally comprises a restriction endonuclease as a payload molecule to be delivered into a biological cell covalently attached to the polypeptide with the general formula (I), preferably a class II restriction endonuclease, especially a PvuII restriction endonuclease or a derivative thereof wherein the derivative is a single chain PvuII restriction endonuclease, such as the derivative comprising the amino acid sequence
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGG,
SGGSSHPDLNKLLELWPHIQEYQDLALKHGINDIFQDNGGKLLQVLLITGLTVLPGREGN
DAVDNAGQEYELKSINIDLTKGFSTHHHMNPVIIAKYRQVPWIFAIYRGIAIEAIYRLEPK
D-LEFYYDKWERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGNSG,
MSHPDLNKLLELWPHIQEYQDLALKHGINDIFQDNGGKLLQVLLITGLTVLPGREGNDA
VDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIY,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGC,
CSGGSSHPDLNKLLELWPHIQEYQDLALKHGINDIFQDNGGKLLQVLLITGLTVLPGREG
NDAVDNAGQEYELKSINIDLTKGFSTHHHMNPVIIAKYRQVPWIFAIYRGIAIEAIYRLEP
KD-LEFYYDKWERKWYSDGHKDINNPKIPVKYVMEHGTKIY,
MSHPDLNKLLELWPHIQEYQDLALKHGINDIFQDNGGKLLQVLLITGLTVLPGREGNDA
VDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSHPDLNKLLEL
WPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIY,
MSHPDLNKLLELWPHIQEYQDLALKHGINDIFQDNGGKLLQVLLITGLTVLPGREGNDA
VDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGSGGSSHPDLNKLLELW
PHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIY,
MSHPDLNKLLELWPHIQEYQDLALKHGINDIFQDNGGKLLQVLLITGLTVLPGREGNDA
VDNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGSGGS,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGSGGS,
or
GSGGSGGSSHPDLNKLLELWPHIQEYQDLALKHGINDIFQDNGGKLLQVLLITGLTVLPG
REGNDAVDNAGQEYELKSINIDLTKGFSTHHHMNPVIIA-
KYRQVPWIFAIYRGIAIEAIYRLEPKDLEFYYDKWERKWYSDGHKDINNPKIPVKYVME
HGTKIY.

7. Compound according to claim 1, wherein the polypeptide with the general formula (I) is covalently linked to another moiety by a linker, wherein the another moiety is preferably a payload molecule to be delivered into a biological cell, a labelling group, and/or a homopolymer.

8. Compound according to claim 1, wherein the compound further comprises a capping group, preferably an alkoxy, especially a methoxy, ethoxy, propoxy, or butoxy group; a halogen atom; or a tosylate; isocyanate, hydrazine hydrate, maleimide, orthopyridyl disulfide, N-succinimidyloxy, sulfo-N-succinimidyloxy, 1-benzotriazol, 1-imidazolyloxy, p-nitrophenyloxy, or aldehyde.

9. Compound according to claim 1, wherein the compound further comprises a linker, wherein the linker is an amino acid linker of 1 to 20 amino acid residues in length, preferably from 1 to 15 amino acid residues, more preferred from 7 to 13 amino acid residues, especially of 8 to 12 amino acid residues.

10. Compound according to claim 1, wherein the compound further comprises a linker, wherein the linker is an amino acid linker, selected from a linker comprising or consisting of cysteine, serine or glycine residues, preferably a single or two adjacent cysteine residue(s); an amino acid sequence comprising glycine and serine residues or comprising or consisting of the amino acid sequences GSG, SGG, GGC, GCS, CSG, GGS, GSGG, SGGS, GSGGC, CSGGS, GSGGSGGS, GSGGCCSGGS, GSGGCCCSGGS, or GSGGCSGGS.

11. Compound according to claim 1, wherein the compound further comprises as payload two monomeric subunits or parts thereof of a type II restriction endonuclease, preferably, two monomeric alpha-helical subunits of a type II restriction endonuclease covalently connected by a linker to the polypeptide with the general formula (I).

12. Compound according to claim 1, wherein the compound further comprises as payload two monomeric alpha-helical subunits of PvuII, preferably wherein the two monomeric subunits or parts thereof of the type II restriction endonuclease PvuII are covalently connected by a linker to the polypeptide with the general formula (I).

13. Compound according to claim 1, wherein the general formula (I) contains a single arginine and/or a single cysteine or not more than two arginine residues and/or not more than two cysteine residues

14. Compound according to claim 1, wherein the general formula (I) lacks arginine and/or cysteine, preferably wherein the general formula (I) lacks arginine and cysteine.

15. Compound according to claim 1, comprising at least two polypeptides with the general formula (I).

16. Compound according to claim 1, wherein the compound is selected from the group GNSGSGKKKKKGKGSGLGSGKKKDPL (DDM34) and
GNSGSGKKKKKGKGSGLGSGKKKDPLGSGLGSGKKKKKGKG
SGLGSGKKKDPLGST
(DDM36).

17. Compound according to claim 1, wherein the polypeptide with the general formula (I) is covalently coupled to the C-terminus of a payload molecule to be delivered into a biological cell, preferably a compound selected from the group
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGNSGSGKKKKKGKGSGLGS
GKKKDPLGSGLGSGKKKKKGKGSGLGSGKKKDPLGST,
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGNSGSGKKKKKGKGSGLGS
GKKKDPLGSGLGSGKKKKKGKGSGLGSGKKKDPLGST,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSHPDLNKLLEL
WPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGGSGGSSHPDLNKLLEL
WPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAIYRLEPKDLEFYYDKW
ERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSHPDLNKLLEL
WPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCSGGSSHPDLNKLLE
LWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCSGGSSHPDLNKLLEL
WPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAIYRLEPKDLEFYYDKW
ERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCSGGSSHPDLNKLLE
LWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAIYRLEPKDLEFYYDKW
ERKWYSDGHKDINNPKIPVKYVMEHGTKIYGNSGSGKKKKKGKGSGLG-
SGKKKDPLGSGLGSGKKKKKGKGSGLGSGKKKDPLGST,
SHPDLNKLLELWPHIQEYQDLALKHGINDIFQDNGGKLLQVLLITGLTVLPGREGNDAV
DNAGQEYELKSINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSHPDLNKLL
ELWPHIQEYQDLALKHGINDIFQDNGGKLLQVLLITGLTVLPGREGNDAVDNAGQE-
YELKSINIDLTKGFSTHHHMNPVIIAKYRQVPWIFAIYRGIAIEAIYRLEPKDLEFYYDKW
ERKWYSDGHKDINNPKIPVKYVMEHGTKIYGNSGSGKKKKKGKGSGLG-
SGKKKDPLGSGLGSGKKKKKGKGSGLGSGKKKDPLGST,
and
SHPDLNKLLELWPHIQEYQDLALKHGINDIFQDNGGKLLQVLLITGLTVLPGREGNDAV
DNAGQEYELASINIDLTKGFSTHHHMNPVIIAKYRQVPWIFAIYRGIAIEAIYRLEPKD-
LEFYYDKWERKWYSDGHKDINNPKIPVKYVMEHGTKIYGSGGCCCSGGSSHPDLNKLL
ELWPHIQEYQDLALKHGINDIFQDNGGKLLQVLLITGLTVLPGREGNDAVDNAGQE-
YELASINIDLTKGFSTHHHMNPVIIAKYRQVPWIFAIYRGIAIEAIYRLEPKDLEFYYDKW
ERKWYSDGHKDINNPKIPVKYVMEHGTKIYGNSGSGKKKKKGKGSGLG-
SGKKKDPLGSGLGSGKKKKKGKGSGLGSGKKKDPLGST.

18. Compound according to claim 1, wherein the polypeptide with the general formula (I) is covalently coupled to the C-terminus of a payload molecule to be delivered into a biological cell which is a class II restriction endonuclease, preferably PvuII or a subunit thereof, wherein the payload molecule is optionally attached at its C-terminus to a linker molecule comprising 8 to 12 amino acids comprising or consisting of glycine, serine and/or cysteine residues, and wherein the polypeptide with the general formula (I) is covalently coupled to the C-terminus of the payload molecule or the linker molecule, wherein the compound comprises one or two payload molecule(s), optionally separated by the linker molecule comprising 8 to 12 amino acids comprising or consisting of glycine, serine and/or cysteine residues.

19. Compound according to claim 1, wherein the polypeptide with the general formula (I) is covalently coupled to the C-terminus of a payload molecule to be delivered into a biological cell, wherein the compound comprises a first payload molecule which is the first subunit of PvuII linked at its C-terminus to a linker molecule comprising 8 to 12 amino acids comprising or consisting of glycine, serine and/or cysteine residues, wherein the linker molecule is attached at its C-terminus to the second subunit of PvuII and wherein the polypeptide with the general formula (I) is covalently coupled to the C-terminus of the second subunit of PvuII.

20. (canceled)

21. Method of treatment of a tumour patient wherein an effective amount of the compound according to claim 1 is administered to a patient in need thereof, preferably wherein the tumour patient is suffering from neuroblastoma, colon carcinoma, hepatocellular carcinoma, Small Cell Lung carcinoma, ovarian carcinoma, lung carcinoma, bladder carcinoma, prostate carcinoma, uterus carcinoma, cervix carcinoma, placental carcinoma, breast carcinoma, kidney carcinoma, liver carcinoma, pancreas carcinoma, muscle carcinoma, skin carcinoma, connective tissue carcinoma, bone carcinoma, and any of these carcinomas wherein the patient has already developed metastases, especially for the treatment of a patient having neuroblastoma, colon carcinoma, hepatocellular (liver) carcinoma, Small Cell Lung carcinoma, lung carcinoma, breast carcinoma, pancreas carcinoma, and any of these carcinomas wherein the patient has already developed metastases.

22. A method of manufacturing a medicament comprising the step of including a compound according to claim 1 in said medicament, preferably for the treatment of a tumour patient, more preferred for the treatment of a patient having neuroblastoma, colon carcinoma, hepatocellular carcinoma, Small Cell Lung carcinoma, ovarian carcinoma, lung carcinoma, bladder carcinoma, prostate carcinoma, uterus carcinoma, cervix carcinoma, placental carcinoma, breast carcinoma, kidney carcinoma, liver carcinoma, pancreas carcinoma, muscle carcinoma, skin carcinoma, connective tissue carcinoma, bone carcinoma, and any of these carcinomas wherein the patient has already developed metastases, especially for the treatment of a patient having neuroblastoma, colon carcinoma, hepatocellular (liver) carcinoma, Small Cell Lung carcinoma, lung carcinoma, breast carcinoma, pancreas carcinoma, and any of these carcinomas wherein the patient has already developed metastases.

23. Pharmaceutical preparation comprising a compound according to claim 1 and a protein kinase inhibitor (PKI), preferably a DNA-dependent PKI, more preferred a Non Homologous End Joining (NHEJ) and V(D)J repair factor DNA-dependent PKI, especially 7-Methyl-2-[(7-methyl[1,2,4]triazolo[1,5-a]pyridin-6-yl)amino]-9-(tetrahydro-2H-pyran-4-yl)-7,9-dihydro-8H-purin-8-one (AZD7648).


SEQUENCE LISTING (93 sequences)

SEQ ID NO: 1 - protein, 55 aa, synthetic construct
  REGION 1..19: note = peptide
  VARIANT 2: note = N or S
  VARIANT 3: note = S or T
  VARIANT 7..11: note = each X is independently R or K
  VARIANT 13: note = R or K
  VARIANT 14: note = S or L
  VARIANT 20..22: note = each X independently is R or K
  VARIANT 25: note = L or C
  VARIANT 33..37: note = each X independently is R or K
  VARIANT 39: note = R or K
  VARIANT 40: note = S or L
  VARIANT 46..48: note = each X independently is R or K
  VARIANT 51: note = L or C
  VARIANT 54: note = T or G
  VARIANT 55: note = G or L
GXXGSGXXXXXGXXGLGSGXXXDPXGSGLGSGXXXXXGXXGLGSGXXXDPXGSXX

SEQ ID NO: 2 - protein, 5 aa, synthetic construct
  REGION 1..5: note = peptide
KKKKK

SEQ ID NO: 3 - protein, 4 aa, synthetic construct
  REGION 1..4: note = peptide
KRKK

SEQ ID NO: 4 - protein, 4 aa, synthetic construct
  REGION 1..4: note = peptide
GLGS

SEQ ID NO: 5 - protein, 4 aa, synthetic construct
  REGION 1..4: note = peptide
GSGL

SEQ ID NO: 6 - protein, 6 aa, synthetic construct
  REGION 1..6: note = peptide
KYKPKL

SEQ ID NO: 7 - protein, 8 aa, synthetic construct
  REGION 1..8: note = peptide
KYKPKLGT

SEQ ID NO: 8 - skipped

SEQ ID NO: 9 - protein, 36 aa, synthetic construct
  REGION 1..36: note = peptide, DDM11
GNSGSGKRKKKGKGLGKKRDPCLRKYKPKLGTGSTG

SEQ ID NO: 10 - protein, 42 aa, synthetic construct
  REGION 1..42: note = peptide, DDM11
GNSGSGKRKKKGKGLGKKRDPCLRKYKPKLGTGSTGHHHHHH

SEQ ID NO: 11 - protein, 36 aa, synthetic construct
  REGION 1..36: note = peptide, DDM18
GNSGKRKKKGKGSGLGSGKKRDPCLRKYKPKLGSTG

SEQ ID NO: 12 - protein, 42 aa, synthetic construct
  REGION 1..42: note = peptide, DDM18
GNSGKRKKKGKGSGLGSGKKRDPCLRKYKPKLGSTGHHHHHH

SEQ ID NO: 13 - protein, 36 aa, synthetic construct; identical to SEQ ID NO: 9
  REGION 1..36: note = peptide, DDM21

SEQ ID NO: 14 - protein, 42 aa, synthetic construct; identical to SEQ ID NO: 10
  REGION 1..42: note = peptide, DDM21

SEQ ID NO: 15 - protein, 30 aa, synthetic construct
  REGION 1..30: note = peptide, DDM26
GNSGSGKRKKKGKGSGLGSGKKRDPLGSTG

SEQ ID NO: 16 - protein, 36 aa, synthetic construct
  REGION 1..36: note = peptide, DDM26
GNSGSGKRKKKGKGSGLGSGKKRDPLGSTGHHHHHH

SEQ ID NO: 17 - protein, 29 aa, synthetic construct
  REGION 1..29: note = peptide, DDM27
GNSGGSTGGKRKKKGKGSGLGSGKKRDPL

SEQ ID NO: 18 - protein, 35 aa, synthetic construct
  REGION 1..35: note = peptide, DDM27
GNSGHHHHHHGSTGGKRKKKGKGSGLGSGKKRDPL

SEQ ID NO: 19 - protein, 30 aa, synthetic construct
  REGION 1..30: note = peptide, DDM28
GNSGSGKRKKKGKGSGLGSGKKKDPLGSTG

SEQ ID NO: 20 - protein, 36 aa, synthetic construct
  REGION 1..36: note = peptide, DDM28
GNSGSGKRKKKGKGSGLGSGKKKDPLGSTGHHHHHH

SEQ ID NO: 21 - protein, 29 aa, synthetic construct
  REGION 1..29: note = peptide, DDM29
GNSGGSTGGKRKKKGKGSGLGSGKKKDPL

SEQ ID NO: 22 - protein, 35 aa, synthetic construct
  REGION 1..35: note = peptide, DDM29
GNSGHHHHHHGSTGGKRKKKGKGSGLGSGKKKDPL

SEQ ID NO: 23 - protein, 30 aa, synthetic construct
  REGION 1..30: note = peptide, DDM30
GNSGSGKKKKKGKGSGLGSGKKRDPLGSTG

SEQ ID NO: 24 - protein, 36 aa, synthetic construct
  REGION 1..36: note = peptide, DDM30
GNSGSGKKKKKGKGSGLGSGKKRDPLGSTGHHHHHH

SEQ ID NO: 25 - protein, 29 aa, synthetic construct
  REGION 1..29: note = peptide, DDM31
GNSGGSTGGKKKKKGKGSGLGSGKKRDPL

SEQ ID NO: 26 - protein, 35 aa, synthetic construct
  REGION 1..35: note = peptide, DDM31
GNSGHHHHHHGSTGGKKKKKGKGSGLGSGKKRDPL

SEQ ID NO: 27 - protein, 30 aa, synthetic construct
  REGION 1..30: note = peptide, DDM32
GNSGSGKKKKKGKGSGLGSGKKKDPLGSTG

SEQ ID NO: 28 - protein, 36 aa, synthetic construct
  REGION 1..36: note = peptide, DDM32
GNSGSGKKKKKGKGSGLGSGKKKDPLGSTGHHHHHH

SEQ ID NO: 29 - protein, 29 aa, synthetic construct
  REGION 1..29: note = peptide, DDM33
GNSGGSTGGKKKKKGKGSGLGSGKKKDPL

SEQ ID NO: 30 - protein, 35 aa, synthetic construct
  REGION 1..35: note = peptide, DDM33
GNSGHHHHHHGSTGGKKKKKGKGSGLGSGKKKDPL

SEQ ID NO: 31 - protein, 28 aa, synthetic construct
  REGION 1..28: note = peptide, DDM35
GNSGSGKKKKKGKGSGLGSGKKKLGSTG

SEQ ID NO: 32 - protein, 34 aa, synthetic construct
  REGION 1..34: note = peptide, DDM35
GNSGSGKKKKKGKGSGLGSGKKKLGSTGHHHHHH

SEQ ID NO: 33 - protein, 60 aa, synthetic construct
  REGION 1..60: note = peptide, DDM44
GNSGSGKKKKKGKGSGLGSGKKKDPLGSGLGSGKKKKKGKGSGLGSGKKKDPLGSTGGST

SEQ ID NO: 34 - protein, 26 aa, synthetic construct
  REGION 1..26: note = peptide, DDM34
GNSGSGKKKKKGKGSGLGSGKKKDPL

SEQ ID NO: 35 - protein, 66 aa, synthetic construct
  REGION 1..66: note = peptide, DDM44
GNSGSGKKKKKGKGSGLGSGKKKDPLGSGLGSGKKKKKGKGSGLGSGKKKDPLGSTGHHHHHHGST

SEQ ID NO: 36 - protein, 56 aa, synthetic construct
  REGION 1..56: note = peptide, DDM36
GNSGSGKKKKKGKGSGLGSGKKKDPLGSGLGSGKKKKKGKGSGLGSGKKKDPLGST

SEQ ID NO: 37 - protein, 156 aa, synthetic construct
  REGION 1..156: note = peptide
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 38 - protein, 160 aa, synthetic construct
  REGION 1..160: note = peptide
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGG

SEQ ID NO: 39 - protein, 160 aa, synthetic construct
  REGION 1..160: note = peptide
SGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 40 - protein, 160 aa, synthetic construct
  REGION 1..160: note = peptide
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGNSG

SEQ ID NO: 41 - protein, 157 aa, synthetic construct
  REGION 1..157: note = peptide
MSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 42 - protein, 161 aa, synthetic construct
  REGION 1..161: note = peptide
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGC

SEQ ID NO: 43 - protein, 161 aa, synthetic construct
  REGION 1..161: note = peptide
CSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 44 - protein, 322 aa, synthetic construct
  REGION 1..322: note = peptide
MSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGCSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 45 - protein, 321 aa, synthetic construct
  REGION 1..321: note = peptide
MSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 46 - protein, 165 aa, synthetic construct
  REGION 1..165: note = peptide
MSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGSGGS

SEQ ID NO: 47 - protein, 164 aa, synthetic construct
  REGION 1..164: note = peptide
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGSGGS

SEQ ID NO: 48 - protein, 164 aa, synthetic construct
  REGION 1..164: note = peptide
GSGGSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 49 - protein, 377 aa, synthetic construct
  REGION 1..377: note = peptide, CROMOC36
MSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 50 - protein, 156 aa, synthetic construct; identical to SEQ ID NO: 37
  REGION 1..156: note = peptide

SEQ ID NO: 51 - protein, 160 aa, synthetic construct; identical to SEQ ID NO: 38
  REGION 1..160: note = peptide

SEQ ID NO: 52 - protein, 160 aa, synthetic construct; identical to SEQ ID NO: 39
  REGION 1..160: note = peptide

SEQ ID NO: 53 - protein, 160 aa, synthetic construct; identical to SEQ ID NO: 40
  REGION 1..160: note = peptide

SEQ ID NO: 54 - protein, 157 aa, synthetic construct; identical to SEQ ID NO: 41
  REGION 1..157: note = peptide

SEQ ID NO: 55 - protein, 161 aa, synthetic construct; identical to SEQ ID NO: 42
  REGION 1..161: note = peptide

SEQ ID NO: 56 - protein, 161 aa, synthetic construct; identical to SEQ ID NO: 43
  REGION 1..161: note = peptide

SEQ ID NO: 57 - protein, 322 aa, synthetic construct; identical to SEQ ID NO: 44
  REGION 1..322: note = peptide

SEQ ID NO: 58 - protein, 321 aa, synthetic construct; identical to SEQ ID NO: 45
  REGION 1..321: note = peptide

SEQ ID NO: 59 - protein, 165 aa, synthetic construct; identical to SEQ ID NO: 46
  REGION 1..165: note = peptide

SEQ ID NO: 60 - protein, 377 aa, synthetic construct
  REGION 1..377: note = peptide, CROMOC60
MSHPDLNKLLELWPHIQEYQDLALKHGINDIFQDNGGKLLQVLLITGLTVLPGREGNDAVDNAGQEYELKSINIDLTAGFSTHHHMNPVIIAKYRQVPWIFAIYRGIAIEAIYRLEPKDLEFYYDKWERKWYSDGHKDINNPKIPVKYVMEHGTKIYGSGGSGGSSHPDLNKLLELWPHIQEYQDLALKHGINDIFQDNGGKLLQVLLITGLTVLPGREGNDAVDNAGQEYELKSINIDLTAGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 61 - protein, 164 aa, synthetic construct; identical to SEQ ID NO: 47
  REGION 1..164: note = peptide

SEQ ID NO: 62 - protein, 378 aa, synthetic construct
  REGION 1..378: note = peptide, CROMOC62
MSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGCSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 63 - protein, 164 aa, synthetic construct; identical to SEQ ID NO: 48
  REGION 1..164: note = peptide

SEQ ID NO: 64 - protein, 4 aa, synthetic construct
  REGION 1..4: note = peptide
GSGG

SEQ ID NO: 65 - protein, 4 aa, synthetic construct
  REGION 1..4: note = peptide
SGGS

SEQ ID NO: 66 - protein, 5 aa, synthetic construct
  REGION 1..5: note = peptide
GSGGC

SEQ ID NO: 67 - protein, 5 aa, synthetic construct
  REGION 1..5: note = peptide
CSGGS

SEQ ID NO: 68 - protein, 8 aa, synthetic construct
  REGION 1..8: note = peptide
GSGGSGGS

SEQ ID NO: 69 - protein, 9 aa, synthetic construct
  REGION 1..9: note = peptide
GSGGCSGGS

SEQ ID NO: 70 - protein, 15 aa, synthetic construct
GKRKKKGKLGKKRDP

SEQ ID NO: 71 - protein, 22 aa, synthetic construct
GKRKKKGKGLGKKRDPCLRKYK

SEQ ID NO: 72 - protein, 10 aa, synthetic construct
GSGGCCSGGS

SEQ ID NO: 73 - protein, 156 aa, synthetic construct; identical to SEQ ID NO: 37

SEQ ID NO: 74 - protein, 156 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 75 - protein, 321 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGGSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 76 - protein, 321 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGSGGGSGGSSHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 77 - protein, 321 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGCSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 78 - protein, 322 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGCCSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 79 - protein, 321 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGSGGCSGGSSHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 80 - protein, 322 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGSGGCCSGGSSHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 81 - protein, 323 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGCCCSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 82 - protein, 323 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGSGGCCCSGGSSHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIY

SEQ ID NO: 83 - protein, 212 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 84 - protein, 212 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 85 - protein, 377 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGGSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 86 - protein, 377 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGSGGGSGGSSHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 87 - protein, 377 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGCSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 88 - protein, 378 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGCCSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 89 - protein, 377 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGSGGCSGGSSHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 90 - protein, 378 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGSGGCCSGGSSHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 91 - protein, 379 aa, synthetic construct
SHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGSGGCCCSGGSSHPDLNKLLELWPHIQEYQDLALKHGINDIFQDNGGKLLQVLLITGLTVLPGREGNDAVDNAGQEYELKSINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 92 - protein, 379 aa, synthetic construct
SHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGSGGCCCSGGSSHPDLNKLLELWPHIQEYQDLALKHGINDIFQDNGGKLLQVLLITGLTVLPGREGNDAVDNAGQEYELASINIDLTKGFSTHHHMNPVIIAKYRQVPWIFAIYRGIAIEAIYRLEPKDLEFYYDKWERKWYSDGHKDINNPKIPVKYVMEHGTKIYGNSGSGKKKKKGKGSGLGSGKKKDPLGSGLGSGKKKKKGKGSGLGSGKKKDPLGST

SEQ ID NO: 93 - protein, 11 aa, synthetic construct
GSGGCCCSGGS